(19) United States
(12) Patent Application Publication
(10) Pub. No.: US 2025/0255850 A1
(43) Pub. Date: Aug. 14, 2025

(54) PHARMACEUTICAL COMPOSITIONS FOR TREATING NEUROLOGICAL CONDITIONS

(71) Applicant: YISSUM RESEARCH DEVELOPMENT COMPANY OF THE HEBREW UNIVERSITY OF JERUSALEM LTD., Jerusalem (IL)

(72) Inventors: Haitham Amal, Haifa (IL); Abraham Jacob Domb, Jerusalem (IL)

(21) Appl. No.: 19/173,097

(22) Filed: Apr. 8, 2025

Related U.S. Application Data
(63) Continuation of application No. 18/290,220, filed on Nov. 10, 2023.
(60) Provisional application No. 63/186,389, filed on May 10, 2021.

(51) Int. Cl. A61K 31/416; A61K 47/10; A61K 47/14; A61K 47/26; A61K 47/44; A61P 25/28; A61P 35/00

(52) U.S. Cl. CPC A61K 31/416; A61K 47/10; A61K 47/14; A61K 47/26; A61K 47/44; A61P 25/28; A61P 35/00

(57) ABSTRACT

Method and compositions that comprise an agent that reduces nNOS activity and uses thereof in the treatment of a disease or condition in which a beneficial clinical effect is achieved by reduction in neuronal nitric oxide synthase (nNOS) activity are provided.

### SEQUENCE LISTING STATEMENT

The XML file, entitled 2025 Apr. 8-Sequence Listing 23573-008US2-AMAL1B, created on Apr. 7, 2025, comprising 17,009 bytes in size, submitted concurrently with the filing of this application is incorporated herein by reference in its entirety.

### FIELD AND BACKGROUND OF THE INVENTION

The present invention, in some embodiments thereof, relates to therapy, and, more particularly, but not explicitly, to pharmaceutical compositions and methods for treating neurological conditions associated with abnormal activity of neuronal nitric oxide synthase (nNOS) such as, but not limited to, autistic spectrum disorders (ASDs) and neural-derived cancer . . .

Nitric oxide (herein after “NO”), is the smallest signaling molecule. NO is also referred to as a “double-edged sword”. A large body of accumulated evidence suggests that NO is one of the key factors in the genesis of many brain-related disorders. S-nitrosylation (SNO), the NO-mediated post-translational modification on the cysteine residue, targets a wide range of prominent intracellular proteins leading to alteration in signaling pathways, which may converge into synaptic, neuronal and behavioral deficits. Thus, although NO promotes neurogenesis, aberrant SNO may be responsible for different neurodevelopmental disorders, such as autism spectrum disorders (ASD). This “double-edge sword” function is also dependent on the concentration of NO. At low concentrations, NO serves as a signaling molecule, which takes part in the regulation of synaptic activity, synaptic plasticity, and vesicle trafficking. However, at high concentrations, NO might be toxic, possibly leading to modified phenotypes and cell death. It reacts with superoxide radical (O₂⁻) and forms peroxynitrite (ONOO⁻) which ultimately induces damage to DNA, lipids, and proteins during oxidative stress.

NO may affect cellular signaling through proteins S-nitrosylation (SNO), tyrosine nitration, and S-nitrosogluthanione (GSNO) formation. Protein tyrosine nitration under stress conditions (NO overproduction) has been considered as a potential marker for nitrosative stress. 3-nitrotyrosine (Ntyr) is a product of tyrosine nitration mediated by peroxynitrite, which acts as nitrating agent.

SNO is a reversible NO-mediated post-translational modification of cysteine thiols in which cysteine is converted to nitrosothiol. SNO plays a major role in the localization and activity of a wide range of key enzymes and receptors leading to modulation of many signaling pathways, axonal transport, synaptic plasticity, and protein assembly. Thus, aberrant SNO signaling and increase in Ntyr may contribute to the progression of many neurodegenerative, neurodevelopmental, and neuropsychiatric disorders as well as neural tumors, as summarized below.

Since NO cannot be stored in the cells, new synthesis is necessary to mediate its activities.

NO is produced by the three types of NO synthases, nNOS, iNOS and eNOS. nNOS, which stands for neuronal nitric oxide synthase, is found in neuronal tissues.

Nitric oxide synthases (EC 1.14.13.39) (NOSs) are a family of enzymes catalyzing the production of nitric oxide (NO) from L-arginine. In mammals, there are three distinct genes encode NOS isozymes: neuronal (nNOS or NOS-1), cytokine-inducible (iNOS or NOS-2) and endothelial (eNOS or NOS-3). iNOS and nNOS are soluble and found predominantly in the cytosol, while eNOS is membrane associated.

Neuronal nitric oxide synthase (nNOS) produces nitric oxide (NO) in nervous tissue in both the central and peripheral nervous systems. Its functions include: Synaptic plasticity in the central nervous system (CNS); Synaptic transmission and axon elongation in the peripheral nervous systems and CNS; and Aberrant signaling that leads to multiple neurological disorders nNOS also performs a role in cell communication and is associated with plasma membranes.

The subcellular localization of nNOS in skeletal muscle is mediated by anchoring of nNOS to dystrophin. nNOS contains an additional N-terminal domain, the PDZ domain. The gene coding for nNOS is located on Chromosome 12.

nNOS has been found in neurons, astrocytes, the adventitia of brain blood vessels and cardiac myocytes. Besides brain tissue, nNOS has been found by immunohistochemistry in other tissues. nNOS is known to be specifically inhibited by 7-nitroindazole, which acts as a non-competitive substrate inhibitor.

It has been shown that the pathogenesis of ASD, at least in part, can be associated with synaptic dysfunction, which can lead to functional and cognitive impairments (1). Previous studies have shown that mutations in genes, such as SHANK3 and CNTNAP2 and others converge on common synapse-related cellular pathways, which are strongly related to ASD (2).

It has been found that NO-mediated post-translational modification (PTM) of cysteine thiols (SNO) in the mutant model SHANK3 human gene leads to different neuropsychiatric diseases including Autism Spectrum Disorder (ASD). Pathway analysis showed enrichment of processes affected in ASD. A significant increase of 3-nitrotyrosine was found in the cortical regions of the adult mutant, signaling both oxidative and nitrosative stress (5). Interestingly, nNOS knockdown (6) exhibited an ASD-like phenotype in cultured cells.

Neuroblastoma (NB) is referred to a spectrum of neuroblastic tumors that originate from the neural crest cells during fetal development. Pediatric cancers, like NB, account for 97% of all neuroblastic tumors. NB is the most common tumor among children less than one year of age worldwide.

Despite extensive efforts, the underlying mechanisms of NB remain largely obscure. One of the essential regulators of carcinogenesis of different tumors including NB is nitric oxide (NO). It will be appreciated that the process of SNO, as described above, can be associated with the development of different cancers (Mishra et al., 2020). This post-translational modification (PTM) has been reported to confer either tumor-suppressing or tumor-promoting effects and is described as a process involved in every stage of cancer progression (Sharma et al., 2021). One of the pathways that can be affected by SNO is the mechanistic target of rapamycin (mTOR), an important regulator of cell proliferation, metabolism, and tumorigenesis (Johnsen et al., 2008). In a previous study, using the SNO-proteome analysis of the mouse brain, it was found that the mTOR pathway is significantly amplified by SNO when the NO levels were elevated (Mencer et al., 2021).

Additional background art includes:
Haim-Zada et al. 2017. Stable polyanhydride synthesized from sebacic acid and ricinoleic acid. J. Controlled Release, 257:156-162;
Aquilano K, Filomeni G, Baldelli S, Piccirillo S, De Martino A, Rotilio G, Ciriolo M R (2007) Neuronal nitric oxide synthase protects neuroblastoma cells from oxidative stress mediated by garlic derivatives. J Neurochem 101:1327-1337;
Berry T et al. (2012) The ALKF1174L mutation potentiates the oncogenic activity of MYCN in neuroblastoma. Cancer Cell 22:117-130;
Burke A J, Sullivan F J, Giles F J, Glynn S A (2013) The yin and yang of nitric oxide in cancer progression. Carcinogenesis 34:503-512;
Ciani E, Guidi S, Della Valle G, Perini G, Bartesaghi R, Contestabile A (2002) Nitric oxide protects neuroblastoma cells from apoptosis induced by serum deprivation through cAMP-response element-binding protein (CREB) activation. J Biol Chem 277:49896-49902;
Corasaniti M, Melino G, Navarra M, Garaci E, Finazzi-Agro A, Nistico G (1995) Death of cultured human neuroblastoma cells induced by HIV-1 gp120 is prevented by NMDA receptor antagonists and inhibitors of nitric oxide and cyclooxygenase. Neurodegeneration 4:315-321;
Fujibayashi T, Kurauchi Y, Hisatsune A, Seki T, Shudo K, Katsuki H J Jops (2015) Mitogen-activated protein kinases regulate expression of neuronal nitric oxide synthase and neurite outgrowth via non-classical retinoic acid receptor signaling in human neuroblastoma SH-SY5Y cells. 129:119-126;
Gao R-N, Levy I G, Woods W G, Coombs B A, Gaudette L A, Hill G (1997) Incidence and mortality of neuroblastoma in Canada compared with other childhood cancers. Cancer Causes Control 8:745-754;
Gordon J L, Hinsen K J, Reynolds M M, Smith T A, Tucker H O, Brown M A (2021) Anticancer potential of nitric oxide (NO) in neuroblastoma treatment. RSC Adv 11:9112-9120;
Hickok J R, Thomas D D (2010) Nitric oxide and cancer therapy: the emperor has NO clothes. Curr Pharm Des 16:381-391;
Huang Z, Fu J, Zhang Y (2017) Nitric oxide donor-based cancer therapy: advances and prospects. J Med Chem 60:7617-7635;
Kiessling M K, Curioni-Fontecedro A, Samaras P, Lang S, Scharl M, Aguzzi A, Oldrige D A, Maris J M, Rogler G (2016) Targeting the mTOR complex by everolimus in NRAS mutant neuroblastoma. PLoS One 11: e0147682;
Lamant L, Pulford K, Bischof D, Morris S W, Mason D Y, Delsol G, Mariamé B (2000) Expression of the ALK tyrosine kinase gene in neuroblastoma. Am J Pathol 156:1711-1721;
Lange I, Koster J, Koomoa D-L T (2019) Calcium signaling regulates fundamental processes involved in Neuroblastoma progression. Cell Calcium 82:102052;
Lopez-Rivera E, Jayaraman P, Parikh F, Davies M A, Ekmekcioglu S, Izadmehr S, Milton D R, Chipuk J E, Grimm E A, Estrada YJCr (2014) Inducible nitric oxide synthase drives mTOR pathway activation and proliferation of human melanoma by reversible nitrosylation of TSC2. 74:1067-1078;
Ortiz-Ortiz M A, Morán J M, González-Polo R A, Niso-Santano M, Soler G, Bravo-San Pedro J M, Fuentes JMJNr (2009) Nitric oxide-mediated toxicity in paraquat-exposed SH-SY5Y cells: a protective role of 7-nitroindazole. 16:160-173;
Tripathi M K, Kartawy M, Amal H (2020) The role of nitric oxide in brain disorders: Autism spectrum disorder and other psychiatric, neurological, and neurodegenerative disorders. Redox Biol: 101567; and
Vahora H, Khan M A, Alalami U, Hussain A (2016) The potential role of nitric oxide in halting cancer progression through chemoprevention. J Cancer Prev 21:1.

### SUMMARY OF THE INVENTION

According to an aspect of some embodiments of the present invention there is provided a method of treating a disease or condition in which a beneficial clinical effect is achieved by reduction in neuronal nitric oxide synthase (nNOS) activity, the method comprising administering to a subject in need thereof an effective amount of a composition comprising an active agent which reduces nNOS activity, as describe herein in any of the respective embodiments, thereby treating 5 the disease or condition in which a beneficial clinical effect is achieved by reduction in nNOS activity.

According to an aspect of some embodiments of the present invention there is provided a pharmaceutical composition comprising an active agent which reduces nNOS activity, as describe herein in any of the respective embodiments, and optionally a pharmaceutically acceptable carrier, for use treating the disease or condition in which a beneficial clinical effect is achieved by reduction in nNOS activity.

According to some of any of the embodiments described herein, the reduction in nNOS activity is in the central nervous system (CNS).

According to some of any of the embodiments described herein, the reduction in nNOS activity is selectively or preferentially in the CNS of the subject as compared to other tissues.

According to some of any of the embodiments described herein, the reduction is a reduction in nNOS activity in the brain.

According to some of any of the embodiments described herein, the composition comprises an active agent which is nNOS specific.

According to some of any of the embodiments described herein, the administering mode achieves a reduction of nNOS activity selectively and preferentially in the CNS.

According to some of any of the embodiments described herein, the administering mode comprises local administration.

According to some of any of the embodiments described herein, the composition comprises a carrier which carries the agent to the CNS of the subject.

According to some of any of the embodiments described herein, the carrier comprises an adeno-associated virus.

According to some of any of the embodiments described herein, the agent decreases nNOS expression.

According to some of any of the embodiments described herein, the agent decreases nNOS translation.

According to some of any of the embodiments described herein, the agent inhibits nNOS enzymatic activity.

According to some of any of the embodiments described herein, the agent is selected from the group consisting of a small molecule, a competing peptide and an antibody or a fragment thereof.

According to some of any of the embodiments described herein, the agent binds to an active site of nNOS and blocks substrate binding.

According to some of any of the embodiments described herein, the agent is N^{ω}-Nitroarginine, or any small molecule agent as described herein.

According to some of any of the embodiments described herein, the agent is represented by the Formula I:

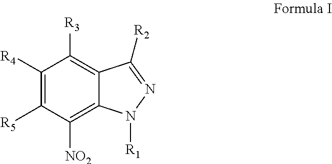

or a pharmaceutically acceptable salt thereof,
wherein:
R₁ is selected from hydrogen, alkyl and cycloalkyl and is preferably hydrogen; and
R₂-R₅ are each independently selected from hydrogen, alkyl, cycloalkyl, aryl, heteroalicyclic, heteroaryl, halo, haloalkyl, hydroxy, alkoxy, aryloxy, thiol, amine, carboxylate, thiocarboxylate, and a poly(alkylene glycol) moiety.

According to some of any of the embodiments described herein, R₁ is hydrogen.

According to some of any of the embodiments described herein, each of R₂-R₅ is hydrogen.

According to some of any of the embodiments described herein, the compound of Formula I is in a form of an anionic salt thereof.

According to some of any of the embodiments described herein, the compound of Formula I is in a form of a cationic salt thereof.

According to some of any of the embodiments described herein, the agent is an amino acid based inhibitor.

According to some of any of the embodiments described herein, the agent is PIN or nitric oxide synthase interacting protein (NOSIP).

According to some of any of the embodiments described herein, the agent is a selective nNOS inhibitor.

According to some of any of the embodiments described herein, the agent reduces a level of an NO precursor.

According to some of any of the embodiments described herein, the agent increases a level of GSNO reductase level.

According to some of any of the embodiments described herein, the agent is a nNOS-specific nucleic acid sequence.

According to some of any of the embodiments described herein, the agent is an antisense or a siRNA.

According to some of any of the embodiments described herein, the agent is a DNA editing agent.

According to some of any of the embodiments described herein, the agent is CRISPR/Cas9 for selectively reducing expression of nNOS.

According to some of any of the embodiments described herein, a carrier for the agent is a viral delivery vector.

According to some of any of the embodiments described herein, the disease or condition is a neurological disease or condition.

According to some of any of the embodiments described herein, the disease or condition is a brain disorder.

According to some of any of the embodiments described herein, the disease or condition is an autism spectrum disorder (ASD).

According to some of any of the embodiments described herein, the disease or condition is ADD or ADHD.

According to some of any of the embodiments described herein, the disease or condition is selected from the group consisting of Alzheimer's disease, Parkinson's disease, Huntington's disease, multiple sclerosis, schizophrenia, addiction, amyotrophic lateral sclerosis (ALS), epilepsy, bipolar disease and migraine.

According to some of any of the embodiments described herein, the disease or condition is a neurodevelopmental disorder, a psychiatric disorder or a neurodegenerative disease.

According to some of any of the embodiments described herein, the disease or condition is a cancer of a neural origin or a neuroectodermal origin.

According to some of any of the embodiments described herein, the cancer is selected from the group consisting of neuroblastoma, glioma, ganglioglioma, central neurocytoma, ganglioneuroblastoma, medulloblastoma and a primitive neuroectodermal tumor (PNET).

According to some of any of the embodiments described herein, the neuroblastoma comprise a mutation in ALK.

According to some of any of the embodiments described herein, the administering is to a striatum and/or cortex of the CNS of the subject.

According to some of any of the embodiments described herein, the composition comprises a CNS (e.g., brain)-selective delivery vehicle.

According to some of any of the embodiments described herein, the brain-selective delivery vehicle comprises nanoparticles, liposomes or exosomes.

According to some of any of the embodiments described herein, the delivery vehicle comprises a ligand or receptor to enhance uptake across the BBB of the subject.

According to some of any of the embodiments described herein, the composition is administered along with ultrasound or magnetic stimulation to enhance entry through the BBB of the subject.

According to some of any of the embodiments described herein, upon administration of the composition, a level of physiologically available nitric oxide in neuronal cells of the subject is reduced by at least 20%.

According to some of any of the embodiments described herein, upon administration of the composition, a concentration of nitro-tyrosine in the plasma of the subject is reduced by at least 20%.

According to an aspect of some embodiments of the present invention there is provided a pharmaceutical composition comprising a therapeutically active agent which reduces neuronal nitric oxide synthase (nNOS) activity, as described herein in any of the respective embodiments, and a pharmaceutically acceptable carrier, wherein the composition and the therapeutically active agent are selected such that upon administration of the composition to a subject, reduction in the nNOS activity is effected selectively or preferentially in the central nervous system (CNS) or in the brain of the subject as compared to other tissues.

According to some of any of the embodiments described herein, reduction in nNOS activity in neuronal cells (e.g., expressing nNOS) or in the CNS (e.g., the brain) of the subject is by at least 20%, or by at least 30%, or by at least 40%. According to some embodiments, a reduction in NOS activity is other tissues or organs is no more than 10%, or no more than 5%, or no more than 2%, or no more than 1%.

According to some of any of the embodiments described herein, reduction in nNOS activity in neuronal cells (e.g., expressing nNOS) or in the CNS (e.g., the brain) of the subject is higher by at least 20%, or by at least 30%, or by at least 40% compared to other tissues or organs.

According to some of any of the embodiments described herein, the composition is configured for local administration to the CNS of the subject.

According to some of any of the embodiments described herein, the composition is configured for local administration to the brain of the subject.

According to some of any of the embodiments described herein, the composition is configured to deliver the therapeutically active agent selectively to the central nervous system of the subject.

According to some of any of the embodiments described herein, the composition is configured to deliver the therapeutically active agent selectively to the brain of the subject.

According to some of any of the embodiments described herein, the therapeutically active agent is selective to nNOS.

According to some of any of the embodiments described herein, the composition is configured to deliver the therapeutically active agent selectively to the brain or the central nervous system for an extended time period.

According to some of any of the embodiments described herein, the composition is configured to deliver an amount of the therapeutically active agent sufficient to reduce nNOS activity in the brain during a time period of at least 1 day, or at least one week, at least 12 days.

According to some of any of the embodiments described herein, the pharmaceutical composition is formulated for oral, nasal or buccal delivery or administration.

According to some of any of the embodiments described herein, the carrier comprises an aqueous solution.

According to some of any of the embodiments described herein, the carrier comprises a mixture of at least one lipid, at least one of a surfactant, and a water-miscible organic solvent, dispersed in an aqueous solution.

According to some of any of the embodiments described herein, the pharmaceutical composition is in a form of a tablet, a capsule, a syrup, a solution, a spray, an aerosol or a dispersion.

According to some of any of the embodiments described herein, pharmaceutical composition is formulated for administration by injection.

According to some of any of the embodiments described herein, the carrier comprises an aqueous solution.

According to some of any of the embodiments described herein, the carrier comprises a mixture of at least one lipid, at least one of a surfactant, and a water-miscible organic solvent, and according tom some embodiments the mixture forms lipid nanoparticles dispersed in an aqueous solution upon contacting an aqueous solution.

According to some of any of the embodiments described herein, the carrier forms a depot for an extended release of the therapeutically active agent.

According to some of any of the embodiments described herein, the carrier is a polymeric carrier, preferably comprising at least one biodegradable polymer.

According to some of any of the embodiments described herein, the polymeric carrier comprises poly(sebacic-co-ricinoleic) acid.

According to some of any of the embodiments described herein, the polymeric carrier comprises PLGA, PLA, PCL, polycarbonate or a combination thereof.

According to some of any of the embodiments described herein, the carrier is an aqueous carrier and the therapeutically active agent is dissolvable in the aqueous carrier.

According an aspect of some embodiments of the present invention, there is provided pharmaceutical composition as described herein in any of the respective embodiments, for use in treating a disease or condition in which a beneficial clinical effect is achieved by reduction in nNOS activity, as described herein.

According to an aspect of some embodiments of the present invention there is provided a pharmaceutical composition comprising an agent that reduces nNOS activity and which is nNOS specific and a pharmaceutically acceptable carrier, the composition being formulated for oral delivery or administration.

According to an aspect of some embodiments of the present invention there is provided a pharmaceutical composition comprising a compound of Formula I or a pharmaceutically acceptable salt thereof as described herein in any of the respective embodiments, and a pharmaceutically acceptable carrier, the composition being formulated for oral, nasal or buccal delivery or administration.

According to some of any of the embodiments described herein, the carrier comprises an aqueous solution.

According to some of any of the embodiments described herein, the compound comprises a pharmaceutically acceptable salt thereof which is water-dissolvable or water-immiscible.

According to some of any of the embodiments described herein, the carrier further comprises lipid nanoparticles dispersed in the aqueous solution.

According to some of any of the embodiments described herein, the pharmaceutical composition is for oral administration to a subject in need thereof between one and four times per day.

According to an aspect of some embodiments of the present invention there is provided a pharmaceutical composition comprising an agent that reduces nNOS activity and which is nNOS specific and a pharmaceutically acceptable carrier, wherein the carrier comprises an aqueous solution and lipid nanoparticles dispersed in the aqueous solution.

According to an aspect of some embodiments of the present invention there is provided a pharmaceutical composition comprising a compound of Formula I or a pharmaceutically acceptable salt thereof as described herein in any of the respective embodiments and a pharmaceutically acceptable carrier, wherein the carrier comprises an aqueous solution and lipid nanoparticles dispersed in the aqueous solution.

According to some of these embodiments, the carrier is formed upon contacting a PNL formulation as described herein with an aqueous solution.

According to an aspect of some embodiments of the present invention there is provided a pharmaceutical composition comprising an agent that reduces nNOS activity and which is nNOS specific and a pharmaceutically acceptable carrier, wherein the carrier comprises a mixture of at least one lipid, at least one surfactant (preferably hydrophilic), and a water-miscible organic solvent, and forms, upon contacting an aqueous solution or medium (e.g., physiological medium), a dispersion of lipid nanoparticles in the aqueous solution or medium.

According to an aspect of some embodiments of the present invention there is provided a pharmaceutical composition comprising a compound of Formula I or a pharmaceutically acceptable salt thereof as described herein in any of the respective embodiments and a pharmaceutically acceptable carrier, wherein the carrier comprises a mixture of at least one lipid, at least one surfactant (preferably hydrophilic), and a water-miscible organic solvent, and forms, upon contacting an aqueous solution or medium (e.g., physiological medium), a dispersion of lipid nanoparticles in the aqueous solution or medium.

According to an aspect of some embodiments of the present invention there is provided a pharmaceutical composition comprising an agent that reduces nNOS activity as described herein in any of the respective embodiments and a pharmaceutically acceptable carrier, the composition being formulated for releasing the composition of Formula I or the salt thereof during a time period of at least one day, or of at least one week.

According to an aspect of some embodiments of the present invention there is provided a pharmaceutical composition comprising a compound of Formula I or a pharmaceutically acceptable salt thereof as described herein in any of the respective embodiments and a pharmaceutically acceptable carrier, the composition being formulated for releasing the composition of Formula I or the salt thereof during a time period of at least one day, or of at least one week.

According to some of any of the embodiments described herein, pharmaceutical composition is formulated for administration by injection.

According to some of any of the embodiments described herein, the carrier is a polymeric carrier, preferably comprising at least one biodegradable polymer.

According to some of any of the embodiments described herein, the polymeric carrier comprises poly(sebacic-co-ricinoleic) acid.

According to some of any of the embodiments described herein, the polymeric carrier comprises PLGA, PLA, PCL, polycarbonate or a combination thereof.

According to an aspect of some embodiments of the present invention there is provided a pharmaceutical composition as described herein in any of the respective embodiments, for use in treating a medical condition in which a beneficial clinical effect is achieved by reduction in neuronal nitric oxide synthase (nNOS) activity.

According to an aspect of some embodiments of the present invention there is provided a pharmaceutical composition as described herein in any of the respective embodiments, for use in treating a medical condition in which a beneficial effect is achieved by reducing a level of physiologically available nitric oxide in neuronal cells of a subject by at least 20%.

According to an aspect of some embodiments of the present invention there is provided a method of treating an autism spectrum disorder in a subject in need thereof, the method comprising administering to the subject an effective amount of a composition comprising an active agent which reduces nNOS activity, as described herein in any of the respective embodiments, wherein reduction in the nNOS activity is selectively or preferentially in the brain of the subject as compared to other tissues, thereby treating the autism spectrum disorder.

According to an aspect of some embodiments of the present invention there is provided a method of treating an autism spectrum disorder in a subject in need thereof, the method comprising administering to the subject an effective amount of a pharmaceutical composition as described herein in any of the respective embodiments, thereby treating the autism spectrum disorder.

According to an aspect of some embodiments of the present invention there is provided a pharmaceutical composition as described herein in any of the respective embodiments, for use in treating an autism spectrum disorder in a subject in need thereof.

According to an aspect of some embodiments of the present invention there is provided a method of treating neural cancer in a subject in need thereof, the method comprising administering to the subject an effective amount of a composition comprising an active agent which reduces nNOS activity, as described herein in any of the respective embodiments, wherein reduction in the nNOS activity is selectively or preferentially in a respective nerve tissue of the subject as compared to other tissues, thereby treating the neural cancer.

According to an aspect of some embodiments of the present invention there is provided a method of treating neural cancer in a subject in need thereof, the method comprising administering to the subject an effective amount of a pharmaceutical composition as described herein in any of the respective embodiments, thereby treating the cancer. According to some of these embodiments, the neural cancer is neuroblastoma.

According to an aspect of some embodiments of the present invention there is provided a pharmaceutical composition as described herein in any of the respective embodiments, for use in treating neural cancer in a subject in need thereof.

According to some of these embodiments, the agent that reduces nNOS activity is a compound of Formula I or a salt thereof, as described herein in any of the respective embodiments.

According to some of these embodiments, the neural cancer is neuroblastoma.

According to some of these embodiments, the agent that reduces nNOS activity is a compound of Formula I or a salt thereof, as described herein in any of the respective embodiments and the cancer is neuroblastoma.

According to some of any of the embodiments described herein, the agent is represented by Formula I as described herein in any of the respective embodiments,

According to an aspect of some embodiments of the present invention there is provided a method of producing a laboratory animal model for autism spectrum disorder (ASD), the method comprising administering to the laboratory animal an effective amount of a composition which increases NO levels in a brain of the animal to produce an ASD-like phenotype.

According to some of any of the embodiments described herein, the composition comprises an NO donor.

According to some of any of the embodiments described herein, the NO donor comprises S-nitroso-N-acetylpenicillamines (SNAP).

According to some of any of the embodiments described herein, the administering is systemic.

According to some of any of the embodiments described herein, the administering is directly into the brain of the animal.

According to some of any of the embodiments described herein, the NO donor is selected from the group consisting of SNAP, nitrates, nitrites, N-nitroso, C-nitroso, S-nitroso, heterocyclics, metal/NO complexes, diazeniumdiolates, S-nitrosothiols, sydnonimines and sodium nitroprusside (SNP).

According to an aspect of some embodiments of the present invention there is provided an animal model produced according to the method as described herein in any of the respective embodiments.

According to an aspect of some embodiments of the present invention there is provided a pharmaceutical compositions for the delivery of 7-nitroindazole to mammals

According to some embodiments of the invention, the pharmaceutical compositions as described herein is for the oral delivery of 7-nitroindazole.

According to some embodiments of the invention, the pharmaceutical composition as described herein is for delivery of 7-nitroindazole by injections for prolonged periods of time.

According to some embodiments of the invention, the pharmaceutical composition as described herein is a polymeric microparticles composition.

According to some embodiments of the invention, the polymer is PLGA.

According to some embodiments of the invention, the pharmaceutical composition as described herein is a pro-nanodispersion lipid formulation (PNL) or a dispersion of lipid particles in an aqueous solution formed therefrom.

According to some embodiments of the invention, the pharmaceutical composition as described herein comprises a solution or a mixture of surfactants, lipids and solvents that upon addition to aqueous media forms nanoparticles.

According to some embodiments of the invention, the pharmaceutical composition as described herein comprises microspheres composed of a biodegradable polymer.

According to some embodiments of the invention, the pharmaceutical composition as described herein comprises a composition administered subcutaneously or intramuscularly that possess continued, extended or slow release administration, over prolonged periods of time or targeted slow and regulated delivery.

According to some embodiments of the invention, the pharmaceutical composition as described herein is loaded in soft gelatin capsule for oral administration.

According to some embodiments of the invention, the pharmaceutical composition as described herein comprises microspheres dispersed in water for injection.

According to some embodiments of the invention, the pharmaceutical composition as described herein comprises the active agent (an agent that reduces nNOS activity as described herein) dispersed in a pasty biodegradable polymer carrier for injection.

According to an aspect of some embodiments of the present invention there is provided 7-nitoindazole salts with divalent and trivalent metal or ammonium counter-ions, as described herein.

According to an aspect of some embodiments of the present invention there is provided a method for treating autism spectrum disorder (ASD) comprising: administering to the subject in need of such treatment an effective amount of a neuronal NO-reducing composition.

According to some embodiments of the invention, the NO-reducing composition comprises one or more of a small molecule, an amino acid based molecule, or a nucleic acid based molecule (or a combination of several types of molecules, as described herein

According to some embodiments of the invention, the reduction in NO may be by one of the following mechanism:
Decrease in expression from the nNOS gene;
Inhibition of nNOS activity in the enzymatic level;
Decrease in the L-arginine level; and
Increase of GSNO reductase level or activity (decrease levels of GSNO).

According to an aspect of some embodiments of the present invention there is provided a method for producing an animal model for autism spectrum disorder the method comprising: administering to a laboratory animal an effective amount of a nitric oxide donor.

According to some embodiments of the invention, the administration is systemic

According to some embodiments of the invention, the administration is by a CNS targeted delivery system.

According to some embodiments of the invention, the administration is to the brain.

According to some embodiments of the invention, the administration is to the brain using nanoparticles delivered to the olfactory tissue.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patnet application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B present Western blot (WB) analysis (FIG. 1A) and a bar graph (FIG. 1B), showing an elevated level of Ntyr in blood samples of ASD kids compared to their typically developed (TD) counterparts. **P<0.01. Control (n=5) and ASD (n=10).

FIGS. 1C-E present Western blot (WB) analysis (FIG. 1C) and bar graphs (FIGS. 1C and 1D), of cortex tissues prepared from wild-type mice (C57BL/6 mice; WT1) and two ASD mouse models-SHANK3 mouse model (M1) and CNTNAP2 mouse model (M2). **P<0.01. Control (n=5) and ASD (n=S).

FIGS. 2A-B are bar graphs showing the effect of S-nitro-N-acetylpenicillamine (SNAP, an NO donor) treatment on WT mice, as determined in novel object recognition (NOR) test (FIG. 2A) and three-chambered sociability test (FIG. 2B). SNAP alters WT behavior. WT and SNAP mice were interested significantly in the familiar object (NOR) and empty cage (sociability test) compared to the WT with no treatments.

FIGS. 3A-B are bar graphs showing the effect of 7-NI treatment on mutant mice, as determined in NOR (FIG. 3A) and three-chambered sociability (FIG. 3B) tests. 7-NI reversed the autistic behavior in mutant mice.

FIGS. 3C-D present a Western blot (WB) analysis (FIG. 3C) and a quantitative bar graph (FIG. 3D), of cortex tissues prepared from wild-type mice (WT), SHANK3 model mice (M1) and SHANK3 model mice treated with 7-NI (M1+7-NI).

FIGS. 3E-F present a Western blot (WB) analysis (FIG. 3E) and a quantitative bar graph (FIG. 3F), of cortex tissues prepared from wild-type mice (WT), CNTNAP2^{(-/-)} model mice (M2) and CNTNAP2^{(-/-)} model mice treated with 7-NI (M2+7-NI).

FIGS. 3G-H present confocal microscopy images (FIG. 3G) and a quantitative bar graph (FIG. 3H) showing nitro-tyrosine in the cortical region of neurons of wild-type mice (WT), SHANK3 model mice (M1) and SHANK3 model mice treated with 7-NI (M1+7-NI).

FIGS. 4A-D present schematic illustrations of a novel object recognition test (NOR; FIG. 4A), a Three-chamber Sociability Test (FIG. 4B), Elevated Plus Maize Test (FIG. 4C), and Marble burying test (FIG. 4D).

FIGS. 5A-D show that SNAP administration induces ASD-like phenotypes in C57BL/6 mice, as determined in behavioral tests analysis for wild-type (WT) mice and SNAP-treated mice (i.p. injection of 20 mg/kg daily for 10 days). These results validate the results of the experiments shown in FIGS. 2A-B. FIG. 5A is a bar graph presenting data obtained in the novel object recognition test shown in FIG. 4A, showing object exploration time. The WT mice spent significantly more time exploring the novel object than the familiar one (left bars n=21, **P<0.01). The SNAP treated mice failed to display a significant preference neither for the novel object nor for the familiar one, indicating the lack of novelty seeking and interest (right bars, n=19, ns=not significant). FIG. 5B is a bar graph presenting data obtained in the Three-chamber sociability test shown in FIG. 4B, showing the (time of interacting with either a familiar mouse (S1) or a novel mouse (S2). The WT mice spent more time interacting with the novel mouse than the familiar one (left bars; n=31, *P<0.05), while the SNAP-treated mice did not show any significant preference to engage in social interaction with the novel mice (right bars, n=32, ns=not significant). FIG. 5C is a bar graph presenting data obtained in the Elevated plus-maze test shown in FIG. 4C, showing the time spent in the open arms. The SNAP-treated mice (n=16) exhibited significantly decreased time in the open arms compared to their WT counterparts (n=32, *P<0.05). FIG. 5D is a bar graph presenting data obtained in the Marble burying test shown in FIG. 4D, showing the number of marbles buried. The analysis showed that the WT mice buried more marbles than the SNAP-treated mice, indicating a lack of novelty seeking and restricted interests (n=20, 17 for the WT and WT+SNAP respectively. ***P<0.001). The data is presented as mean±SEM. Two-tailed t-test was conducted. *P<0.05.

FIGS. 6A-C present bar graphs showing the data obtained in a novel object recognition test (see, FIG. 4A) for wild-type (WT) mice, SHANK3 knockout mice (M1) and 7-NI-treated mice (M1+7NI) (i.p. injection of 80 mg/kg daily for 10 days) aged 6 weeks (FIG. 6A) and aged 10 months (FIGS. 6B and 6C). In FIG. 6A, it can be seen that the WT mice spent significantly more time exploring the novel object than the familiar one (n=21, **P<0.01). The M1 mice did not show any significant interest to explore neither the novel object nor the familiar one, indicating the lack of novelty seeking and interest (n=33, ns=not significant). The 7-NI-treated mice (M1+7NI) exhibited significantly increased time exploring the novel object than the familiar one (n=41, ***P<0.001). The data is presented as mean±SEM. Two-tailed t-test was conducted. *P<0.05, **P<0.01, ***P<0.001. Similar trends were observed in the aged mice, as can be seen in FIGS. 6B and 6C.

FIGS. 7A-B show data obtained in A three-chamber sociability test (FIG. 7A) and in an elevated plus maize test (FIG. 7B) for wild-type (WT) mice, the CNTNPA2 mutant mice (M2) and the 7-NI-treated mice (M2+7NI) (i.p. injection of 80 mg/kg daily for 10 days). 7-NI reversed the autistic phenotype in both tests.

FIGS. 8A-D are graphs showing behavioral test analyses of SHANK3 mutant mice that were treated with 7-NI at 0, 20,50, and 80 mg/kg. FIG. 8A presents the averaged velocity observed in an open field test. No significant difference was observed between the 80 mg/kg and the 20 and 50 mg/kg-treated mice. One-way ANOVA was conducted. ns=not significant. N=5. FIG. 8B presents the distance travelled observed in an open field test. No significant difference was observed in the total distance traveled between the 80, the 20 and the 50 mg/kg treated mice. One-way ANOVA was conducted. ns=not significant. N=5. FIG. 8C presents the time of interacting with a stranger mouse in the sociability test, first session. The 80 mg/kg-treated mice spent more time interacting with the stranger mouse compared to the mice that were treated with lower doses of 7-NI. One-way ANOVA was conducted. *P<0.05, **P<0.01. N=5. FIG. 8D presents the time of interacting with the novel stranger mouse (S2) in the sociability test. The 80 mg/kg treated mice spent more time interacting with the novel mouse compared to their counterparts that were treated with lower doses of 7-NI. One-way ANOVA was conducted ***P<0.001. N=5.

FIGS. 9A-D show a comparative Immunofluorescence analysis of synaptophysin and 3-nitrotyrosine in differentiated SH-SY-5Y cells, SH-SY-5Y+SHANK3 si RNA treatment, and SH-SY-5Y cells with SHANK3 siRNA+nNOS si-RNA group. Rabbit MAP2+primary mouse monoclonal 3-nitrotyrosine (FIGS. 9A-B). Primary rabbit monoclonal synaptophysin+primary mouse Map2 (FIGS. 9C-D). Cells were incubated with anti-mouse secondary Alexa fluor488 (green)/594 (red), and anti-rabbit secondary Alexa fluor 488 (green)/594 (red), conjugated secondary antibodies DAPI (Blue). The images shown in FIGS. 9A and 9C were acquired using a confocal microscope. The data presented in the gar graphs in FIGS. 9B and 9D are mean±SEM. One-way ANOVA followed by Fisher's LSD multiple comparison post hoc test was conducted. #P<0.05, **P<0.01, ***P<0.001.

FIGS. 10A-C present photographs of vials containing an exemplary pre-nanodispersion lipid (PNL) formulation without (MA-9-61-stock) and with (MA-9-61-A) 2% w/v 7-NI (FIG. 10A), photographs of vials containing aqueous dispersions of each formulation (FIG. 10B), and DLS particle size data of each formulation (FIG. 10C).

FIGS. 11A-F present exemplary synthetic pathways for preparing exemplary cationic salts of 7-NI (FIGS. 11A-E) and exemplary anionic salts of 7-NI (FIG. 11F).

FIGS. 12A-B present a representative UV spectra of 7-NI and a standard curve in methanol at 355 nm generated therefrom (FIG. 12A), and a plot showing the release of 7-NI from the PSARA gel formulation (MA-8-75-A) in phosphate buffer solution (PBS) at 37° C. The drug content in the formulation is 5% w/w.

FIGS. 13A-E show behavioral test analyses for wild-type (Control) mice, SHANK3 mutant mice (SHANK3) and the 7-NI-treated SHANK3 mice (Single administration—Treated) (SHANK3^{-/-}. i.p. injection of 5% w/w 7-NI PSARA gel formulation described in Example 12 herein). Experiments were initiated on the 4^{th} day after the injection. FIG. 13A presents data obtained in an open field test-motor activity (4^{th} day after the single administration). FIGS. 13B-C present the data obtained in the Three-chamber sociability test, on day1 (9^{th} day after the single administration) (FIG. 13B) and day2 (10^{th} day after the single administration) (FIG. 13C). The SHANK3 mice didn't show any significant change in the time interacting with the stranger1 mouse compared to the control mice. The 7-NI treated mice did spent significantly increased time interacting with the stranger 1 mouse compared to the untreated SHANK3 mice (FIG. 13B; n=4,4,4 for the Control, SHANK3, and treated mice respectively. ns=not significant, **P<0.01) The SHANK3 mice spent significantly less time interacting with the stranger 2 mouse compared to the control mice. The 7-NI-treated mice spent significantly increased time interacting with the stranger 2 mouse compared to the untreated SHANK3 mice. (FIG. 13C; n=4, 4, 4 for the Control, SHANK3, and treated, respectively. ***P<0.001. **P<0.01). The data obtained in the Marble burying test is presented in FIG. 13D (7^{th} day after the single administration), showing the number of marbles buried. The analysis showed that the SHANK3 mice buried significantly less marbles than the control mice, indicating a lack of novelty seeking and restricted interests. The 7-NI-treated mice buried more marbles comparing to SHANK3 mice (n=4,4,4 for the Control, SHANK3, and treated mice respectively. ***P<0.001, ***P<0.001). The data obtained in the Elevated plus maze test is presented in FIG. 13E (12^{th} day after the single administration). The SHANK3 mice didn't show any significant change in the time spent in the open arms compared to their control counterparts. The 7-NI-treated mice spent significantly more time in the open arms compared to their untreated littermates SHANK3. (n=4, 4, 4 for the Control, SHANK3, and treated, respectively. ns=not significant, ****P<0.05). The data is presented as mean±SEM. Two-tailed t-test was conducted. *P<0.05.

FIGS. 14A-E shows an optimal concentration of 7-NI (FIG. 14A), and successful knockdown of nNOS by siRNA (FIGS. 14B-E). FIG. 14A presents comparative plots showing the cell viability of SH-SY5Y as measured by MTT (the dose-response curve). The concentration of 7-NI for the cell treatment was 100 μM. Data were presented in percentage of 100. FIG. 14B is a representative blot of nNOS. 1, SH-SY5Y; 2, SH-SY5Y+negative control (NC); and 3, SH-SY5Y+si-nNos. FIG. 14C presents representative fluorescence images of nNOS immunofluorescence. Blue color represents DAPI (a marker of nucleus), green color shows NeuN (a marker of neuron) and red color represents nNOS. FIG. 14D is a bar graph showing the relative abundance of nNOS. FIG. 14E is a bar graph showing the relative fluorescence intensity of nNOS.

FIGS. 15A-B are bar graphs showing the effects of 7-NI and nNOS silencing on the nNOS activity and NO levels in SH-SY5Y cells. FIG. 15A shows NADPH-diaphorase activity in cell lysates. The absorbance at 585 nm was normalized for total protein. FIG. 15B shows nitrite levels (a marker of NO levels) measured by Griess assay. Data are expressed as the mean±SEM (n=3) *P<0.05, **P<0.01.

FIGS. 16A-B show the effects of 7-NI and nNOS silencing on SH-SY5Y cell proliferation (the clonogenic growth assay). FIG. 16A presents representative images of plates with SH-SY5Y cells non-treated and treated with 7-NI, si-nNOS RNA (silenced nNOS), and vehicle (negative control, NC). FIG. 16B is a bar graph showing the number of cell colonies. Data are expressed as the mean±SEM (n=3) *P<0.05, **P<0.01.

FIGS. 17A-D present data obtained in Western blot analysis of the levels of 3-nitrotyrosine, components of the mTOR signaling pathway, and synaptophysin. Groups of cells: SH-SY5Y, SH-SY5Y+7-NI, and SH-SY5Y+si-nNOS (nNOS knockdown); n=9 for each group. FIG. 17A presents a representative WB of protein prepared from cell lysate of SH-SY5Y (1), SH-SY5Y+7-NI (2), and SH-SY5Y+si-nNOS (3). FIG. 17B is a bar graph showing the relative abundance of 3-Ntyr in the 3 groups of cells. FIG. 17C presents a representative Western blotting (WB) of proteins (p-mTOR, mTOR, TSC2, pAKT, AKT, pRPS6, RPS6, and Syp) prepared from the cell lysate of the 3 groups. FIG. 17D are bar graphs showing the relative abundance of p-mTOR, mTOR, TSC2, pAKT, AKT, pRPS6, RPS6, and Syp. The data were normalized for β-actin and presented as mean±SEM. A one-way ANOVA followed by Fisher's LSD multiple comparison post hoc test was conducted. *P<0.05, **P<0.01.

FIGS. 18A-C show immunofluorescence of synaptophysin and 3-nitrotyrosine in SH-SY5Y cells with inhibited nNOS activity. Cells (with or without treatment) were postfixed in 4% paraformaldehyde and then incubated with the primary rabbit monoclonal Syp (red), and either primary mouse monoclonal NeuN (green), or primary mouse monoclonal 3-Ntyr (green) antibodies, in another group of cells. Cells were incubated with their respective secondary antibodies, such as anti-mouse Alexa fluor488, or anti-rabbit Alexa fluor594, and then mounted with DAPI (Blue) in both groups of cells. The images were acquired using a confocal microscope. Groups of cells: SH-SY5Y, SH-SY5Y+7-NI, and SH-SY5Y+si-nNOS. Each group consisted of three independent sets of experiments and was run in triplicates. The initial cell seed number was ˜10⁶/cm². FIG. 18A presents representative images of Syp and 3-Ntyr immunofluorescence. FIGS. 18B-C are bar graphs showing the relative fluorescent intensity of Syp (FIG. 18B) and N-Tyr (FIG. 18C) immunofluorescence in the 3 groups of cells. The data are presented as mean±SEM. One-way ANOVA followed by Fisher's LSD multiple comparison post hoc test was conducted. *P<0.05, **P<0.01, ***P<0.001. The scale bar is 100 μm.

### DESCRIPTION OF SPECIFIC EMBODIMENTS OF THE INVENTION

The present invention, in some embodiments thereof, relates to therapy, and, more particularly, but not explicitly, to pharmaceutical compositions and methods for treating neurological conditions associated with abnormal activity of neuronal nitric oxide synthase (nNOS) such as, but not limited to, autistic spectrum disorders (ASDs) and neural-derived cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have recognized that neurological conditions such as, for example, ASD, Alzheimer's disease and neuroblastoma are featured by a significant elevation of nitric oxide (NO) (e.g., free, physiologically available NO), specifically in the neurons, and have surprisingly uncovered that treatment with nNOS inhibitors leads to reversal in the diseased phenotype, thereby demonstrating a role for inhibition or reduction in nNOS activity in treating neurological conditions that are associated with elevated NO levels.

The present inventors have designed pharmaceutical compositions that are usable in the inhibiting nNOS activity and in treating neurological conditions that are associated with elevated NO levels and/or aberrant nNOS activity.

Embodiments of the present invention relate to methods for reducing a level (e.g., amount or concentration) of physiologically available nitric oxide and/or for reducing an activity of nNOS in a subject in need thereof.

Embodiments of the present invention relate to methods method of treating a disease or condition in which a beneficial clinical effect is achieved by reduction in nNOS activity, such as, but not limited to, ASD or cancer of a neural original (neural-derived cancer or neural cancer).

Embodiments of the present invention further relate to pharmaceutical compositions usable in any of the above methods.

Some embodiments of the present invention relate to pharmaceutical compositions and/or methods in which an active agent that reduces an activity of nNOS reduces the nNOS activity selectively or preferentially in the CNS of the subject (e.g., the brain).

Some embodiments of the present invention relate to pharmaceutical compositions that comprises an agent is a selective nNOS inhibitor.

### Methods and Uses

According to an aspect of some embodiments of the invention, there is provided a method of treating a disease or condition in which a beneficial clinical effect is achieved by reduction in nNOS activity, the method comprising administering to a subject in need thereof an effective amount of a composition comprising an active agent which reduces nNOS activity, thereby treating the disease or condition in which a beneficial clinical effect is achieved by reduction in nNOS activity.

According to an aspect of some embodiments of the present invention there is provided a composition comprising an active agent which reduces nNOS activity, and optionally a pharmaceutically acceptable carrier, for use in treating the disease or condition in which a beneficial clinical effect is achieved by reduction in nNOS activity.

According to some of any of the embodiments described herein, the methods and uses as described herein are for reducing a level (e.g., an amount) of physiologically available nitric oxide (NO) in a neuronal cells of a subject in need thereof. According to some embodiments, the reduction is by at least 20%, or at least 30% or at least 40%, or at least 50%.

According to some of any of the embodiments described herein, the methods and uses as described herein are for reducing a level (e.g., an amount) of physiologically available nitric oxide (NO) in the CNS (e.g., the brain) of a subject in need thereof. According to some embodiments, the reduction is by at least 20%, or at least 30% or at least 40%, or at least 50%.

According to an aspect of some embodiments of the invention, there is provided a method of treating a disease or condition in which a beneficial clinical effect is achieved by reduction in an amount of physiologically available nitric oxide in a diseased tissue, the method comprising administering to a subject in need thereof an effective amount of a composition comprising an active agent which reduces nNOS activity, thereby treating the disease or condition. According to some embodiments, an effective amount of the composition is an amount that results in reducing an amount of the physiologically available nitric oxide by at least 20%, or at least 30%, or at least 50%, or at least 60%, or at least 70%. According to some embodiments, the diseased tissue comprise neuronal cell. According to some embodiments, the diseased tissue is in the CNS (e.g., brain) of the subject.

Reducing an activity of nNOS or reducing an amount of physiologically available nitric oxide can be measured by determining an amount of Ntyr in the plasma of the subject, or by any other methods known in the art, as discussed in further detail hereinafter.

According to some of any of the embodiments described herein, the methods and uses as described herein are for reducing nNOS activity in the CNS, or in the brain.

According to some of any of the embodiments described herein, the methods and uses as described herein are for reducing an amount of physiologically available nitric oxide in neuronal cells, for example, in the CNS or in the brain.

According to an aspect of some embodiments of the invention, there is provided a method of treating a disease or condition in which a beneficial clinical effect is achieved by reduction in nNOS activity in the brain, the method comprising administering to a subject in need thereof an effective amount of a composition comprising an active agent which reduces nNOS activity, wherein reduction in said nNOS activity is selectively or preferentially in the brain of the subject as compared to other tissues, thereby treating the disease or condition in which a beneficial clinical effect is achieved by reduction in nNOS activity in the brain.

Alternatively or additionally, there is provided a composition comprising an active agent which reduces nNOS activity for use in treating a disease or condition in which a beneficial clinical effect is achieved by reduction in nNOS activity in a brain of a subject in need thereof, wherein the composition is formulated such that a reduction in said nNOS activity is selectively or preferentially in the brain of the subject as compared to other tissues.

As used herein, the terms “treating” and “treatment” include abrogating, substantially inhibiting, slowing or reversing the progression of a condition, substantially ameliorating clinical or aesthetical symptoms of a condition or substantially preventing the appearance of clinical or aesthetical symptoms of a condition.

According to a specific embodiment, the term “treatment” can refer to the reduction of the severity or duration of the undesired behaviors, increasing the duration between boosts of undesired behaviors, preventing the behaviors before they occur, improvement in development and improved overall condition.

According to a specific embodiment, the terms “treatment” and “treating” exclude prevention.

As used herein “a disease or condition in which a beneficial clinical effect is achieved by reduction in nNOS activity” refers to a medical condition in which elevated levels of nitric oxide (NO) are evident, as compared to NO levels in a control subject who doesn't suffer from the medical disease or condition. According to a specific embodiment, the control subject belongs to a control population of the same gender, developmental stage and ethnic group, as that of the treated subject.

As used herein “a disease or condition in which a beneficial clinical effect is achieved by reduction in nNOS activity in the brain” refers to a medical condition in which elevated levels of nitric oxide (NO) are evident in a part of the central nervous system (CNS), as compared to NO levels in a control subject who doesn't suffer from a medical condition in the CNS. According to a specific embodiment, the control subject belongs to a control population of the same gender, developmental stage and ethnic group, as that of the treated subject

As used herein “nNOS” refers to the protein product of the NOS1 gene located on chromosome 12 in human beings, 12q24.22, BRENDA 1.14.13.39. It is also known as NOS1, an enzyme that in humans is encoded by the NOS1 gene. The nNOS gene is depicted in humans under accession number P29475.

Orthologs from other non-human animals are also contemplated herein. Nitric oxide synthases (EC 1.14.13.39) (NOSs) are a family of synthases that catalyze the production of nitric oxide (NO) from L-arginine. NO is a chemical messenger with diverse functions throughout the body depending on its enzymatic source and tissue localization. In the brain and peripheral nervous system, where NOS1 is largely present, NO displays many properties of a neurotransmitter and may be involved in long term potentiation.

Neuronal NOS (NOS1), Endothelial NOS (NOS3), and Inducible NOS macrophage NOS are distinct isoforms. Both the neuronal and the macrophage forms are unusual among oxidative enzymes in requiring several electron donors (co-factors), including, for example: flavin adenine dinucleotide (FAD), flavin mononucleotide (FMN), NADPH, and tetrahydrobiopterin. All nitric oxide synthase isoforms are calcium-calmodulin controlled. For example, nNOS is activated at calcium concentrations over 100 nM.

As used herein “nNOS activity” refers to the production of free (physiologically available) nitric oxide (NO) in neuronal cells or in a nerve tissue (neuronal tissue), including, unless otherwise indicated, the peripheral and/or the central nervous system.

“nNOS activity” can also be referred to as the production of free (physiologically available) nitric oxide (NO) by nNOS enzyme in a nerve tissue (CNS, e.g., brain) or peripheral organs which comprise neuronal cells that express nNOS.

The production of NO by NOS is effected by catalyzing a five-electron oxidation of a nitrogen atom of the guanidine group of L-arginine. Oxidation of L-Arg to L-citrulline occurs via two successive monooxygenation reactions, which produce N^{ω}-hydroxy-L-arginine (NOHLA) as an intermediate. 2 mol of O₂ and 1.5 mol of NADPH are consumed per mole of NO formed.

nNOS activity can be effected (increased or reduced) by interfering in the production of free NO, by means of, for example, affecting a level of one or more co-factors that participate in the monooxygenation reactions and/or a level of its substrate (L-arginine) and/or by interfering with a level of its binding to L-arginine, for example, by means of substrate-competitive inhibitors.

Uncontrolled production of free NO by nNOS (increased as compared to a healthy state) may elicit ASD like symptoms in a model animal such as described hereinbelow and in the Examples section which follows.

Reduction in nNOS activity can be achieved by down-regulating the level of the nNOS protein product, DNA or mRNA encoding the same, or by inhibiting the activity of the enzyme. This can be achieved by interfering with the cellular localization of the enzyme, inhibiting its intrinsic activity or inhibiting an activity of an activator or an effector thereof, wherein each alternative is considered as an individual embodiment of the invention.

For example, inhibiting nNOS activity can be effected by interfering in the production of free NO, by means of, for example, reducing a level of one or more co-factors that participate in the monooxygenation reactions and/or reducing a level of its substrate (L-arginine) and/or by interfering with a level of its binding to L-arginine, for example, by means of substrate-competitive inhibitors.

Selective inhibition of nNOS, compared to inhibition of other NOS isoforms, can be effected by interfering with a level of a co-factor that participates in the nNOS-catalyzed NO production but not in the NO production that is catalyzed by other isoforms, and/or by using a substrate-competitive inhibitor that has an affinity to nNOS that is higher compared to its affinity to the other NOS isoforms. In some embodiments, a selective nNOS inhibitor has an affinity to nNOS, determined by the dissociation constant (Ki) for nNOS that is higher by at least 10-folds or at least 100-folds or at least 1,000-folds than its dissociation constant (Ki) for eNOS or iNOS.

Nitric oxide synthase activity assays are commercially available. These include, for example, the NOS activity assay kit by Abcam e.g., ab211083. An activity parameter may be Tyrosine nitration, NO₂, NO₃, and others.

Alternatively, or additionally, nNOS levels can be determined at the protein or mRNA levels.

nNOS protein levels can be determined using an immunological assay, e.g., Western blotting, ELISA, such as for example, by using an ELISA assay kit such as that available from Cosmo Bio USA or LSBio e.g., LS-F4243.

Dotsch et al. Int J Cancer. 2000 Oct. 15; 88 (2):172-5. teach determination of nNOS at the mRNA level.

As used herein “a disease or condition in which a beneficial clinical effect is achieved by reduction in nNOS activity” refers to a medical condition which onset or progression is dependent on over production (as compared to its level in a normal/healthy CNS tissue) of nitric oxide (NO) in neuronal cells. This is typically achieved by dysregulated (increased) activity in nNOS as compared to a healthy nerve tissue (of the peripheral or central nervous system).

In some embodiments, “a disease or condition in which a beneficial clinical effect is achieved by reduction in nNOS activity” refers to a medical condition which onset or progression is dependent on over production (as compared to its level in a normal/healthy CNS tissue) of nitric oxide (NO) in the CNS (e.g., brain). This is typically achieved by dysregulated (increased) activity in nNOS as compared to a healthy CNS.

A beneficial effect relates to a decrease in at least one pathological symptom.

As used herein “decrease in at least one symptom associated with the disease or condition” refers to a statistically significant decrease, e.g., at least 10%, 20%, 30%, 40%, 50%, 60%, 70%, 80%, 90% or even complete disappearance of the symptom.

A skilled artisan would know how to determine the level of a symptom, or presence or absence thereof.

According to a specific embodiment, reduction/decrease in nNOS activity (or expression) is optimally to its level or a level of NO in a normal/healthy tissue of the CNS or to the level that improves the medical condition of the patient.

Exemplary diseases or conditions according to the present embodiments include, but are not limited to, Alzheimer's disease, Parkinson's disease, Huntington's disease, Multiple sclerosis, Schizophrenia, addiction, ALS, Epilepsy, bipolar disease, migraine and in addition all kinds of neurodevelopmental disorders as well as neurodegenerative diseases associated with unbalanced NO levels. Additional examples include, but are not limited to, depression, ADD, ADHD, as well as hypoxic and ischemic conditions of the CNS such as trauma, hypoxic-ischemic encephalopathy of a newborn, during or after surgery; metabolic diseases involving the central nervous system such as Sphingolipid metabolism diseases (i.e., GM1-gangliosidosis. GM2-gangliosidoses, Tay-Sachs disease, Sandhoff disease, AB-variant of GM2-gangliosidosis, Fabry disease, Gaucher disease, Metachromatic leukodystrophy, Krabbe disease, Niemann-Pick disease, type A, B, C or Farber disease). See Tripathi M K, Kartawy M and Amal H. The role of nitric oxide in brain disorders: Autism spectrum disorder and other psychiatric, neurological, and neurodegenerative disorders. Redox Biol. 2020:101567, which is incorporated by reference as if fully set forth herein.

The present inventors have shown an alleviation in ASD-like symptoms in mouse models treated with a nNOS inhibitor as shown the Examples section which follows.

According to a specific embodiment, the disease or condition is a brain disorder.

According to a specific embodiment, the disease or condition is a memory or mood disorder.

According to a specific embodiment, the disease or condition is a neuropsychiatric disorder.

According to a specific embodiment, the disease or condition is an autism spectrum disorder (ASD).

According to a specific embodiment, the term “autistic spectrum disorder” refers to conditions previously diagnosed as autism (autistic disorder), including high-functioning autism, Asperger syndrome, pervasive developmental disorder not otherwise specified (PDD-NOS), and childhood disintegrative disorder.

The term “autistic spectrum disorder” or ASD also encompasses other disease or medical conditions that involve ASD symptoms (e.g., impaired communication and social interaction), as defined, for example, hereinbelow, including, for example, ADD, ADHD, ADNP (Helsmoortel-Van Der Aa) syndrome and NAP (Davunetide) syndrome, and conditions associated therewith (e.g., Alzheimer's disease).

The term “treatment of ASD” refers to improvement in at least one undesired parameter, characterization, behavior associated with ASD including: problems with social communication and social interaction, the presence of restricted, repetitive patterns of behavior, interests, or activities, Self-injurious behavior (SIB), abnormal responses to sensations including sights, sounds, touch, and smell, problems keeping a consistent speech rhythm, developmental problems manifested in perceptual disturbances, disturbances of development rate, relating, speech and language, and motility, and including also memory deficiency and other cognitive malfunctions.

According to a specific embodiment, the ASD is defined according to DSM-5, as provided infra.

A. Persistent deficits in social communication and social interaction across multiple contexts, as manifested by the following, currently or by history (examples are illustrative, not exhaustive):

1. Deficits in social-emotional reciprocity, ranging, for example, from abnormal social approach and failure of normal back-and-forth conversation; to reduced sharing of interests, emotions, or affect; to failure to initiate or respond to social interactions.

2. Deficits in nonverbal communicative behaviors used for social interaction, ranging, for example, from poorly integrated verbal and nonverbal communication; to abnormalities in eye contact and body language or deficits in understanding and use of gestures; to a total lack of facial expressions and nonverbal communication.

3. Deficits in developing, maintaining, and understanding relationships, ranging, for example, from difficulties adjusting behavior to suit various social contexts; to difficulties in sharing imaginative play or in making friends; to absence of interest in peers.

Severity is based on social communication impairments and restricted repetitive patterns of behavior. (See Table 1 below.)

B. Restricted, repetitive patterns of behavior, interests, or activities, as manifested by at least two of the following, currently or by history (examples are illustrative, not exhaustive):

1. Stereotyped or repetitive motor movements, use of objects, or speech (e.g., simple motor stereotypies, lining up toys or flipping objects, echolalia, idiosyncratic phrases).

2. Insistence on sameness, inflexible adherence to routines, or ritualized patterns or verbal nonverbal behavior (e.g., extreme distress at small changes, difficulties with transitions, rigid thinking patterns, greeting rituals, need to take same route or eat food every day).

3. Highly restricted, fixated interests that are abnormal in intensity or focus (e.g., strong attachment to or preoccupation with unusual objects, excessively circumscribed or perseverative interest).

4. Hyper- or hypo-reactivity to sensory input or unusual interests in sensory aspects of the environment (e.g., apparent indifference to pain/temperature, adverse response to specific sounds or textures, excessive smelling or touching of objects, visual fascination with lights or movement).

Severity is based on social communication impairments and restricted, repetitive patterns of behavior. (See Table A below.)

C. Symptoms must be present in the early developmental period (but may not become fully manifest until social demands exceed limited capacities or may be masked by learned strategies in later life).

D. Symptoms cause clinically significant impairment in social, occupational, or other important areas of current functioning.

E. These disturbances are not better explained by intellectual disability (intellectual developmental disorder) or global developmental delay. Intellectual disability and autism spectrum disorder frequently co-occur; to make comorbid diagnoses of autism spectrum disorder and intellectual disability, social communication should be below that expected for general developmental level.

Individuals with a well-established DSM-IV diagnosis of autistic disorder, Asperger's disorder, or pervasive developmental disorder not otherwise specified should be given the diagnosis of autism spectrum disorder. Individuals who have marked deficits in social communication, but whose symptoms do not otherwise meet criteria for autism spectrum disorder, should be evaluated for social (pragmatic) communication disorder.

Specify if:
With or without accompanying intellectual impairment;
With or without accompanying language impairment;
(Coding note: Use additional code to identify the associated medical or genetic condition.)
Associated with another neurodevelopmental, mental, or behavioral disorder;
(Coding note: Use additional code[s] to identify the associated neurodevelopmental, mental, or behavioral disorder[s].)
With catatonia
Associated with a known medical or genetic condition or environmental factor;

TABLE A
Severity levels for autism spectrum disorder

| Severity | | |
|---|---|---|
| level | Social communication | Restricted, repetitive behaviors |
| Level 3 | Severe deficits in verbal and | Inflexibility of behavior, extreme |
| “Requiring | nonverbal social communication skills | difficulty coping with change, or other |
| very | cause severe impairments in | restricted/repetitive behaviors |
| substantial | functioning, very limited initiation of | markedly interfere with functioning in |
| support” | social interactions, and minimal | all spheres. Great distress/difficulty |
| | response to social overtures from | changing focus or action. |
| | others. For example, a person with | |
| | few words of intelligible speech who | |
| | rarely initiates interaction and, when | |
| | he or she does, makes unusual | |
| | approaches to meet needs only and | |
| | responds to only very direct social | |
| | approaches | |
| Level 2 | Marked deficits in verbal and | Inflexibility of behavior, difficulty |
| “Requiring | nonverbal social communication | coping with change, or other |
| substantial | skills; social impairments apparent | restricted/repetitive behaviors appear |
| support” | even with supports in place; limited | frequently enough to be obvious to the |
| | initiation of social interactions; and | casual observer and interfere with |
| | reduced or abnormal responses to | functioning in a variety of contexts. |
| | social overtures from others. For | Distress and/or difficulty changing |
| | example, a person who speaks simple | focus or action. |
| | sentences, whose interaction is | |
| | limited to narrow special interests, and | |
| | how has markedly odd nonverbal | |
| | communication. | |
| Level 1 | Without supports in place, deficits in | Inflexibility of behavior causes |
| “Requiring | social communication cause | significant interference with |
| support” | noticeable impairments. Difficulty | functioning in one or more contexts. |
| | initiating social interactions, and clear | Difficulty switching between activities. |
| | examples of atypical or unsuccessful | Problems of organization and planning |
| | response to social overtures of others. | hamper independence. |
| | May appear to have decreased interest | |
| | in social interactions. For example, a | |
| | person who is able to speak in full | |
| | sentences and engages in | |
| | communication but whose to- and-fro | |
| | conversation with others fails, and | |
| | whose attempts to make friends are | |
| | odd and typically unsuccessful. | |

According to a specific embodiment, the condition is Social (Pragmatic) Communication Disorder. Diagnostic criteria according to DSM-5 are summarized infra.

A. Persistent difficulties in the social use of verbal and nonverbal communication as manifested by all of the following:

1. Deficits in using communication for social purposes, such as greeting and sharing information, in a manner that is appropriate for the social context.

2. Impairment of the ability to change communication to match context or the needs of the listener, such as speaking differently in a classroom than on the playground, talking differently to a child than to an adult, and avoiding use of overly formal language.

3. Difficulties following rules for conversation and storytelling, such as taking turns in conversation, rephrasing when misunderstood, and knowing how to use verbal and nonverbal signals to regulate interaction.

4. Difficulties understanding what is not explicitly stated (e.g., making inferences) and nonliteral or ambiguous meanings of language (e.g., idioms, humor, metaphors, multiple meanings that depend on the context for interpretation).

B. The deficits result in functional limitations in effective communication, social participation, social relationships, academic achievement, or occupational performance, individually or in combination.
C. The onset of the symptoms is in the early developmental period (but deficits may not become fully manifest until social communication demands exceed limited capacities).
D. The symptoms are not attributable to another medical or neurological condition or to low abilities in the domains or word structure and grammar, and are not better explained by autism spectrum disorder, intellectual disability (intellectual developmental disorder), global developmental delay, or another mental disorder.

Each of ASD and SCD are contemplated herein as co-morbidities or alternative embodiments.

The following co-occurrences are also contemplated each of which is considered as an alternative embodiment.

Genetic disorders. About 10-15% of autism cases have an identifiable Mendelian (single-gene) condition, chromosome abnormality, or other genetic syndrome, and ASD is associated with several genetic disorders [Zafeiriou D I, Ververi A, Vargiami E (2007). “Childhood autism and associated comorbidities”. Brain Dev. 29 (5): 257-272. doi: 10.1016/j.braindev.2006.09.003].

Intellectual disability. The percentage of autistic individuals who also meet criteria for intellectual disability has been reported as anywhere from 25% to 70% [Dawson et al. Learning and Memory: A Comprehensive Reference. Vol. 2. Elsevier. pp. 759-772. doi: 10.1016/B978-012370509-9.00152-2. ISBN 978-O-12-370504-4. OCLC 775005136].

Anxiety disorders are common among children with ASD, with prevalences ranging from 11% to 84% [White et al. 2009 Clin Psychol Rev. 29 (3): 216-229].

Epilepsy, with variations in risk of epilepsy due to age, cognitive level, and type of language disorder [Spence et al. ediatr Res. 65 (6): 599-606. doi: 10.1203/PDR.0b013e31819e7168].

Several metabolic defects, such as phenylketonuria, are associated with autistic symptoms [Manzi et al. J Child Neurol. 23 (3): 307-314. doi: 10.1177/0883073807308698].

Attention deficit hyperactivity disorder (ADHD), Tourette syndrome, Attention deficit disorder (ADD) and other of these conditions are often present and these co-occurrent conditions are increasingly accepted.

Sleep problems affect about two-thirds of individuals with ASD at some point in childhood. These most commonly include symptoms of insomnia such as difficulty in falling asleep, frequent nocturnal awakenings, and early morning awakenings.

According to a specific embodiment, the disease or condition is selected from the group consisting of Alzheimer's disease, Parkinson's disease, Huntington's disease, multiple sclerosis, schizophrenia, addiction, amyotrophic lateral sclerosis (ALS), epilepsy, bipolar disease and migraine.

According to a specific embodiment, the disease or condition is a neurodevelopmental disorder, a psychiatric disorder or a neurodegenerative disease.

NO takes part in the development of cancers. The present inventors have shown that inhibition of NO production in neuroblastoma (NB) cells, either by pharmacological intervention, using the selective nNOS inhibitor 7-NI, or by genetic ablation, using a specific gene silencing tool for silencing expression of nNOS, is able to suppress NB malignancy. This effect was manifested by reduced proliferation, significantly reduced levels of the marker of NB Syp (MIETTINEN and RAPOLA, 1987), and nitrosative stress, seen by the reduced levels of 3-Ntyr. The present inventor has further shown, for the first time, that the pro-cancer effect of nNOS is mediated by the activation of the mTOR signaling pathway and that the selective nNOS inhibitor 7-NI is able to inhibit significantly both the activation of the mTOR signaling and malignancy of the human NB cells. These results suggest that the nNOS-mTOR axis could serve as a novel potential target for NB treatment.

According to a specific embodiment, the disease or condition is a cancer of a neural origin or a neuroectodermal origin, which is also referred to herein as neural cancer or as neural-derived cancer.

According to a specific embodiment, the cancer is selected from the group consisting of neuroblastoma, glioma, ganglioglioma, central neurocytoma, ganglioneuroblastoma, medulloblastoma and a primitive neuroectodermal tumor (PNET).

According to a specific embodiment, the cancer is neuroblastoma.

According to a specific embodiment, the neuroblastoma comprises a mutation in ALK. The tyrosine kinase receptor anaplastic lymphoma kinase (ALK) can be abnormally activated in neuroblastoma, and somatic ALK mutations occur in 6%-10% of patients.

According to a specific embodiment, the tumor is a primary tumor.

According to a specific embodiment, the tumor is a metastatic tumor.

According to a specific embodiment, the tumor is a tumor metastasis.

According to a specific embodiment, the tumor is a recurrent tumor.

According to a specific embodiment, the tumor is resistant to a first-line (e.g., chemotherapy) treatment.

Active Agents (an Agent that Reduced nNOS Activity):

Regardless of the medical condition to be treated, embodiments of the invention relate to administration of an effective amount of a composition comprising an active agent which reduces nNOS activity, as defined herein. In some embodiments, the active agent reduces an amount of physiologically available NO in neuronal cells, as described herein.

According to a specific embodiments, the reduction in the nNOS activity is selectively or preferentially in neuronal cells or a nerve tissue of the subject as compared to other tissues.

According to a specific embodiments, the reduction in the nNOS activity is selectively or preferentially in a peripheral and/or central nervous system of the subject as compared to other tissues.

According to a specific embodiments, the reduction in the nNOS activity is selectively or preferentially in a brain of the subject as compared to other tissues.

Alternatively or additionally there is provided a pharmaceutical composition comprising an active agent which reduces nNOS activity and a pharmaceutically acceptable carrier, wherein the composition and the active agent are such that upon administration to a subject, reduction in the nNOS activity is effected selectively or preferentially in a nerve tissue of the subject as compared to other tissues, as described herein.

Such a composition is also referred to herein as “nNOS-reducing composition”, or, alternatively, as “NO-reducing composition”.

As used herein “an active agent which reduces nNOS activity” refers to a substance or a physical condition which reduces the activity of nNOS, by any one of: affecting its inherent catalytic activity, ability to interact with other cellular proteins or factors, affecting its cellular localization and/or affecting its level or level of NO within the cell.

As mentioned, in some embodiments, the agent (which is also referred to as an “nNOS inhibitor” or a “neuronal NO reducing composition”) is acting such that the reduction in nNOS activity is selectively or preferentially achieved in neuronal cells or a nerve tissue to be treated, for example, the CNS (e.g., brain) of the subject, as compared to other tissues. nNOS is expressed in various tissues, for example, brain, retina, frontal cortex, heart, colon, colon muscle, rectum, prostate and pancreas.

Alternatively or additionally, according to some embodiments of the invention, the agent is active on nNOS and does not affect the activity of other NOS enzymes such as NOS2 and/or NOS3, as described hereinabove with respect to nNOS selectivity.

Any of these embodiments, individually or combined, ensure activity of the agent only in the affected site (or predisposed site), without acting on other NOS (e.g., iNOS) enzymes or in other tissues in which nNOS is expressed.

According to an embodiment, the agent decreases nNOS expression.

According to an embodiment, the agent decreases nNOS translation.

According to an embodiment, the agent inhibits nNOS enzymatic activity.

According to a specific embodiment the agent affects nNOS interaction with other proteins or its dimerization. For example, the protein acts as a homodimer. It interacts with DLG4; the interaction possibly being prevented by the association between NOS1 and CAPON. Forms a ternary complex with CAPON and RASD1. Forms a ternary complex with CAPON and SYN1. Interacts with ZDHHC23. Interacts with NOSIP; which may impair its synaptic location (By similarity). Interacts with HTR4. Interacts with VAC14 (By similarity). Interacts with SLC6A4 (By similarity). Interacts (via N-terminal domain) with DLG4 (via N-terminal tandem pair of PDZ domains).

According to some of any of the embodiments described herein, the active agent in the composition may be a small molecule, an amino acid based molecule (peptide, protein, antibody or RNA encoding same), or a nucleic acid based molecule (e.g., RNA including antisense and inhibitor RNA; DNA) or a combination of several types of molecules.

It will be appreciated that when RNA encoding the claimed proteinaceous agent is used, measures are taken to improve its stability and bioavailability as described for instance in U.S. 20150030576.

As mentioned, the reduction in the amount of free NO may be by one of the following mechanism:

1. Decrease in expression of the nNOS from its gene (in the transcription or translation level), or at the DNA level such as achieved by genome editing.

2. Inhibition of nNOS activity in the enzymatic level (by small molecule, competing peptide, antibody, antibody fragment)

3. Decrease in the L-arginine levels, the precursor for NO. Using targeted mass spectrometry for small molecule (Triple quad), one can measure accurate concentrations of arginine

4. Increase of GSNO reductase level or activity (decrease levels of GSNO). By measuring protein expression using WB and measuring its activity using ELISA, we can conclude on the level and activity.

As used herein the phrase “decreases expression or activity” or “downregulates expression or activity” (interchangeably used) refers to downregulating the expression of a protein (e.g. nNOS) at the genomic (e.g. homologous recombination and site specific endonucleases) and/or the transcript level using a variety of molecules which interfere with transcription and/or translation (e.g., RNA silencing agents) or on the protein level (e.g., aptamers, small molecules and inhibitory peptides, antagonists, enzymes that cleave the polypeptide, antibodies and the like).

According to a specific embodiment, the nNOS gene is depicted under accession number P29475. According to an embodiment of the invention the expression o from the gene may be selectively reduced at the transcription or translation level

For the same culture conditions the expression or activity is generally expressed in comparison to the expression or activity in a cell of the same species but not contacted with the agent or contacted with a vehicle control, also referred to as control.

Down regulation of expression may be either transient or permanent.

According to specific embodiments, down regulating expression refers to the absence of mRNA and/or protein, as detected by RT-PCR or Western blot, respectively.

Primers for detecting nNOS expression are well within the capabilities of the skilled artisan and some of which are described hereinbelow, in the Examples section which follows.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down-regulating nNOS expression are described in details hereinbelow.

### Nucleic Acid Agents:

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

According to specific embodiments, the downregulating agent is a polynucleotide capable of hybridizing to a gene or mRNA encoding nNOS.

According to specific embodiments, the downregulating agent directly interacts with nNOS.

According to specific embodiments, the agent directly binds nNOS.

According to specific embodiments, the agent indirectly binds nNOS (e.g. binds an effector of nNOS). More specifically, a nNOS inhibitor (e.g., 7-NI) leads to de-nitrosylation of TSC2 which leads to a reduction in mTOR signaling.

According to specific embodiments the downregulating agent is an RNA silencing agent or a genome editing agent.

Thus, downregulation of nNOS can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., nNOS) and does not cross inhibit or silence other targets (e.g., (iNOS or eNOS) or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects-see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13 (5): 381-392. doi: 10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC. Exemplary siRNAs which can be used in accordance with the present teachings are provided in the Examples section which follows and in SEQ ID NOs: 3-8.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

Exemplary siRNAs which can be used in accordance with the present teachings are provided in the Examples section which follows and in SEQ ID NOs: 9-14.

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ (SEQ ID NO: 1) and 5′-UUACAA-3′ (SEQ ID NO: 2) (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the nNOS mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912.html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

According to a specific embodiment, the miRNA is miR-146a or synthetic version thereof. Other contemplated miRS include, but are not limited to, hsa-miR-146a-5p (MIRT735124), hsa-miR-1273f (MIRT783312), hsa-miR-143-3p (MIRT783811), hsa-miR-147a (MIRT783856), hsa-miR-3622a-5p (MIRT784976), hsa-miR-3911 (MIRT785428), hsa-miR-4269 (MIRT785560), hsa-miR-4708-5p (MIRT786482), hsa-miR-4710 (MIRT786499), hsa-miR-4770 (MIRT786696), hsa-miR-4792 (MIRT786788), hsa-miR-574-5p (MIRT787873), hsa-miR-6088 (MIRT788038), hsa-miR-644a (MIRT788184), hsa-miR-6715b-5p (MIRT788450), hsa-miR-6768-5p (MIRT788631), hsa-miR-6867-5p (MIRT789213), hsa-miR-7855-5p (MIRT789601), hsa-miR-8485 (MIRT789865).

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA: miRNA* duplex is loaded into the RISC, the miRNA* is removed and degraded. The strand of the miRNA: miRNA* duplex that is loaded into the RISC is the strand whose 5′ end is less tightly paired. In cases where both ends of the miRNA: miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of a nNOS can be effected using an antisense polynucleotide capable of specifically hybridizing with an mRNA transcript encoding nNOS.

Design of antisense molecules which can be used to efficiently downregulate a nNOS must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7 (2): 236-7; Gait, Cell Mol Life Sci. (2003) 60 (5): 844-53; Martino et al. J Biomed Biotechnol. (2009) 2009:410260; Grijalvo et al. Expert Opin Ther Pat. (2014) 24 (7): 801-19; Falzarano et al, Nucleic Acid Ther. (2014) 24 (1): 87-100; Shilakari et al. Biomed Res Int. (2014) 2014:526391; Prakash et al. Nucleic Acids Res. (2014) 42 (13): 8796-807 and Asseline et al. J Gene Med. (2014) 16 (7-8): 157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65:1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16:1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

For example, suitable antisense oligonucleotides targeted against the nNOS mRNA (which is coding for the nNOS protein) would be of the following sequences:

Korneev et al. [Scientific Reports volume 5, Article number: 11815 (2015)] report a long NAT (Mm-antiNos1 RNA) which is contemplated herein as well as synthetic versions thereof.

Nucleic acid agents can also operate at the DNA level as summarized infra.

Downregulation of nNOS can also be achieved by inactivating the gene (e.g., NOS1) via introducing targeted mutations involving loss-of function alterations (e.g. point mutations, deletions and insertions) in the gene structure.

As used herein, the phrase “loss-of-function alterations” refers to any mutation in the DNA sequence of a gene (e.g., NOS1) which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the e.g. NOS1 may be in a homozygous form or in a heterozygous form. According to this embodiment, homozygosity is a condition where both alleles at the e.g. NOS1 locus are characterized by the same nucleotide sequence. Heterozygosity refers to different conditions of the gene at the e.g. NOS1 locus.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8, 129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example, to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30 (5): 460-5; Miller et al. Nat Biotechnol. (2011) 29:143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

ZFIN commercializes (among other vendors) nNOS specific Talen editing tools (ZDB-TALEN-181114-1).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337:816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

The following vendors provide CRISPR-based products for NOS1 editing:

Applied Biological Materials (abm): CRISPR Clones for NOS1.

NOS1 CRISPR sgRNA+Cas9 as ready-to-use knockout vector or virus:

Cas9 Proteins, Cas9 Nuclease Vector/Virus, Cas9-Expressing Cell Lines also available.

OriGene CRISPR knockouts for NOS1

GA103232

GA202933

Synthego CRISPR Products for NOS1: DIY CRISPR Kits: Gene Knockout Kit, Synthetic sgRNA, Cas9/Engineered Cells: Immortalized KO Pools, KO Clones, iPSC KO, iPSC KI/Free Bioinformatics Tools: CRISPR Knockout Design Tool, CRISPR Analysis Tool VectorBuilder CRISPR vectors for NOS1 (ie. knockout, knockin, CRISPRa, CRISPRi) VectorBuilder Virus packaging for NOS1 CRISPR vectors (ie. lentivirus, AAV, adenovirus)

Santa Cruz Biotechnology (SCBT) CRISPR for NOS1

NOS1 CRISPR/Cas9 KO Plasmid (h)

NOS1 CRISPR Activation Plasmid (h)

NOS1 ZCRISPR plasmid (h) for the human target is available from Santa-Cruz as well as from other vendors

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”-involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy-involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31 (23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the expression level and/or activity of nNOS may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

Modified nucleic acids, including modified DNA or RNA molecules, may be used in the in place of naturally occurring nucleic acids in the polynucleotides described herein. Modified nucleic acids can improve the half-life, stability, specificity, delivery, solubility, and nuclease resistance of the polynucleotides described herein. For example, siRNA agents can be partially or completed composed of nucleotide analogs that confer the beneficial qualities described above. As described in Elmen et al. (Nucleic Acids Res. 33:439-447, 2005), synthetic, RNA-like nucleotide analogs (e.g., locked nucleic acids (LNA)) can be used to construct siRNA molecules that exhibit silencing activity against a target gene product.

Modified nucleic acids include molecules in which one or more of the components of the nucleic acid, namely sugars, bases, and phosphate moieties, are different from that which occurs in nature, preferably different from that which occurs in the human body. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone.

Modifications can be incorporated into any double-stranded RNA (e.g., any RNAi agent (e.g., siRNA, shRNA, dsRNA, or miRNA), RNA-like, DNA, and DNA-like molecules. It may be desirable to modify one or both of the antisense and sense strands of a polynucleotide. As polynucleotides are polymers of subunits or monomers, many of the modifications described below occur at a position which is repeated within a nucleic acid, e.g., a modification of a base, or a phosphate moiety, or the non-linking O of a phosphate moiety. In some cases the modification will occur at all of the subject positions in the nucleic acid but in many, and in fact in most, cases it will not. For example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. For example, a phosphorothioate modification at a non-linking O position may only occur at one or both termini, may only occur in terminal regions, e.g., at a position on a terminal nucleotide or in the last 2, 3, 4, 5, or 10 nucleotides of a strand, or may occur in double strand and single strand regions, particularly at termini. Similarly, a modification may occur on the sense strand, antisense strand, or both. In some cases, the sense and antisense strand will have the same modifications or the same class of modifications, but in other cases the sense and antisense strand will have different modifications, e.g., in some cases it may be desirable to modify only one strand, e.g., the sense strand.

Two prime objectives for the introduction of modifications into the polynucleotides described herein is their increased protection from degradation in biological environments and the improvement of pharmacological properties, e.g., pharmacodynamic properties, which are discussed further below. Other suitable modifications to a sugar, base, or backbone of a polynucleotide are described in PCT Publication No. WO 2004/064737, hereby incorporated by reference. A polynucleotide can include a non-naturally occurring base, such as the bases described in PCT Publication No. WO 2004/094345, hereby incorporated by reference. A polynucleotide can include a non-naturally occurring sugar, such as a non-carbohydrate cyclic carrier molecule. Exemplary features of non-naturally occurring sugars for use in the polynucleotides described herein are described in PCT Publication No. WO 2004/094595, hereby incorporated by reference.

Any of the polynucleotides described herein can include an internucleotide linkage (e.g., the chiral phosphorothioate linkage) useful for increasing nuclease resistance. In addition, or in the alternative, a polynucleotide can include a ribose mimic for increased nuclease resistance. Exemplary internucleotide linkages and ribose mimics for increased nuclease resistance are described in U.S. Patent Application Publication No. 2005/0164235.

Any polynucleotide described herein can include ligand-conjugated monomer subunits and monomers for oligonucleotide synthesis. Exemplary monomers are described in U.S. Patent Application Publication No. 2005/0107325.

Any polynucleotide can have a ZXY structure, such as is described in U.S. Patent Application Publication No. 2005/0164235.

Any polynucleotide can be complexed with an amphipathic moiety. Exemplary amphipathic moieties for use with RNAi agents are described in U.S. Patent Application Publication No. 2005/0164235.

By another possibility the DNA sequence coding for the antisense or iRNA, under a suitable control element (promoter) promotor is delivered to the subject. By one option the expression control element is it is a universal promotor, such as CMV and U1snRNA. According to an embodiment of the invention the expression regulatory element is a neuron specific promoter so that expression takes place only in neurons giving the sequence expresses (antisense) selectivity to neurons. Non-limiting examples of such neuron-specific promotors are: synapsin I promoter CamkII, MeCP2, NSE and Hb9.

Alternatively the sequences can be delivered to the CNS by delivery to the brain (see below) and preferably by use of viral delivery system. A preferred embodiment is by adeno-associated virus (AAV) and in particular by adeno-associated virus of serotype 9 (AAV9) delivery that has become a preferred vector for CNS delivery, due to its increased ability to cross the blood-brain barrier compared to other AAV serotypes. Additional viral delivery systems are Lentivirus and herpes simplex viruses.

### Agents Acting at the Protein Level:

### Antibodies:

According to specific embodiments the agent capable of downregulating nNOS is an antibody or antibody fragment capable of specifically binding nNOS. Preferably, the antibody specifically binds at least one epitope of nNOS. As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three-dimensional structural characteristics, as well as specific charge characteristics.

As nNOS is localized intracellularly, an antibody or antibody fragment capable of specifically binding nNOS is typically an intracellular antibody.

Intracellular antibodies, which may also be called intrabodies, are antibodies that are produced in the cell, and bind an antigen within the same cell.

Importantly, the full-length antibody is not functional in the cytosol, prior to secretion, due to its reducing conditions, which affect protein folding and the intramolecular disulfide bonds that are required to maintain the antibody's conformation and stability. The complementarity-determining regions that endow an antibody with its exceptional target specificity are located in the variable regions of both the heavy and light chains. Therefore, it is possible to use antibody fragments incorporating the specificity-providing regions within a single-chain variable fragment (scFv), which can be further engineered for cytosolic stability, to target intracellular antigens (in this case nNOS). The scFv is a single polypeptide, which is a favorable characteristic for in vivo expression, and it has been studied as a therapeutic for viral infections and cancer, among other diseases.

Furthermore, the variable (V) region domain can be used by itself to form a domain antibody or Dab. These can be engineered from conventional human Igs, or also from those from camelids (camel or llama) and cartilaginous fish (carpet or nurse sharks), whose immune systems were found to have evolved high-affinity V-like domains fused to a conserved framework that is reflective of the constant Fc region found in human Ig. It has been reported that single heavy chain V regions or light chain V regions can be expressed inside cells. These are referred to as intracellular domain antibodies, which do not require intramolecular disulfide bonds for stability, hence representing the smallest format of the antibody that retains target specificity while minimizing size—a crucial factor for intracellular targeting.

According to a specific embodiment, the intrabody comprises a signal for ER targeting to cause degradation of the target protein, antibody-antigen interaction-dependent apoptosis that is used to induce programmed cell death through the activation of caspases, and suicide intrabody technology that causes proteolysis of the target protein.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

nNOS specific antibodies are well known in the art. These can be used to prepare antibody fragments and intrabodies for acting in the brain cells (e.g., neurons).

Examples of such antibodies include, but are not limited to 37-2800 by Thermo Fisher, EP1855Y and EPR 24351-6 by abcam.

These can be modified by length and sequence to act in the cell and/or for human use.

### Aptamers:

Another agent which can be used along with some embodiments of the invention to downregulate nNOS is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24 (4): 381-403). Creative Biolabs provide an aptamer developing service according to SELEX.

Agents which Affect nNOS Stability:

Another contemplated agent which can be used to downregulate any of the above described proteins includes a proteolysis-targeting chimaera (PROTAC). Such agents are heterobifunctional, comprising a ligand which binds to a ubiquitin ligase (such as E3 ubiquitin ligase) and a ligand to one of the above described proteins (nNOS) and optionally a linker connecting the two ligands. Binding of the PROTAC to the target protein leads to the ubiquitination of an exposed lysine on the target protein, followed by ubiquitin proteasome system (UPS)-mediated protein degradation.

Dominant Negative nNOS and Competitive Inhibitors:

It will be appreciated that a non-functional analogue of at least a catalytic or binding portion of nNOS can be also used as an agent which downregulates nNOS because it acts as a homodimer.

According to some embodiment, the agent is an amino acid-based agent also referred to as an amino acid based inhibitor.

According to some embodiment, the agent is PIN or nitric oxide synthase interacting protein (NOSIP).

It has been shown that the N-terminus of nNOS could bind to a protein termed PIN which can inhibit nNOS activity. It is found that PIN destabilizes nNOS dimers and inhibits nNOS activity. Recently, it has been demonstrated that PIN inhibits production of NO and O2, not nNOS dimerization.

Another protein that inhibits NO productions is nitric oxide synthase interacting protein (NOSIP) NOSIP and nNOS co-localize in different areas of the central and peripheral nervous systems. NOSIP negatively affects nNOS activity in a neuroepithelioma cell line stably expressing nNOS. In addition, over-expression of NOSIP in cultured primary neurons limits nNOS trafficking to terminal dendrites and direct nNOS to the soma. These findings suggest that NOSIP regulates NO production in the nervous system by regulating the activity and localization of nNOS. NOSIP upregulation by neuronal activity may prevent NO production in neurons.

Alternatively or additionally, small molecule or peptides can be used which interfere with nNOS protein function (e.g., catalytic or interaction).

Another agent which can be used along with some embodiments of the invention to downregulate nNOS is a molecule which prevents nNOS activation or substrate binding.

According to a specific embodiment, the agent binds to an active site of nNOS and blocks substrate binding. Various amino acids define the active site of nNOS. These include, for example, Phenylalanine 589, Tryptophan 592, Tyrosine 593, Glutamate 597, Aspartate 602, Arginine 601, Methionine 341, Histidine 342, Tyrosine711.

### Small Molecule Inhibitors:

According to a specific embodiment, small molecules that are inhibitors of the neuronal NO synthase can be used as the active agents. A non-limiting example is N^{ω}-Nitroarginine, which is well known to be a potent competitive inhibitor of nNOS that binds to the active site of the enzyme and blocks substrate binding, but it shows little selectivity over eNOS.

Arginine analogs for instance can be used as nNOS competitive inhibitors.

nNOS inhibitors that can be used in accordance with the invention include, but are not limited to, 7-nitro-indazole (7-NI), L-N^{G}-Methyl-L-arginine (L-NMMA), L-N^{G}-Propyl-L-arginine (N-PLA), L-N^{G}-Nitroarginine (L-NNA), L-N^{G}-Nitroarginine methyl ester (L-NAME), L-Thiocitrulline, S-Methyl-L-Thiocitrulline, S-Methyl-L-Thiocitrulline, Ethyl-L-NIO, Vinyl-L-NIO, 7-NI-Br (3-Bromo-7-nitroindazole), and Methylene blue.

Inhibitors available from Tocris, as well as other vendors, include, but are not limited to ARL 17477 dihydrochloride, 3-Bromo-7-nitroindazole, IC 87201 (which is nNOS-PSD95 protein-protein interaction inhibitor), L-NIO dihydrochloride and N^{ω}-Propyl-L-arginine hydrochloride.

Inhibitors available from ApexBio as well as other vendors, include, but are not limited to (S)-Methylisothiourea sulfate, 1400W dihydrochloride, 2,4-Diamino-6-hydroxypyrimidine, 2-Iminopiperidine hydrochloride and 3-Bromo-7-nitroindazole.

According to a specific embodiment, the agent (or selective nNOS inhibitor) is JI-8 (cis-N¹-[4-6-amino-(4-methylpyridin-2-yl methyl) pyrrolidin-3-yl]-N²-[2-(3″-fluorophenyl)ethyl]ethane-1,2-diamine) and structural analogs thereof such as described, for example, in Yu et al., Dev Neurosci. 2011 October; 33 (3-4): 312-319; Ji et al., Ann Neurol. 2009b; 65:209-217; and Ji et al, J Am Chem Soc. 2008; 130:3900-3914, which are incorporated by reference as if fully set forth herein. According to a specific embodiment, the agent is N^{ω}-Nitroarginine.

According to a specific embodiment, the agent reduces a level of an NO precursor.

According to a specific embodiment, the agent increases a level of GSNO reductase level, thereby reducing the level of the NO precursor.

U.S. Patent Application Publication No. 2021/0239400, which is incorporated by reference as if fully set forth herein, discloses nNOS inhibitors which can be used in accordance with the present teachings.

US20200222714 discloses a phototherapy for the inhibition of nNOS activity.

According to a specific embodiment, the nNOS inhibitor (the agent) is 7-NI, including structural analogs thereof.

According to some of any of the embodiments described herein, structural analogs of 7-NI are collectively represented by Formula I:

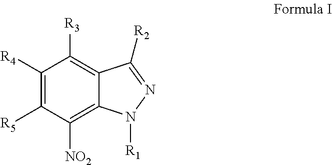

or a pharmaceutically acceptable salt thereof,
wherein:
R₁ is selected from hydrogen, alkyl and cycloalkyl and is preferably hydrogen; and
R₂-R₅ are each independently selected from hydrogen, alkyl, cycloalkyl, aryl, heteroalicyclic, heteroaryl, halo, haloalkyl, hydroxy, alkoxy, aryloxy, thiol, amine, carboxylate, thiocarboxylate, a poly(alkylene glycol moiety), a monosaccharide, a disaccharide, and an oligosaccharide.

According to a specific embodiment, one of R₂-R₅ is halo, including fluoro, chloro, bromo, and iodo, and is preferably bromo. According to a specific embodiment, R2 is halo, preferably bromo. Such a compound is known as Br-7-NI.

According to a specific embodiment, each of R₂-R₅ is hydrogen. Such a compound is known and is referred to herein as 7-nitro-indazole (7-NI).

According to some embodiments, compounds represented by Formula I act as nNOS inhibitors, preferably as selective nNOS inhibitors, as defined herein.

According to some embodiments, compounds represented by Formula I are in a form of pharmaceutically acceptable salts thereof.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation and an acid addition salt thereof, which is also referred to herein as an anionic salt, and/or a deprotonated amine and a base addition salt thereof, which is also referred to herein as a cationic salt.

The base addition salts may include a cation counter-ion such as sodium, potassium, ammonium, calcium, magnesium and the like, that forms a pharmaceutically acceptable salt. Exemplary such cationic salts are described in further detail hereinunder and in the Examples section that follows.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

Exemplary such anionic salts are described in further detail hereinunder and in the Examples section that follows.

### Pharmaceutical Compositions:

The agent (e.g., nNOS inhibitor) as described herein in any of the respective embodiments can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

It will be appreciated that the present teachings contemplate pharmaceutical compositions regardless of their use and each embodiment should be considered individually or in combination with other embodiments.

Herein the term “active ingredient” refers to the agent accountable for the biological effect.

The terms “active ingredient”, “active agent” and “therapeutically active agent” are used herein interchangeably.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

As mentioned, the present teachings also contemplate the use of delivery vehicles dependent on the chemical structure of the active agent (i.e., nNOS inhibitor).

Delivery vehicles. i.e., carriers such as liposomes, nanocapsules, microparticles, microspheres, lipid particles, vesicles, and the like can be used to deliver the nucleic acid molecules described herein.

The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868; and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 ANG, containing an aqueous solution in the core.

Alternatively, nanocapsule or nanoparticle formulations may be used. Nanocapsules can generally entrap substances in a stable and reproducible way. Nanoparticles can be used to transport drugs through the BBB when administered intravenously as well as the factors that influence its transportation.

NPs are colloidal carriers that can have a natural or synthetic origin and can vary from 1 to 1000 nm in size. Synthetic NPs may be prepared from polymeric materials such as poly(ethylenimine) (PEI), poly(alkylcyanoacrylates), poly(amidoamine) dendrimers (PAMAM), poly(ε-caprolactone) (PCL), poly(lactic-co-glycolic acid) (PLGA), polyesters (poly(lactic acid) (PLA), or from inorganic materials such as gold, silicon dioxide (silica), among others. These carriers can transport drugs by adsorbing, entrapping or bounding covalently to them. Natural NPs are produced from natural polymers, such as polysaccharides (chitosan and alginate), amino acids (poly(lysine), poly(aspartic acid) (PASA)), or proteins (gelatin and albumin). Natural NPs have the advantage of providing biological signals to interact with specific receptors/transporters expressed by endothelial cells.

A number of ligands have been conjugated to NPs to facilitate BBB penetration. Such molecules can be grouped into four different types: (i) ligands that mediate the adsorption of proteins from the bloodstream that interact directly with BBB receptors or transporters; (ii) ligands that have direct interaction per se with BBB receptors or transporters; (iii) ligands that increase charge and hydrophobicity; and (iv) ligands that improve blood circulation time (e.g. PEG).

Other methods for assisting the NPs to cross the blood-brain barrier would include but are not limited to receptor mediated transport, transporter mediated transport, absorptive mediated transport, and cell penetrating transport.

Another method includes the use of retrograde tracers based on axonal transport from the periphery such as disclosed in Filler et al. 2010 BMC Neuroscience volume 11, Article number: 8 “Tri-partite complex for axonal transport drug delivery achieves pharmacological effect”. According to an embodiment the tripartite molecular construction concept involves an axonal transport facilitator molecule, a polymer linker, and a retrograde tracer.

As discussed herein and known in the art, axonal transport can be afforded by neuronal tracers, also referred to as histochemical tracers, which are compounds typically used to reveal the location of cells and track neuronal projections. A retrograde tracer is taken up in the terminal or along the axon or other neuronal processes and transported to the cell body, whereas an anterograde tracer moves away from the cell body of the neuron. The term “retrograde tracer” refers to a molecule that is characterized by the capacity to effect axonal transport from the periphery to the DRG. Non-limiting examples of retrograde tracers are provided in, for example, Xiangmin Xu. et al., Neuron, 2020, 107 (6), 1029-1047; Christine, S. et al., Frontiers in Neuroscience, 2019, 13, 897; Kumar, P., Mater Methods 2019; 9:2713; Lanciego, J. L. et al., Brain Structure and Function, 2020, 225, 1193-1224; and Yao, F. et al., PLoS ONE, 13 (10), e0205133, the contents of which is incorporated herein by reference.

Exemplary retrograde tracers include horseradish peroxidase (HRP), dextran, isolectin, isolectin B4 (IB4), cholera toxin subunit B, wheat germ agglutinin (WGA), hydroxystilbamidine (a fluorescent dye), viral based tracers such as RABV, and any known axonal retrograde transport agent or tracer.

In the context of the present invention, the term “retrograde tracer residue” or “a residue of a retrograde tracer” interchangeably refer to the part of the conjugate that confers or allows axonal transport of the conjugate from the locus of administration of the conjugate to the perikaryon.

Mammalian virus vectors that can be used to deliver RNA include oncoretroviral vectors, adenovirus vectors, Herpes simplex virus vectors, and lentiviruses.

In particular, HSV vectors are tropic for the central nervous system (CNS) and can establish lifelong latent infections in neurons.

The AAVs may be delivered to a subject in compositions according to any appropriate methods known in the art. The AAV, preferably suspended in a physiologically compatible carrier (e.g., in a composition), may be administered to a subject, e.g., a human, mouse, rat, cat, dog, sheep, rabbit, horse, cow, goat, pig, guinea pig, hamster, chicken, turkey, or a non-human primate. In certain embodiments, compositions may comprise an AAV alone, or in combination with one or more other viruses (e.g., a second AAV encoding having one or more different transgenes).

Suitable carriers may be readily selected by one of skill in the art in view of the indication for which the AAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the AAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The dose of AAV virions required to achieve a desired effect or “therapeutic effect,” e.g., the units of dose in vector genomes/per kilogram of body weight (vg/kg), will vary based on several factors including, but not limited to: the route of AAV administration, the level of gene or RNA expression required to achieve a therapeutic effect, the specific disease or disorder being treated, and the stability of the gene or RNA product. One of skill in the art can readily determine an AAV virion dose range to treat a subject having a particular disease or disorder based on the aforementioned factors, as well as other factors that are well known in the art. An effective amount of the AAV is generally in the range of from about 10 ul to about 100 ml of solution containing from about 10⁹ to 10¹⁶ genome copies per subject. Other volumes of solution may be used. The volume used will typically depend, among other things, on the size of the subject, the dose of the AAV, and the route of administration. For example, for intrathecal or intracerebral administration a volume in range of 1 ul to 10 ul or 10 ul to 100 ul may be used. For intravenous administration a volume in range of 10 ul to 100 ul, 100 ul to 1 ml, 1 ml to 10 ml, or more may be used. In some cases, a dosage between about 10¹⁰ to 10¹² AAV genome copies per subject is appropriate. In certain embodiments, 10¹² AAV genome copies per subject is effective to target CNS tissues. In some embodiments the AAV is administered at a dose of 10¹⁰, 10¹¹, 10¹², 10¹³, 10¹⁴, or 10¹⁵ genome copies per subject. In some embodiments the AAV is administered at a dose of 10¹⁰, 10¹¹, 10¹², 10¹³, or 10¹⁴ genome copies per kg.

In some embodiments, AAV compositions are formulated to reduce aggregation of AAV particles in the composition, particularly where high AAV concentrations are present (e.g., about 1013 GC/ml or more). Methods for reducing aggregation of AAVs are well known in the art and, include, for example, addition of surfactants, pH adjustment, salt concentration adjustment, etc. (See, e.g., Wright et al. (2005) Molecular Therapy 12:171-178.)

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition). Some variation in dosage will necessarily occur depending on the condition of the host. The person responsible for administration will, in any event, determine the appropriate dose for the individual host.

Sterile injectable solutions are prepared by incorporating the active AAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present invention into suitable host cells. In particular, the vector delivered components may be formulated for delivery either encapsulated in a lipid particle, a liposome, a vesicle, a nanosphere, or a nanoparticle or the like.

In addition to the methods of delivery described above, the following techniques are also contemplated as alternative methods of delivering the compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations, transdermal matrices (U.S. Pat. Nos. 5,770,219 and 5,783,208) and feedback-controlled delivery (U.S. Pat. No. 5,697,899).

To prepare the present pharmaceutical compositions, a conjugate, a vector, a lipid, a nanoparticle, a liposome, an adjuvant or a diluent may be further admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Toxicity and therapeutic efficacy of the therapeutic compositions, administered alone or in combination with another agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the composition or therapeutic can be administered by an invasive route such as by injection. In further embodiments of the invention, the composition, therapeutic, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present invention.

In order to overcome any issue of the pharmacological agents crossing the blood/brain barrier, intrathecal administration is a further preferred form of administration. Intrathecal administration involves injection of the drug into the spinal canal, more specifically the subarachnoid space such that it reaches the cerebrospinal fluid. This method is commonly used for spinal anesthesia, chemotherapy, and pain medication. Intrathecal administration can be performed by lumbar puncture (bolus injection) or by a port-catheter system (bolus or infusion). The catheter is most commonly inserted between the laminae of the lumbar vertebrae and the tip is threaded up the thecal space to the desired level (generally L3-L4). Intrathecal formulations most commonly use water, and saline as excipients but EDTA and lipids have been used as well.

Compositions can be administered with medical devices known in the art. For example, a pharmaceutical composition of the invention can be administered by injection with a hypodermic needle, including, e.g., a prefilled syringe or autoinjector.

The pharmaceutical compositions of the invention may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

Alternately, one may administer the pharmaceutical composition in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the brain. The liposomes will be targeted to and taken up selectively by the desired tissue.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

As used herein, the terms “therapeutically effective amount”, “therapeutically effective dose” and “effective amount” refer to an amount of the present nucleic acid molecules, mutant proteins/polypeptides, and/or modulators that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the agent sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. The present agents/compositions may prevent or delay onset or amelioration of symptoms of the condition in a subject or an attainment of a desired biological outcome, such as correction of neuropathology, e.g., cellular pathology associated with a motor neuronal disease.

For example, the antisense RNA or the iRNA may be delivered as RNA molecules in a suitable carrier that are complementary to a sequence in the nNOS gene, or nNOS mRNA, that can hybridize thereto and reduce its expression. Typically the antisense is chemically modified to increase its stability. In some embodiments, the chemical modification is a modification of a backbone of the oligonucleotide. In some embodiments, the chemical modification is a modification of a sugar of the oligonucleotide. In some embodiments, the chemical modification is a modification of a nucleobase of the oligonucleotide. In some embodiments, the chemical modification increases the intracellular stability of the oligonucleotide. In some embodiments, the chemical modification increases stability of the oligonucleotide in vivo, for example by increasing resistance to proteases. In some embodiments, the chemical modification increases the oligonucleotide's ability to enter cells. In some embodiments the chemical modification improves the ability to bind target RNA. In some embodiments, the chemical modification increases the half-life of the oligonucleotide or improves another relevant pharmokinetic property. In some embodiments, the chemical modification inhibits polymerase extension from the 3′ end of the oligonucleotide. In some embodiments, the chemical modification inhibits recognition of the oligonucleotide by a polymerase. In some embodiments, the chemical modification inhibits double-strand triggered degradation. In some embodiments, the chemical modification inhibits RISC-mediated degradation. In some embodiments, the chemical modification inhibits RISC-mediated degradation or any parallel nucleic acid degradation pathway.

In certain embodiments, the consecutive nucleotide bases are linked by a backbone selected from the group consisting of a phosphate-ribose backbone, a phosphate-deoxyribose backbone, a phosphorothioate-deoxyribosebackbone, a 2′-O-methyl-phosphorothioate a backbone, phosphorodiamidate morpholino backbone, a peptide nucleic acid backbone, a 2-methoxyethyl phosphorothioate backbone, an alternating locked nucleic acid backbone, constrained ethyl backbone, and a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, ligand-conjugated antisense, and a combination thereof.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

### Exemplary Compositions:

The pharmaceutical composition, which is also referred to herein as a “formulation”, is composed of a pharmaceutically acceptable carrier and at least one active agent. The active agent may be a small molecule, an amino acid based compound (peptide, protein, antibody or antibody fragment) or nucleic acid based compound (RNA, including antisense, and iRNA, and DNA), as described herein in any of the respective embodiments.

According to an aspect of some embodiments of the present invention there is provided a pharmaceutical composition comprising a therapeutically active agent which reduces nNOS activity, as described herein in any of the respective embodiments and any combination thereof, and a pharmaceutically acceptable carrier, wherein the composition and the therapeutically active agent are selected such that upon administration of the composition to a subject, reduction in the nNOS activity is effected selectively or preferentially as compared to other NOS isoforms.

According to an aspect of some embodiments of the present invention there is provided a pharmaceutical composition comprising a therapeutically active agent which reduces nNOS activity, as described herein in any of the respective embodiments and any combination thereof, and a pharmaceutically acceptable carrier, wherein the composition and the therapeutically active agent are selected such that upon administration of the composition to a subject, reduction in the nNOS activity is effected selectively or preferentially in neuronal cells or in a nerve tissue of the subject as compared to other tissues.

According to an aspect of some embodiments of the present invention there is provided a pharmaceutical composition comprising a therapeutically active agent which reduces nNOS activity, as described herein in any of the respective embodiments and any combination thereof, and a pharmaceutically acceptable carrier, wherein the composition and the therapeutically active agent are selected such that upon administration of the composition to a subject, reduction in the nNOS activity is effected selectively or preferentially in the peripheral or central nervous system of the subject as compared to other tissues.

According to an aspect of some embodiments of the present invention there is provided a pharmaceutical composition comprising a therapeutically active agent which reduces nNOS activity, as described herein in any of the respective embodiments and any combination thereof, and a pharmaceutically acceptable carrier, wherein the composition and the therapeutically active agent are selected such that upon administration of the composition to a subject, reduction in the nNOS activity is effected selectively or preferentially in the CNS (e.g., the brain) of the subject as compared to other tissues.

As NO is produced in the body and is involved in many physiological processes, it is desired to reduce the amount of NO selectively in a desired tissue (e.g., nerve tissue, the brain, etc.). This can be done for example by using an active agent that is selective for reducing NO in the brain and/or an inhibitor of NO synthase that is specific/selective to neuronal NO synthase. The neuronal NO synthase inhibitor may be a small molecule specific to that neuronal NO-synthase enzyme, an antisense and iRNA specific to the enzyme's mRNA, a DNA capable of expressing these antisense and iRNAs, or a combined system for example CRISPR CAS for selectively reducing expression of neuronal NO synthase. Such agents are described hereinabove.

Alternatively, or in combination, the selectivity of the NO reduction in the nerve tissue (e.g., the brain) may be caused by the properties of the carrier that is delivery selective to the nerve tissue (e.g., CNS), or a delivery system with elements targeted to delivery to the nerve tissue (e.g., CNS) (delivers more to the nerve tissue or CNS than to the body). In the case the selectivity is by the delivery/targeting system the active agent (for example a non-selective NO synthase inhibitor) does not have to be neuronal specific as the delivery (and carrier as will be explained below) are those giving nerve tissue (e.g., CNS) selectivity.

Alternatively, or in combination, the selectivity to the nerve tissue (e.g., CNS, brain) is denoted by the mode of administration (relevant especially for administration of agents that are nucleic acid based) that it administered locally to the desired tissue (e.g., the brain).

Brain selectivity in reduction of NO can be done by one of the following mechanisms:
Decrease in expression of nNOS by using agents selective for the gene/mRNA (that are not active for eNOS and iNOS genes), such as described herein;
Selective inhibitors of nNOS that do not inhibit iNOS and eNOS, such as described herein;
Using reducing agents that are not selective of nNOS but are cable of reducing all NOS [general inhibitors of all NOS (eNOS, iNOS, nNOS)] or agents that decrease the expression of the eNOS (NOS3), iNOS (NOS2), nNOs (NOS1) genes delivered by a delivery system (carriers etc.) that are targeted to deliver their cargo selectively to the CNS, such as described herein; and/or
Using agents that may not be selective to nNOS but are delivered selectively to the brain by intra-brain administration or by brain-targeted delivery vehicle, such as described herein.

According to a specific embodiment, the composition comprises an active agent which is nNOS specific (nNOS selective).

According to a specific embodiment, the administering mode achieves the reduction of nNOS activity in the CNS.

According to a specific embodiment, the composition's administering mode comprises local administration.

Methods of determining activity of nNOS or its expression level/localization are well known in the art and described herein throughout the document. These methods will validate the selectivity of the method to nNOS or to brain-expressed NOS.

According to a specific embodiment, the agent is a selective nNOS inhibitor.

A nNOS inhibitor is considered selective when it reduces nNOS activity to an extent that is higher by at least 20%, or at least 30%, or at least 40%, or at least 50%, compared to other enzymes, particularly other nitric oxide synthase enzymes.

A nNOS inhibitor is considered selective when its dissociation constant (Ki) for nNOS is lower by at least 10-folds, or by at least 100-folds, or by at least 1,000-folds compared to its Ki for the other NOS isoforms.

A nNOS inhibitor is considered selective when its IC50 for nNOS is lower by at least 2-folds, or at least 5-folds, or at least 10-folds, compared to its IC50 for the other NOS isoforms.

According to a specific embodiment, the nNOS reducing composition refers to any pharmaceutical composition, comprising one or more active agents in a carrier, that can reduce the amount of physiologically available NO in the brain. The “neuronal” specificity may be a property of the agent that reduces specifically nNOS and not substantially any other NOS enzymes, as described herein, or the property of the carrier that carriers a general NO reducer to the neurons (either by the delivery system or the mode of administration explained herein).

Without being bound by theory it is suggested that upregulation in nNOS activity/expression (as opposed to iNOS and eNOS) is associated with disease onset or progression. Hence treatment with inhibitors which are not necessarily selective to nNOS would still achieve selective inhibition.

According to some of any of the embodiments described herein, the pharmaceutical composition is such that comprises an active agent as described herein in any of the respective embodiments in an effective amount that reduces an amount of physiologically available nitric oxide in a desired tissue (e.g., a nerve tissue as described herein, for example, the brain), or in neuronal cells, as described herein, preferably in a selective manner, as described herein.

A pharmaceutical composition according to these embodiments can be designed for oral, buccal, sublingual, parenteral, nasal, transdermal, or intratissue administration, particularly for repeated administration or continued, extended or slow release administration, over prolonged periods of time or targeted slow and regulated delivery.

A pharmaceutical composition according to these embodiments can be designed for providing high systemic bioavailability and optionally extended release.

A pharmaceutical composition according to these embodiments can be applied for repeated administration or continued slow release administration, which release therapeutically effective concentrations of the active agent over prolonged periods of time or targeted slow and regulated delivery. The formulations include stable aqueous solutions, liposomes, emulsions and nano and micro-particulate dispersions for subcutaneous or intramuscular injections. Formulations can be in the form of ointments, creams and gels for topical and transdermal delivery of the agent.

As used herein, extended or controlled release refers to retaining active levels of the active agents in the blood or in the designated tissue (e.g., nerve tissue as described herein) for at least 48 hours, from an implant or an injection SC, IM or in tissue.

In an exemplary embodiments, extended or controlled release refers to retaining active levels of the active agents in the blood for at least 2 hours after oral or intranasal administration.

According to an aspect of some embodiments of the present invention there is provided a pharmaceutical composition that comprises an active agent that selectively reduces an activity of nNOS, as described herein in any of the respective embodiments.

According to exemplary embodiments of the present invention, the active agent is JI-8 or a structural analog thereof, as described herein.

According to exemplary embodiments of the present invention, the active agent is 7-NI, or a compound represented by Formula I as described herein in any of the respective embodiments and any combination thereof.

7-NI is water insoluble with poor capabilities of crossing biological membranes. Its target sites are receptors in the nerve system, for example, the brain. Thus, it is required to deliver 7-NI to the desired nerve tissue (e.g., brain, peripheral and/or central nervous system).

According to some embodiments, this is possible using nanoparticles delivered via IV injection or by nose-to-brain nasal spray.

In addition, the formulations described herein can protect 7-NI from early degradation or metabolism while allowing high blood levels over prolonged period of times, depending on the type of delivery system used.

According to some embodiments, a pharmaceutical composition as described herein comprises an active agent as described herein in any of the respective embodiments and any combination thereof, preferably a small molecule agent such as a small molecule nNOS inhibitor (e.g., 7-NI or a compound of Formula I as described herein, or JI-8 or a structural analog thereof, as described herein).

According to some embodiments, the active agent is 7-NI or a compound of Formula I as described herein.

### Pre-Nanodispersion Lipid (PNL) Compositions:

According to some embodiments, the pharmaceutical composition or formulation comprises a carrier that forms a nanodispersion upon contacting an aqueous solution (a pre-nanodispersion carrier). In exemplary embodiments, the composition or formulation is a pre-nanodispersion lipid (PNL) composition or formulation. In exemplary embodiments, the composition comprises an aqueous carrier in which nanoparticles formed of the pre-nanodispersion lipid formulations are dispersed.

Such formulations increase the bioavailability of the active agent (e.g., 7-NI) after oral intake.

A PNL formulation typically includes a carrier that comprises a mixture of at least one lipid, at least one of a surfactant, a water-miscible solvent (e.g., an edible solvent), and optionally further comprise an emulsifier, a dispersing agent and other ingredients. The mixture of surfactants, lipids, and solvent form nanoparticles when contacting an aqueous medium (e.g., a physiological medium) or an aqueous solution.

According to a specific embodiments, the carrier of a PNL formulation comprises components that are considered as GRAS.

Exemplary lipids that are suitable for use in the context of embodiments that relate to PNL formulations include solid and liquid lipids such as, but are not limited to, one or more of: fatty acids, fatty alcohols, triglycerides, hydrogenated vegetable oil, Migliol, vegetable oil and waxes. Specific examples are listed in the Examples section that follows.

Exemplary surfactants that are suitable for use in the context of embodiments that relate to PNL formulations include, but are not limited to, hydrophilic or amphiphilic surfactants or dispersing agent such as, but not limited to, one or more of: surfactants of the Tween® family, surfactants of the Span® family, PEG-lipids and PEG-hydrogenated castor oil. Specific examples are listed in the Examples section that follows.

Exemplary water-immiscible solvents that are suitable for use in the context of embodiments that relate to PNL formulations include, but are not limited to, ethanol, isopropanol, ethyl lactate, ethyl acetate, and propylene glycol. Specific examples are listed in the Examples section that follows.

In some embodiments, the carrier in a PNL formulation is a clear liquid in which the active agent (e.g., 7-NI or a compound of Formula I) is soluble, and which upon contacting an aqueous media, spontaneously disperses into nanoparticles having an average size (e.g., diameter) of, for example, 200 nm or less, preferably below 100 nm, preferable below 50 nm.

The pro-nanodispersion lipid (PNL) based formulations are suitable for oral administration per se, for example, as a liquid formulation loaded in soft gelatin capsule that upon swallowing, the liquid content interacts with the stomach liquids to form a nanodispersion that allow crossing through the GI tract surface into the blood stream.

The PNL formulation serves as solubilizer of the active agent (e.g., 7-NI of a compound of Formula I as described herein) and the nano-dispersion for crossing biomembranes. The smaller the particle size, the bioavailability is improved.

Other forms of pharmaceutical compositions for oral administration, as described herein, are also contemplated for a PNL formulation.

The PNL formulation can be in a liquid form, and can be dispersed in a glass of water or soft drinks and taken as a drink.

The PNL formulation can be comprise an aqueous solution in which the pro-nanodispersion mixture form a nanodispersion (nanoparticles dispersed in the aqueous solution) for injection, oral, nasal administration (e.g., as a nasal spray) or ophthalmic administration (e.g., as eye drops).

The selection of the inactive ingredients (composing the carrier) should consider the safety of the final formulation for the type of delivery and the administration mode.

The PNL composition for injection should include ingredients that are approved for subcutaneous (SC), intramuscular (IM) or intravenous (IV) administration and should not provoke local or systemic toxicity or irritation. Similarly, the composition for eye drops and nasal spray should consider the sensitivity of these tissue surfaces to certain ingredients.

The PNL formulations as described herein can be used for oral, ophthalmic, nasal, transdermal, subcutaneous and buccal delivery, that provides an effective dose to the subject to be treated.

The dispersion in an aqueous medium can be prepared shortly before administration or pre-prepared.

According to some embodiments, the PNL composition or formulation as described herein in any of the respective embodiments comprises as an active agent, 7-NI or a compound of Formula I, or a pharmaceutically acceptable salt thereof, as described herein, for example, a 7-NI lipid salt as described herein.

### Controlled/Extended Release Compositions:

According to some embodiments, the pharmaceutical composition or formulation is configured for controlled or extended release of the active agent.

According to some of these embodiments, such formulations are injectable and implantable formulations that increase the bioavailability of the active agent (e.g., 7-NI or a compound of Formula I or a salt thereof as described herein) at a controlled rate to the surrounding tissue for periods of e.g., days to months.

Injectable controlled and extended release formulations may include: polymeric or liquid based nanoparticles, microparticles, inserts, injectable anhydrous pasty formulation, polymeric liquid formulations that gel into the body to form in-situ implants (e.g., depot) that release the active agent (e.g., 7-NI or a compound of Formula I) for a time period of from 1 day to a few weeks or months). According to some embodiments, such formulations can be prepared from different forms and structures of biodegradable polymers. Exemplary biodegradable polymers include those that have already been used in humans, including, but not limited to, polyesters made from lactide, glycolide, caprolactone and ethylene/propylene carbonate, and sebacic acid based polyanhydride. Protein and polysaccharide based natural polymer carriers are also contemplated, including, but not limited to, gelatin, collagen, hyaluronic acid, oxidized cellulose or amylose, chitosan and the like.

In exemplary embodiments, polymeric implants (e.g. depot) in the form of a wedge or a rod or fiber, are inserted into the body using a trocar or a 16-21G needle. The implant is prepared by either forming a homogeneous melt mix of the polymer and the active agent (e.g., 7-NI or a compound of Formula I as described herein) that is molded into the desired form, suitable for insertion into the body. Alternatively, the devices are prepared by compression molding of a polymer-active agent homogeneous powders.

Polymeric injectable extended release compositions can be configured for, for example, intramuscular or subcutaneous administration. These compositions constantly release the drug over periods from a few days to a few months. In situ depot forming systems for parenteral controlled drug delivery can be in the form of aqueous dispersions of microparticles, polymeric liquids or pastes having a wide range of viscosities. Such systems usually contain a biodegradable carrier dissolved or dispersed in a solvent/co-solvent system, while the drug is either dispersed or dissolved in the liquid phase of the delivery system. Upon Subcutaneous or intramuscular injection, a solid depot is formed at the site of injection. The administration of such a system is far less invasive and costly than the surgical procedures which are often required for implantation.

Exemplary injectable formulations for extended release of the active agents have been designed and exemplified in the Examples section that follows.

According to exemplary embodiments extended release formulations are made of ricinoleic acid and castor oil containing polyesters and polyanhydrides, adapted for use as carriers, such as described, for example, in U.S. Patent Application Publication Nos. 2004/0161464, and 2004/0161464 to Domb). Biodegradable carriers are synthesized from ricinoleic acid oligoesters and aliphatic molecules having at least one carboxylic acid and at least one hydroxy or carboxylic acid group that are liquids or pastes at temperatures below 37° C. In exemplary embodiments, the composition is made of copolymers or ricinoleic acid and sebacic acid at 6:4 to 8:2 w/w ratio that are pasty and injectable at room temperature. These polymers increase their viscosity upon immersion in aqueous medium or injected in tissue.

Injectable microparticles and nanoparticles prepared from PLGA are also contemplated for controlled/extended release. Microspheres and nanospheres can be prepared by dissolving the polymer with the active agent (e.g., 7-NI or a compound of Formula I) in an organic solvent such as chloroform, DMSO, etc., and adding an anti-solvent containing surfactants, such that droplets of the polymer-drug solution are formed whereupon solvent evaporation (in case of water as antisolvent) or diffusion out the droplet to the organic antisolvent, microspheres and nanospheres are formed. The size of the particles can be controlled by the concentration of the polymer-drug solution, the anti-solvent ratio, the surfactant content, the solvent evaporation or leach-out and preparation conditions, including: temperature, solvents used, mixing rate and evaporation aids.

### Particulate Compositions:

According to some embodiments, a pharmaceutical composition as described herein is formulated for injection (e.g., by intravenous administration) and comprise a lipid or polymeric nanoparticles or liposomes in which the active agent (7-NI or a compound of Formula I or a slat thereof) is entrapped or encapsulated.

According to some embodiments, the active agent is incorporated in a biodegradable polymer or lipid carrier.

According to some embodiments, the carrier is in a form of micro- or nano-particles.

According to some embodiments, micro- or nano-particles compositions are formulated for administration via oral administration, oral inhalation, nasal spray, IV, IM or SC injection, or transdermal delivery. According to some embodiments of the invention, the administration is to the brain using nanoparticles delivered to the olfactory tissue.

Exemplary biodegradable polymers suitable for use in the context of these embodiments include, but are not limited to, lactide homo- and copolymers with glycolide or caprolactone, polycaprolactone and its copolymers and other biodegradable polymers in the form of injectable particles dispersed in water, an injectable polymeric pastes or in the form of an insert. Liposomal or a liposphere type of dispersion are also contemplated.

The encapsulated active agent may feature a hydrodynamic surface, such as PEG chains, which reduce RAS ability to eliminate the active agent from the blood stream.

In exemplary embodiments, the active agent is a compound of Formula I in which one or more of R2-R5 is a poly(alkylene glycol) moiety as defined herein.

Methods for making nanoparticles and microparticles include: precipitation of a solution or dispersion of the active agent (e.g., 7-NI or a compound of Formula I or a salt thereof) in the polymer solution and precipitation is an anti-solvent under controlled conditions to form nano and microparticles that entrap the active agent. For example, a solution of PLGA 75:25 dissolved in dichloromethane is mixed with an active agent (e.g., 7-NI or a compound of Formula I or a salt thereof) solution in ethanol to form a clear solution that contain polymer: 7NI at a 100:1 to 65:35 w/w ratio. This solution is added dropwise into stirred heptane containing a surfactant as antisolvent to form particles upon precipitation. Alternatively, the polymer/active agent solution is added to a stirred aqueous solution containing a surfactant such as polyvinyl alcohol or a mixture of Tween® and/or Span® surfactants that upon slow solvent evaporation, nano and microparticles are formed. The size of the particles is controlled and can be tailored by the ratio or solvent: antisolvent, polymer and 7NI concentrations in solution, mixing rate and type of mixing (overhead, stirrer, ultrasound), temperature, etc.

### 7-Nitroindazole Salts Formulations:

Pharmaceutical compositions comprising pharmaceutically acceptable salt of a compound of Formula I (e.g., salts of 7-nitroindazole) are also contemplated. 7-Nitroindazole is a water insoluble molecule that need to be taken for a long time period. Such compositions can be used in repeated administrations or in continued extended (slow release) administration, over prolonged periods of time or targeted slow and regulated delivery. The formulations can increase the bioavailability of 7-Nitroindazole after oral, nasal, short and extended release injectable formulations and transdermal and topical formulations.

The indazole moiety of 7-Nitroindazole is prone to the formation of a salt by eliminating the proton from the N—H of the azole to form a salt with positively charged moieties, including: divalent and trivalent metal ions, ammonium and phosphonium cationic counterparts. In addition, the basic amine that is part of the aromatic ring forms a salt with proton containing molecules, including HCl, phosphate, H₂SO₄ and organic acids. These salts possess properties that are different from the non-salt 7-Nitroindazole, which include: solubility, thermal properties and stability, chemical stability ease of formulation, controlled release, crossing biological membranes and biodistribution.

Exemplary salts and compositions containing same are described in the Examples section that follows.

The salts according to these embodiments can be included in any of the pharmaceutical compositions or formulations as described herein.

### Pharmaceutical Composition for Oral or Nasal Administration:

In some embodiments, for oral or nasal dosage forms, the active agent (e.g., 7-nitroindazole or a compound of Formula I or a salt thereof) can be dissolved or dispersed as nanoparticulate to allow GI absorption. In exemplary embodiments, an anhydrous pre-concentrate solution of the active agent (e.g., 7-nitroindazole or a compound of Formula I or a salt thereof) is formed in a certain mixture of surfactants, lipids and edible solvent, that spontaneously form nanoparticles upon addition to aqueous media, i.e. fluids of the stomach or intestine, that are absorbed in high yield to several fold increase the oral bioavailability of the active agent, as described herein in the context of PNL compositions/formulations. These lipid based formulations can be delivered in soft gelatin capsules or absorbed in porous silica or another carrier and incorporated in common tablet composition and compressed into tablets that upon oral intake, release to the GI liquids to form nanoparticles to be absorb through the GI tract to the blood stream. These lipid based formulations can be delivered nasally, for example, as sprayable formulations.

According to some of these embodiments, a pharmaceutical composition for oral administration such as described herein is formulated in a dosage form that allows at least 25% bioavailability. In exemplary embodiments, an orally-administered composition is used at a dose of from 10 to 1000 mg, or from 200 to 1000 mg, or from 200 to 600 mg, including any intermediate values and subranges therebetween, and even up to 2,000 mg per day, which can be in a once-daily, twice-daily, trice-daily, four-times daily or more unit dosage form. For low doses, 10-100 mg per day, an extended release oral formulations for 1-4 weeks can be designed.

Low dose is also contemplated nasal spray delivery.

For nasal spray, in exemplary embodiments, from 1 to 10 (e.g., 8) spray doses can be applied per day where each dose may contain 1-10 mg, so as to achieve a certain blood level or brain tissue concentration.

In exemplary embodiments, each spray is between 50-200 microliters which may contain 1-10 mg of the active agent. In exemplary embodiments, a daily dose of up to 100 mg is contemplated.

### Pharmaceutical Compositions for Injection:

Injectable polymeric delivery systems are preferably configured to deliver drugs for periods of weeks to month, depending on the daily dose and the formulation. Exemplar injectable formulations are based on poly(lactide-glycolide) copolymers that have been used in the clinic for the extended delivery of drugs such as Risperidone and LHRH analogs.

Additional exemplary formulations for injection, which provide extended release of the active agent include the extended/controlled release compositions or the particulate compositions/formulations as described herein.

The dose for extended release injectable compositions is typically a function of the daily dose multiplied by the number of days the delivery system intends to deliver the active agent. A maintenance dose of the active agent, which can be less than a desirable daily dose is also contemplated in the context of these embodiments. This maintenance dose can be combined with an oral dose or nasal dose or transdermal patch, e.g., at a low daily dose as described herein.

### Pharmaceutical Compositions for Buccal Administration:

According to some embodiments, buccal delivery of the active agent (e.g., 7-nitroindazole or a compound of Formula I or a salt thereof) can be performed using a PNL formulations as described herein in any of the respective embodiments, absorbed in or on a carrier suitable for buccal delivery, for example, in cross-linked polyacrylic acid, Carbopol 940 or Carbopol 970, or/and in hydroxypropyl cellulose (HPC) or hydroxypropyl methyl cellulose (HPMC).

In exemplary embodiments, the active agent (e.g., 7-nitroindazole or a compound of Formula I or a salt thereof) and PNL absorbed solids are mixed at a 10:0 to 6:4 Carbopol to HPC w/w ratio and compressed into tablets of 13 mm in diameter. The more Carbopol in the formulation, the stronger is the adhesion to the buccal.

Other ingredients commonly used in buccal tablets such as flavorings, different polymer compositions and colorants can be added to the powder mix before compression into mucoadhesive tablets. In an exemplary formulation, 7-NI (10 mg) is dissolved in 200 microliters of a clear solution of Tween®20, Span®80, sesame oil and Chremophor 40R at 1:1:0.5:0.2 w/w. This solution is absorbed in 400 mg of Carbopol 940 and mixed with 100 mg HPC. The dry powder is compressed into tablet of 13 mm using pressure of 2 tones. The tablet adheres well to the human buccal and remain in place for 4 hours while eroding and eliminating and releasing 7 IN to the buccal with time.

Buccal delivery is typically dose limited by the penetration through the buccal and in some embodiments, administration is of small doses of 1-100 mg per day.

### Pharmaceutical Compositions for Transdermal Administration:

Transdermal formulations are usually applied for low dose delivery of an active agent since the skin penetration is low for most molecules, including 7-NI. When a maintenance dose for an adult or child from a large surface patch is suitable, such a patch can be prepared by embedding the active agent (e.g., 7-nitroindazole or a compound of Formula I or a salt thereof) in a pasty formulation that is applied onto a common transdermal patch. To enhance active agent penetration through the skin, enhancers such as azone, PEG and other agents may be added.

For transdermal delivery, attention should be given to the local irritation of the composition. Tolerable doses and formulations are used with the objective to reach the highest dose possible so that the skin surface area to be applied for the delivery of the active agent is adjusted to the desired dose per hour.

In some embodiments, a transdermal patch can be applied to the skin for one week, and, if desired, repeatedly replaced every week. Alternatively, a transdermal patch is applied to the skin of the subject on daily basis, or every 2, 3 or 4 days.

Delivery of a nNOS Reducing Agents—by a Brain-Selective Delivery System

According to some embodiments of the present invention there is provide a pharmaceutical composition that comprises a nucleic acid-based nNOS inhibitor (e.g., (a promotor and/or a gene and/or other nucleic acid-based materials) as described herein in any of the respective embodiments.

The delivery of such nNOS inhibitors can be done by any delivery system suitable for delivery to the CNS, either by direct delivery or by systemic delivery.

A non-limiting delivery system or delivery vehicle of such nNOS inhibitors to the CNS are nanoparticles, typically having a size of less than 200 nm. These may include lipid-based nanoparticles, polymer nanoparticles, dendrimers and inorganic nanoparticles, some of which may be tailored to pass through the BBB.

Another manner of administration is by the use of liposomes that can also pass the BBB.

Preferably, it is possible to use actively targeted delivery by using ligands of transporters or receptors to enhance nanoparticle uptake across the BBB. The preferred pathway for this approach is receptor (or transporter)-mediated transcytosis by which a cargo (e.g., nanoparticles) transports between the apical and basolateral surface in the brain ECs. For example, low-density lipoproteins undergo transcytosis through the ECs by a receptor-mediated process, bypassing the lysosomal compartment and releasing at the basolateral surface of the brain side.

Another vehicle for brain delivery includes exosomes which are small extracellular vesicles secreted by cells. The major advantage of exosomes versus other synthetic nanoparticles is their non-immunogenic nature, leading to a long and stable circulation.

As the BBB contains transporters to amino acids, using a BBB naturally present arginine transporter for the delivery may be a simple viable approach for delivery to the brain.

Another approach is to use compounds or electric stimulation to transiently open the BBB and allow high concentrations of systemically administered sequences to reach the brain. An example of such a compound is cereport (a bradykinin analog) or regadenoson (an adenosine receptor agonist).

Another manner to increase penetration is via Ultrasound has become an attractive technique to facilitate drugs to cross the BBB in recent years. Microbubble-enhanced diagnostic ultrasound (MEUS), a non-invasive technique, effectively helped drugs cross the BBTB. Another approach is transcranial magnetic stimulation (TMS), which stimulates neuronal activity and increases glutamate release, facilitated drug delivery across the BBB. (Review by Xiaowei Dong, Theranostics. 2018; 8 (6): 1481-149-incorporated in it's entirely herein by reference).

Routes of administration of the desired delivery vehicles may be systemic administration without further radiation based manipulations (e.g., using particles, that inherently enter the BBB); systemic administration using compounds in conjunction with various manipulations (e.g., in conjunction with microcrobubble-enhanced diagnostic ultrasound (MEUS), transcranial magnetic stimulation (TMS)) to transiently open the BBB; nasal administration.

Vehicles for a targeted delivery to the CNS (e.g., the brain) can also be utilized as carrier in pharmaceutical compositions that comprise any of the other agents that reduce nNOS activity as described herein in any of the respective embodiments and any combination thereof.

Delivery of Agents that Reduce nNOS Activity by a Direct Administration to the CNS (e.g., the Brain)

By one embodiment the administration is by direct injection into the parenchyma or by injection into the cerebrospinal fluid via the intracerebroventricular or intrathecal (cisternal or lumbar) route, as described herein in any of the respective embodiments and any combination thereof.

Preferred local administration to the brain can be carried out by administration to the cerebrospinal fluid via intracerebroventricular route. Another option is delivery to the cisterna magna route of injection has been recently adopted as an alternative method for delivery into cerebrospinal fluid (CSF) which results in wide-spread gene delivery throughout the CNS.

### Exemplary Uses:

According to an aspect of some embodiments of the present invention there is provided a pharmaceutical composition as described herein in any of the respective embodiments and any combination therein, which comprises as an agent that reduced nNOS activity a compound of Formula I or a salt thereof, as described herein in any of the respective embodiments.

According to some embodiments, the pharmaceutical composition is for use in the treatment of any of the diseases or conditions as described herein. Examples include, but are not limited to, ASD, as defined herein, Alzheimer's disease, Parkinson's disease, Huntington's disease, Multiple sclerosis, Schizophrenia, addiction, ALS, Epilepsy, bipolar disease, migraine and in addition all kinds of neurodevelopmental disorders as well as neurodegenerative diseases, such as described in Tripathi et al. Redox Biol. 2020:101567.

According to an aspect of some embodiments of the present invention there is provided a method of treating an autism spectrum disorder, as defined herein in any of the respective embodiments and any combination thereof, in a subject in need thereof, the method comprising administering to the subject an effective amount of a composition comprising an active agent which reduces nNOS activity, as described herein in any of the respective embodiments and any combination thereof.

According to some embodiments, reduction in the nNOS activity is selectively or preferentially in neuronal cells or a nerve tissue of the subject (e.g., the CNS or the brain) as compared to other tissues, as described herein.

According to some embodiments, the reduction in the nNOS activity is in a nerve tissue (CNS, e.g., brain) or in peripheral organs which comprise neuronal cells that express nNOS, as described herein.

According to some embodiments, reduction in the nNOS activity is selective compared to other NOS isoforms, as described herein.

According to some embodiments, reduction in the nNOS activity is selective such that an amount of physiologically available NO is reduced selectively in neuronal cells of the subject, as described herein.

According to some embodiments, reduction in the nNOS activity is selective such that an amount of physiologically available NO is reduced selectively in the CNS or the brain of the subject, as described herein.

According to an aspect of some embodiments of the present invention there is provided a method of treating an autism spectrum disorder, as defined herein in any of the respective embodiments and any combination thereof, in a subject in need thereof, the method comprising administering to the subject an effective amount of a pharmaceutical composition as described herein in any of the respective embodiments and any combination thereof, including the exemplary compositions as described herein.

According to an aspect of some embodiments of the present invention there if provided a pharmaceutical composition as described herein in any of the respective embodiments and any combination thereof, including the exemplary compositions as described herein for use in treating an autism spectrum disorder, as described herein in any of the respective embodiments, in a subject in need thereof.

According to an aspect of some embodiments of the present invention there is provided a method of treating a neural-derived cancer, as defined herein in any of the respective embodiments and any combination thereof, in a subject in need thereof, the method comprising administering to the subject an effective amount of a composition comprising an active agent which reduces nNOS activity, as described herein in any of the respective embodiments and any combination thereof.

According to some embodiments, reduction in the nNOS activity is selectively or preferentially in a respective nerve tissue or respective neuronal cells of the subject as compared to other tissues, as described herein.

According to some embodiments, reduction in the nNOS activity is selective compared to other NOS isoforms, as described herein.

According to some embodiments, reduction in the nNOS activity is selective such that an amount of physiologically available NO is reduced selectively in the respective nerve tissue or respective neuronal cells, as described herein.

According to an aspect of some embodiments of the present invention there is provided a method of treating a neural-derived cancer, as defined herein in any of the respective embodiments and any combination thereof, in a subject in need thereof, the method comprising administering to the subject an effective amount of a pharmaceutical composition as described herein in any of the respective embodiments and any combination thereof, including the exemplary compositions as described herein.

According to an aspect of some embodiments of the present invention there is provided a pharmaceutical composition as described herein in any of the respective embodiments and any combination thereof, including the exemplary compositions as described herein for use in treating a neural-derived, as described herein in any of the respective embodiments, in a subject in need thereof.

According to some of any of the embodiments described herein, the neural-derived cancer is neuroblastoma, as defined herein.

According to some of any of these embodiments, the pharmaceutical composition is an extended release composition as described herein in any of the respective embodiments and any combination thereof.

According to some of these embodiments, the methods and uses as described herein are effected by repetitively administering the composition (e.g., by injection, or transdermally, as described herein) in a time interval between administrations which is at least one day, preferably at least 2 days, at least 3 days, at least 4 days, at least one week, optionally, at least 2 weeks, at least one month, at least two months, and more. According to some of these embodiments, this can be effected using a controlled-extended release composition as described herein in any of the respective embodiments.

According to some of any of the embodiments described herein the subject is a mammal, for example, a human being.

According to some of any of the embodiments described herein the subject is afflicted by a disease or condition as described herein in any of the respective embodiments, for example, a chronic disease or condition such as ASD or neuroblastoma.

According to some of any of the embodiments described herein, the subject is a neonatal or an infant, for example, is aged from 0 to 10 years.

According to some of any of the embodiments described herein, the subject is an adolescent, for example, is aged between 10 to 20 10 years.

According to some of any of the embodiments described herein, the subject is an adult, for example, is aged at least 20 years.

### Animal Model:

The present invention is based on a second finding that administration of S-nitroso-N-acetylpenicillamines (SNAP), is an NO donor that increases the NO availability induced ASD-like phenotypes in the C57BL/6 mouse model.

As research of ASD is difficult by lack of good animal models this finding paves the way of creating an ASD model in a laboratory animal by administering to a WT animal an effective amount of an NO donor.

Thus, according to an aspect of the invention there is provided a method of producing a laboratory animal model for autism spectrum disorder (ASD), the method comprising administering to said laboratory animal an effective amount of a composition which increases NO levels in a brain of said animal to produce an ASD-like phenotype.

As used herein “an ASD-like phenotype” refers to molecular and/or behavioral phenotype. Examples of molecular markers include, but are not limited to: SYNAPTOPHYSIN, GAD1, PSD-95 and VGAT.

Examples of behavioral parameters and methods of testing them include, but are not limited to, motor activity (open field test, which also examines the anxiety-like behavior), social behavior (three-chambered social test), anxiety-like behavior (elevated plus maze), explorative activity, and repetitive and restricted behavior (novel object recognition), and/or restricted behavior (marble burying test).

According to a specific embodiment, the animal model is a mammal.

According to a specific embodiment, the animal is a rodent, e.g., mouse, rat or rabbit.

According to a specific embodiment, the mouse if a juvenile mouse (e.g., 6 weeks old).

As used herein “a composition which increases NO levels in a brain of said animal” refers to any substance or physical condition which increases nNOS activity, prevents degradation thereof, prevents depletion of NO from the neural tissue above a level that is normal in non-ASD animals.

According to a specific embodiment, the composition comprises a nucleic acid sequence for increasing expression of heterologous (exogenous) nNOS or homolog thereof in the brain. Alternatively or additionally the nucleic acid agent is for increasing an activity of a regulatory region so as to increase expression of endogenous nNOS.

Administration of the NO donor may be systemic or directly to the brain as described herein.

According to a specific embodiment, the administering is systemic.

According to a specific embodiment, the administering is directly into said brain of said animal.

According to a specific embodiment, the composition comprises an NO donor.

According to a specific embodiment, the NO donor comprises S-nitroso-N-acetylpenicillamines (SNAP).

Other exemplary NO donors include, but are not limited to, SNAP, nitrates, nitrites, N-nitroso, C-nitroso, S-nitroso, heterocyclics, metal/NO complexes, diazeniumdiolates; organic nitrates such as nitroglycerin, isosorbide-5-mononitrate, nicorandil, pentaerythritol tetranitrate; sodium nitroprusside (SNP), S-nitrosothiols such as S-nitroso-N-acetylpenicillamine (SNAP) and S-nitrosoglutathione; sydnonimines (e.g., molsidomine, SIN-1); and NONOates (e.g., JS-K, Spermine NONOate, and Proli-NONOate).

The examples section which follows describes the qualification of such an animal model. Also contemplated herein are tissues and cells (as well as cell lines) that can be obtained from such an animal.

Embodiments of the present teachings relate to the use of such animals and cells/tissues derived therefrom in research such as in drug screening and development of diagnostic assays.

According to a specific embodiment there is provided an animal model produced according to the method as described herein.

It is expected that during the life of a patent maturing from this application many relevant agents that reduce nNOS activity as described herein will be developed and the scope of the term “active agent” is intended to include all such new technologies a priori.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

Any of the active agents as described herein (e.g., small molecule active agents) can be in form of a pharmaceutically acceptable salt, as defined herein, a prodrug, a hydrate or a solvate thereof.

As used herein, the term “prodrug” refers to a compound which is converted in the body to an active compound. A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The active agents as described herein can be used as polymorphs and the present embodiments further encompass any isomorph of the active agent and any combination thereof.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an(S) configuration and any combination, and compounds according to some embodiments of the present invention, can have any their chiral centers exhibit an (R) or an(S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

Herein, the terms “amine” and “amino” each refer to either a —NR′R″ group or a —N⁺R′R″R′″ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is not substituted by oxo (unless explicitly indicated otherwise), such that R′, R″ and R′″ are not (for example) carbonyl, C-carboxy or amide, as these groups are defined herein.

An “azide” group refers to a —N═N⁺═N group.

An “alkoxy” group refers to any of an —O-alkyl, —O-alkenyl, —O-alkynyl, —O-cycloalkyl, and —O-heteroalicyclic group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic group, as defined herein.

A “thioaryloxy” group refers to both an —S-aryl and an —S-heteroaryl group, as defined herein.

A “carbonyl” or “acyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxylic acid” group refers to a —C(═O)OH group.

An “oxo” group refers to a ═O group.

An “imine” group refers to a ═N—R′ group, where R′ is as defined herein.

An “oxime” group refers to a ═N—OH group.

A “hydrazone” group refers to a ═N—NR′R″ group, where each of R′ and R″ is as defined herein.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂-R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R′S(═O)₂—NR″— group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″—group, where each of R′ and R″ is as defined herein.

An “O-thiocarbamyl” group refers to an —OC(═S)—NR′R″ group, where each of R′ and R″ is as defined herein.

An “N-thiocarbamyl” group refers to an R′OC(═S) NR″— group, where each of R′ and R″ is as defined herein.

An “S-thiocarbamyl” group refers to an —SC(═O)—NR′R″ group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″—group, where each of R′ and R″ is as defined herein.

A “urea group” refers to an —N(R′)—C(═O)—NR″R′″ group, where each of R′, R″ and R″ is as defined herein.

A “thiourea group” refers to a —N(R′)—C(═S)—NR″R′″ group, where each of R′, R″ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′″ are as defined herein.

As used herein, the term “thiohydrazide” describes a —C(═S)—NR′—NR″R′″ group, where R′, R″ and R′″ are as defined herein.

A “guanidinyl” group refers to an -RaNC(═NRd)-NRbRc group, where each of Ra, Rb, Rc and Rd can be as defined herein for R′ and R″.

A “guanyl” or “guanine” group refers to an RaRbNC(═NRd)- group, where Ra, Rb and Rd are as defined herein.

As used herein, the term “alkylene glycol” describes a —O—[(CR′R″)_{z}-O]_{y}—R′″ end group or a —O—[(CR′R″)_{z}-O]_{y}-linking group, with R′, R″ and R′″ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is referred to herein as oligo (alkylene glycol).

When y is greater than 4, the alkylene glycol is referred to herein as poly(alkylene glycol). In some embodiments of the present invention, a poly(alkylene glycol) group or moiety can have from 10 to 200 repeating alkylene glycol units, such that z is 10 to 200, preferably 10-100, more preferably 10-50.

The term “saccharide” as used herein encompasses monosaccharides, disaccharides and oligosaccharides. The term “monosaccharide”, as used herein and is well known in the art, refers to a simple form of a sugar that consists of a single saccharide molecule which cannot be further decomposed by hydrolysis. Most common examples of monosaccharides include glucose (dextrose), fructose, galactose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms. Monosaccharides are the building blocks of disaccharides and oligosaccharides like sucrose (common sugar).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Part I

### Alleviation of ASD-Like Phenotypes by nNOS Inhibition

### Materials and Methods

Animal models:

SHANK3 and CNTNAP2 mutant mice were obtained from Jackson Laboratory and used for studying behavioral and synaptic abnormalities associated with autism spectrum disorder. Unless otherwise indicated, WT and mutant mice are 6-weeks old.

It was found that SHANK3 mutation leads to an increase of in nitric oxide (NO) levels and to changes in the S-nitroso-proteome in SHANK3 mice. The present inventors have showed that this mutation leads to S-nitrosylation of key proteins involved in vesicle release and synaptic function which could result in autistic-like behaviors (Amal et al. Molecular Psychiatry 2018, www(dot)doi(dot)org/10(dot)1038/s41380-018-0113-6).

### SNAP Treatment Protocol:

S-nitroso-N-acetylpenicillamines (SNAP) was obtained from Tzamal D-Chem. WT or mutated Mice were treated with SNAP intraperitoneally. Each mouse was given the dose of 20 mg/kg body weight on each day of the experiment. Behavioral testing started after 2 hours of treatment. SNAP was first solubilized in DMSO (solubility is 20 mg/ml) and further volume was made up with 0.9% saline. Control animals were treated with vehicle (0.9% saline and DMSO). All animals were sacrificed after 24 hours of last treatment [1].

### 7-NI Treatment:

7-Nitro-indazole (7-NI) was obtained from Tzamal D-Chem.

WT, SHANK3-mutant and/or CASPR2-KO Mice were treated with 7-NI intraperitoneally. Each mouse was given a dose of 80 mg/kg body weight on each day of the experiment, unless otherwise indicated. Behavioral testing started after 2 hours of treatment. 7-NI was first solubilized in DMSO (solubility is 32 mg/ml) and further volume was made up with corn oil, unless otherwise indicated. Control animals were treated with a respective vehicle (e.g., corn oil and DMSO). All animals were sacrificed after 24 hours of last treatment.

### Behavioral Analyses:

Open field test: Mice were tested in an open field (45×45 cm) virtually divided into central and peripheral regions. Animal activity was recorded by video tracking (Noldus Ethovision). Each mouse was allowed to explore the apparatus for 5 minutes. The distance traveled, the number of rears and revolutions, the number of grooming bouts and cumulative grooming time, the number of head shaking or twitches, the number of entries in the center, and the time spent in the central and peripheral regions were recorded. Measures were recorded in 5-minute intervals.

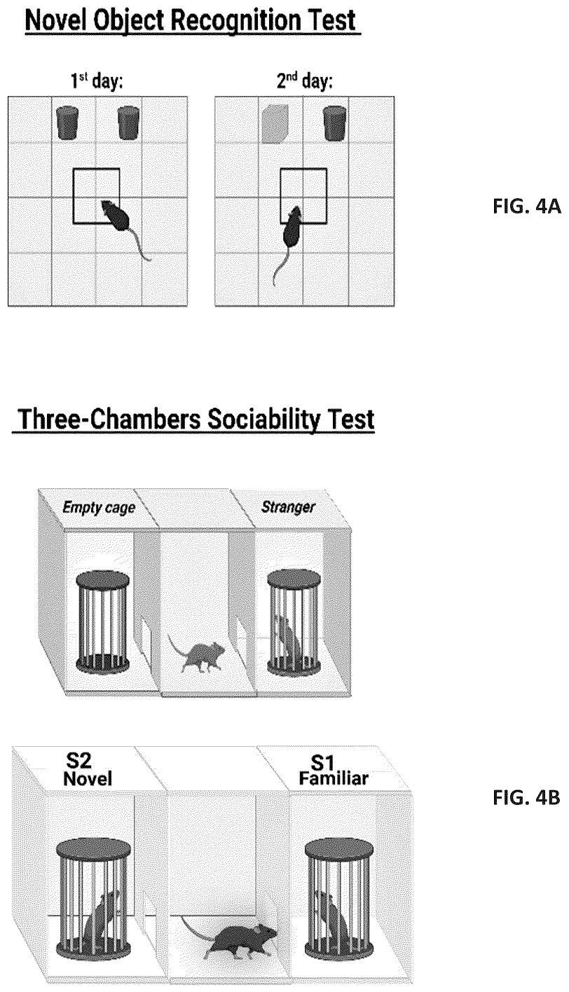

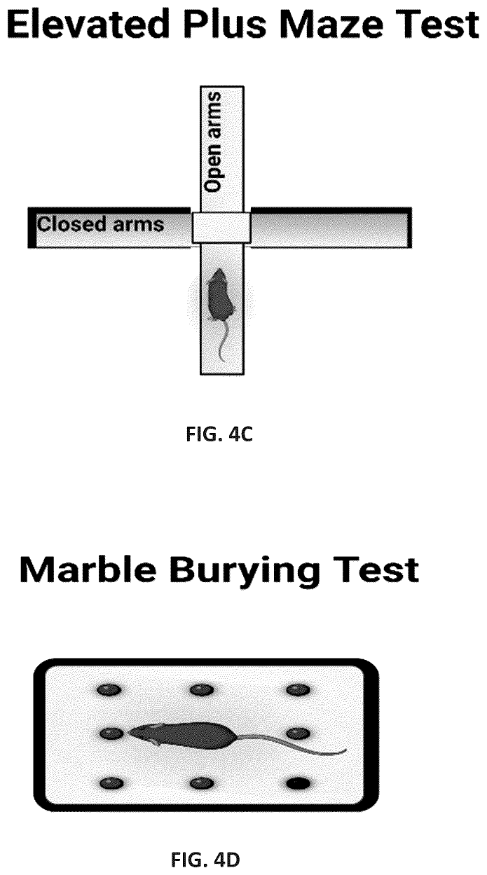

Novel object recognition (see, FIG. 4A): The novel object test for object recognition and memory takes place in a white open field arena (45×45 cm). The test involves a set of two unique novel objects, each about the size of a mouse, constructed from two different materials and non-uniform in shape [4]. The test consisted of one 10-minute habituation session, a 5-minute familiarization session and a 5-minute recognition test, each video tracked (Noldus Ethovision). During the habituation, animals were allowed to freely explore an empty open field. At the end of the session, they were shifted from the open field to a place in a temporary clean holding cage for about 2 minutes. Two identical objects were placed on the median line at 10 cm from each wall and the animal was returned to the open field and allowed to explore the objects for 5 minutes before being returned to its home cage. Next day, one familiar object and one novel object were placed in the open field to the location where the identical objects were placed during the familiarization session and the mouse was allowed to explore them for a 5-minute recognition test. The side of the novel object position was randomly assigned so half of the animals were exposed to a novel object placed on the right of the open field and half of the animals were exposed to a novel object placed on the left of the open field. Between each session, the open field and the objects were carefully cleaned with 70% ethanol and let dry. Familiarization and recognition sessions were scored for total time spent investigating each object, the number of object interactions and the latency of the first object interaction. Time spent in each side during habituation and familiarization and time spent sniffing two identical objects during the familiarization phase were used to examine an innate side bias. Total time spent sniffing both objects was used as a measure of general exploration.

Three-chambered sociability test (see, FIG. 4B): Sociability and preference for social novelty and social recognition were tested in a three-chambered apparatus. The subjected mouse was first placed in the central, neutral chamber and allowed to explore for 10 minutes with all the doors closed. Next, doors were opened and the mouse was allowed to freely explore the other two empty chambers (1 and 2) for an additional 10 minutes. Lack of side preference was confirmed during this habituation.

Two cylindrical wire cages were placed, one in chamber 1 and the other in chamber 2. For the sociability test, the test animal was introduced to the middle chamber and allowed to adjust for 5 minutes. Then, an unfamiliar mouse was introduced into a wire cage in one of the side-chambers, and the other side-chamber remained empty. The time spent by the test mouse exploring the wire cage with an unfamiliar mouse inside was recorded for 5 minutes.

Next, to examine social memory, novel mice is introduced to the empty cage and the tested mice is examined whether they are interested in the novel mice compared to the familiar.

Elevated plus maze test (see, FIG. 4C): The elevated plus-maze consisted of four arms (30×5 cm), two open, and the other two closed. The platform was made of white plexiglass. The apparatus was elevated 45 cm above the floor. The test, described in, was initiated by placing the mouse on the central platform of the maze, facing one of the open arms, and letting it move freely. Each session lasted 5 min. The time spent in the close and open arms was recorded.

Marble Burying test (see, FIG. 4D): The marble-burying assay is a tool for assessing either anxiety-like and/or repetitive-like behaviors in mice. Subjects were tested in a regular clean cage (28 cm long×18 cm wide×12 cm high) with 3 cm of fresh bedding. The subject was first placed in the empty cage for a 5-min habituation. It was then temporary placed in an empty clean cage while 20 dark blue glass marbles (15 mm in diameter) were positioned over the bedding equidistant in a 4×5 arrangement to cover the whole cage surface. The subject was then returned in the test cage and allowed to explore and bury the marbles during a 15-min session that was videotaped. At the end of the session the subject was removed and the number of marbles buried (50% marble covered by bedding material) was recorded.

### Example 1

### Increase of 3-nitrotyrosine (Ntyr) in the ASD Models

3-Nitrotyrosine (3-Ntyr), is a post-translational modification in proteins occurring through the action of a nitrating agent resulting in the addition of a NO₂ group (at the ortho position to the phenolic hydroxyl group), leading to protein tyrosine nitration (PTN). An essential feature of PTN is that it is a stable post-translational modification and does not occur at random. 3-Ntyr represents a reliable marker of nitrosative stress.

10 participants with ASD (age: 2-6, males) and 5 unrelated, age- and gender-matched, control participants, attending regular education and without any neuropsychiatric diagnosis (age: 2-6 males), were recruited from the outpatient neuropediatric clinics in Shaare Zedek Medical Center as part of an ongoing clinical trial (0501-20-SZMC) that assesses biomarkers in children with ASD. The study was approved by the Institutional Review Board at Shaare Zedek Medical Center and participants' parents provided written informed consent prior to participant enrollment.

Western blot was done to determine NTyr levels.

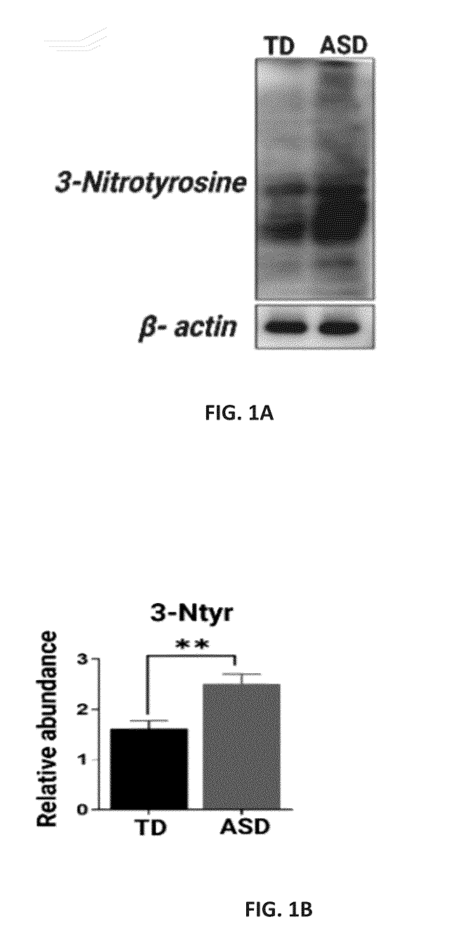

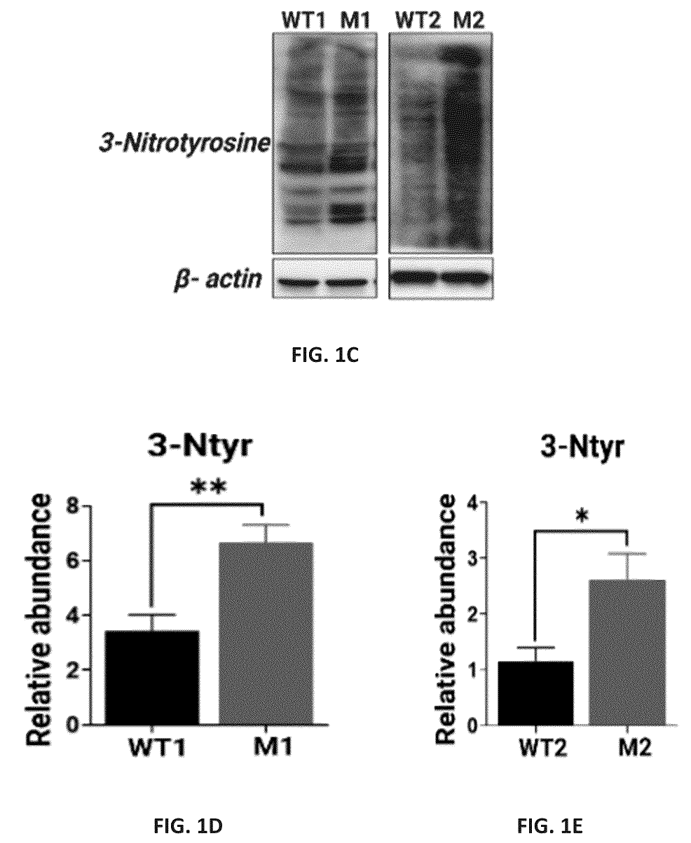

The results are presented in FIGS. 1A-E.

Western blot (WB) analysis of the human blood samples showed a significantly elevated level of Ntyr in ASD compared to their typically developed counterparts (TD), indicating an increased level of nitrosative stress in ASD individuals (FIG. 1A), as is also demonstrated in the qualitative analysis presented in FIG. 1B. **P<0.01. Control (n=5) and ASD (n=10).

WB analysis of cortex tissues prepared from wild-type mice (C57BL/6 mice) and two ASD mouse models-SHANK3 mouse model (M1) and CNTNAP2 mouse model (M2) showed higher levels of Ntyr in both ASD mouse models compared to the wild-type (FIG. 1C), as is also demonstrated in the respective qualitative analyses presented in FIGS. 1D and 1E. *P<0.05. WT (n=5) and M1, M2(n=5).

### Example 2

### SNAP Administration Induces ASD-Like Phenotypes in a C57BL/6 Mouse Model

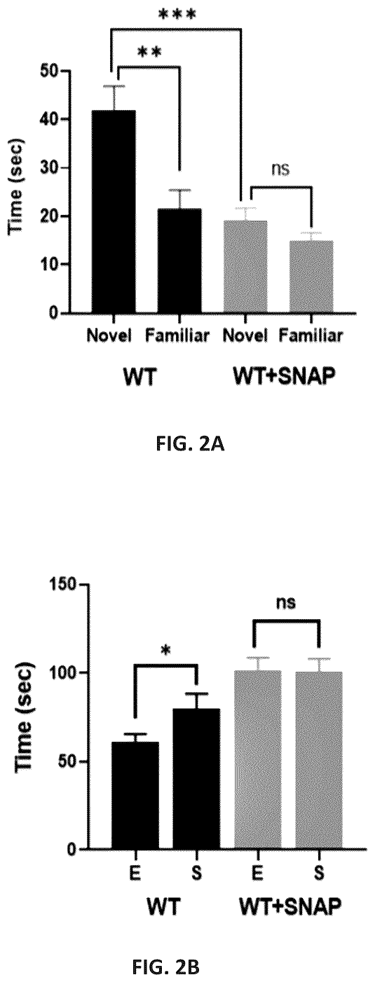

S-nitroso-N-acetylpenicillamines (SNAP), is an NO donor that increases the NO availability, subsequently leading to nitrosative stress and aberrant SNO signaling. Wild-type mice were treated with SNAP intraparentaly (IP), 20 mg/kg/day for 7 days. To examine the potential effect of SNAP administration on mice behaviors, a novel object recognition test (NOR) and sociability test were performed (See, FIGS. 4A-B), and the obtained data are presented in FIGS. 2A-B, respectively.

As shown in FIG. 2A, the NOR test showed that SNAP-treated mice failed to display a preference for the novel object, whereas the control group spent significantly more time exploring the novel object.

As shown in FIG. 2B, the sociability test showed that the control group spent more time interacting with the stranger cage, indicating high sociability. Conversely, the SNAP-treated mice did not exhibit any preference to engage in social interaction.

### Example 3

### 7-NI Administration Reverses the ASD-Like Phenotypes in CNTNAP2 and SHANK3 Mouse Models

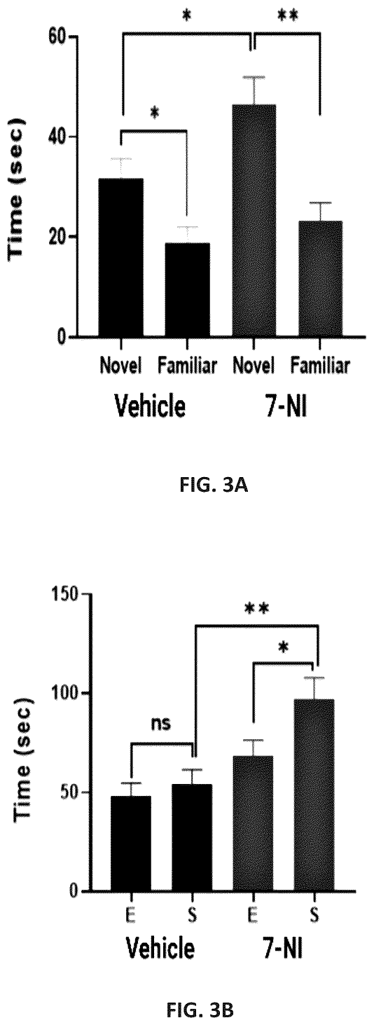

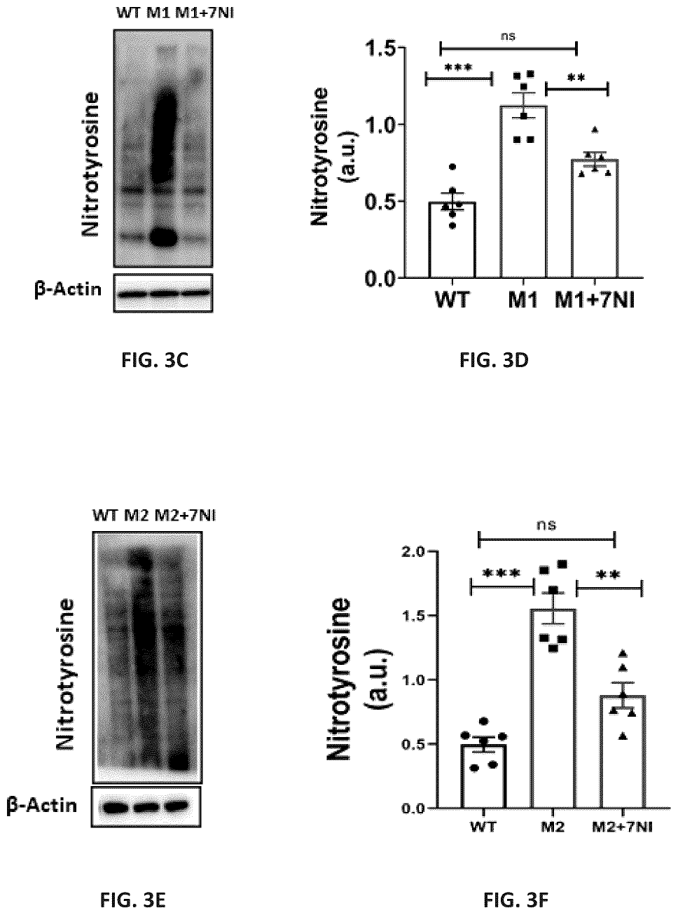

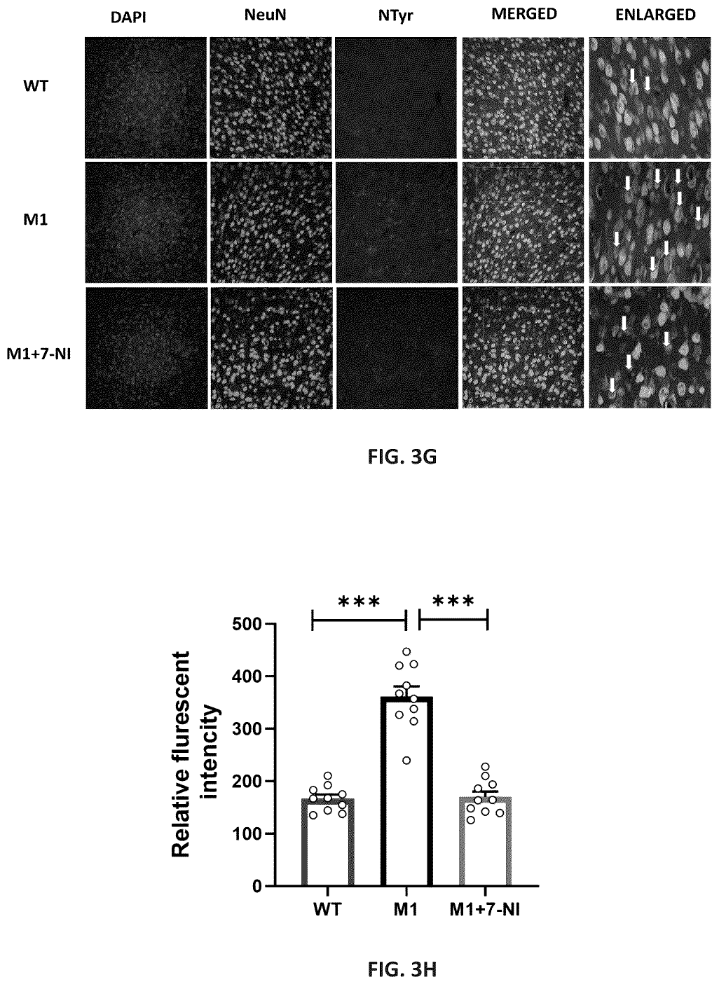

7-nitroindazole (7-NI) is a selective inhibitor of neuronal nitric oxide synthase. Mutant mice were administered with 7-NI interparentally (IP), 80 mg/kg/day for 7 days, as described hereinabove. To examine the effect of 7-NI administration on mice behaviors, a novel object recognition test (NOR) and sociability test were performed (See, FIGS. 4A-B), and the obtained data are presented in FIGS. 3A-B, respectively.

As shown in FIG. 3A, the NOR test showed that the 7-NI-treated mice exhibited a significant preference for the novel object over the familiar object, while vehicle-treated mice spent significantly less time exploring the novel object compared to the 7-NI treated mice.

As shown in FIG. 3B, the sociability test showed that 7-NI-treated mice spent significantly more time interacting with the stranger cage, while the vehicle-treated mice did not exhibit any preference for social interaction.

These data indicate that inhibiting the NO production and signaling could potentially reverse and attenuate the ASD phenotypes.

To further corroborate these findings, the expression of 3-nitrotyrosine in cortical tissue of the wild type (WT), compared with the SHANK3 mutant group (M1), and SHANK3 group treated with 7-NI (M1+7-NI), and in cortical tissue of the wild type (WT), compared with the CNTNAP2^{(-/-)} mutant group (M2), and the CNTNAP2^{(-/-)} mutant group treated with 7-NI (M2+7—NI) was tested, and the obtained data is presented in FIGS. 3C-D (for M1) and in FIGS. 3E-F (for M2).

As shown in FIGS. 3C-3F, the nitrosative markers of 3-Ntyr were significantly increased in the mutant mice of both models, whereby treatment with an nNOS inhibitor (7-NI) reduced the expression of 3-Ntyr in both mice models of ASD.

These findings demonstrate that NO and SNO are implicated in ASD pathogenesis, that treatment of 7-NI leads to reversal of nitrosative stress, and that 3-Ntyr can be used as an indicative marker for an activity of an nNOS inhibitor.

3-nitrotyrosine levels in were also evaluated by confocal microscopy of the cortical region in neurons of the tested mice, and the obtained data is presented in FIGS. 3G and 3H. As can be seen, 7-NI treatment reduced 3-nitrotyrosine levels in the neurons by about 50%, compared to neurons of untreated mice.

### Example 4

### Additional Behavioral Analyses in a C57BL/6 Mouse Model Following SNAP Administration

To further examine the potential effects of SNAP administration on mice behaviors, a large set of behavioral tests were conducted following the treatment of C57BL/6 wild-type mice (WT) with SNAP (interparental (IP), 20 mg/kg/day for 10 days), in accordance with the protocol described hereinabove.

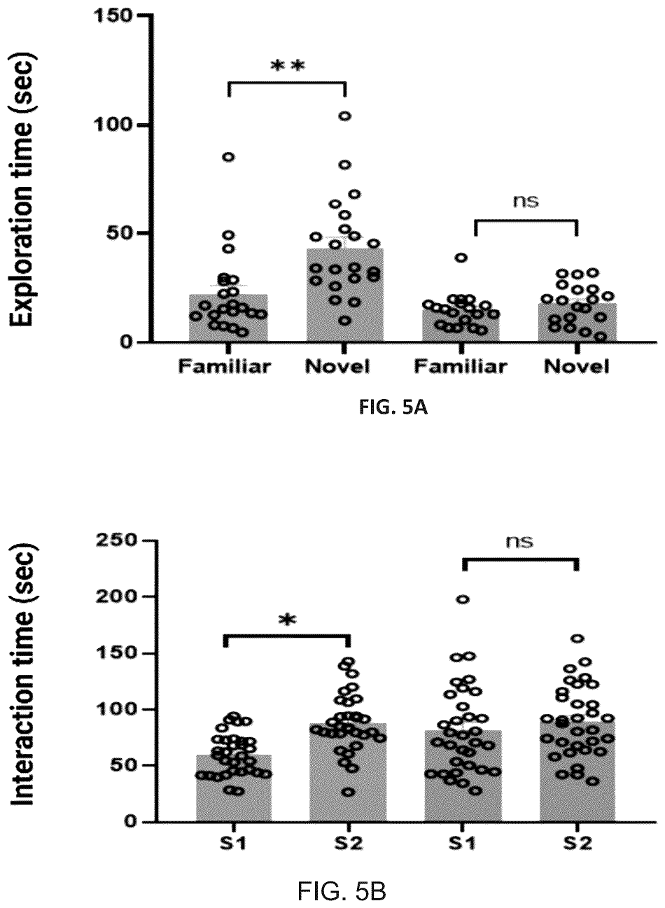

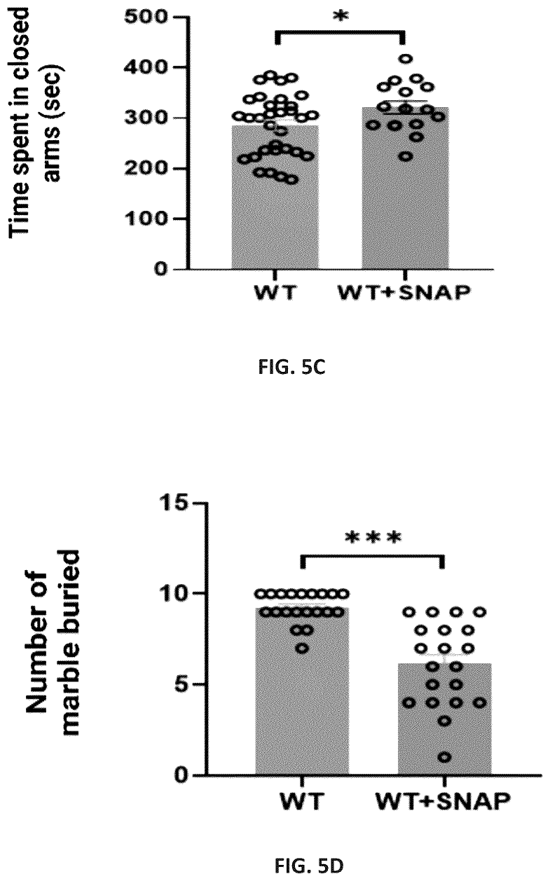

Initially, a novel object recognition test (NOR) was performed to evaluate the novelty seeking and the restricted interest in the mice. This test is based on the spontaneous tendency of the mice to spend a prolonged time exploring a novel object than a familiar one (see, FIG. 4A). As shown in FIG. 5A, the NOR test showed that the WT mice spent significantly more time exploring the novel object than the familiar object, whereas the SNAP-treated mice failed to display a preference for the novel object, indicating a lack of interest and reduced novelty seeking among the SNAP-treated group.

A three-chamber sociability test was conducted as in Example 2 (see also FIG. 4B), to evaluate the interest of the mice to engage in social interaction with a stranger mouse. Generally, the mice prefer to spend more time with another mouse (sociability), and the mice have the tendency to investigate a novel intruder more than a familiar one (social novelty). This test occurs in two different sessions: in the first one (see, upper illustration in FIG. 4B) the mice encounter a stranger mouse (Stranger) and an empty cage, and this session determines the sociability, while in the second session (see, lower image in FIG. 4B) the mice encounter the first intruder (S1) and a novel intruder (S2), and this session determines the social novelty.

The analysis showed that both the WT and the SNAP-treated mice spent more time interacting with the stranger mouse than with the empty cage (data not shown). As shown in FIG. 5B, the second session showed that the WT mice spent more time interacting with the stranger mouse (S2) than the familiar one (S1), indicating high sociability and social novelty among the WT mice. Conversely, the SNAP-treated mice did not exhibit any significant preference to engage in social interaction with the mice, possibly indicating impaired social novelty following the SNAP treatment.

An elevated plus maze test (see, FIG. 4C) was performed to evaluate the anxiety-like behaviors among the mice, typically the anxiety quantified as the time spent in the closed arms. As shown in FIG. 5C, the analysis showed that the SNAP-treated mice spent significantly more time in the closed arms compared to WT group, reflecting a likelihood of anxious behaviors among the mice that were treated with SNAP.

A marble burying test (as shown in FIG. 4D) was utilized as an indication for repetitive behaviors and restricted interests. As shown in FIG. 5D, the analysis showed that the SNAP treated mice buried significantly less marbles compared to their WT counterparts. These results indicate a lack of interest to explore novel objects in the SNAP-treated mice.

Collectively, these results suggest that high concentrations of NO could potentially induce behavioral and cognitive deficits similar to those observed in ASD. These include reduced novelty seeking, repetitive behaviors, impaired social novelty, and anxiety-like behaviors.

### Example 5

### Additional Behavioral Analyses in CNTNAP2 Mouse Model and SHANK3 Mouse Model Following 7-NI Administration

Behavioral tests as described in Examples 3 and 4 hereinabove were performed.

In one set of tests, the mice were divided into three testing groups: WT mice, SHANK3 knockout mice (M1) and SHANK3 knockout mice that were administered with 7-NI (M1+7NI) (chronical IP injections, 80 mg/kg/day for 10 days, 3 days before starting the behavioral test).

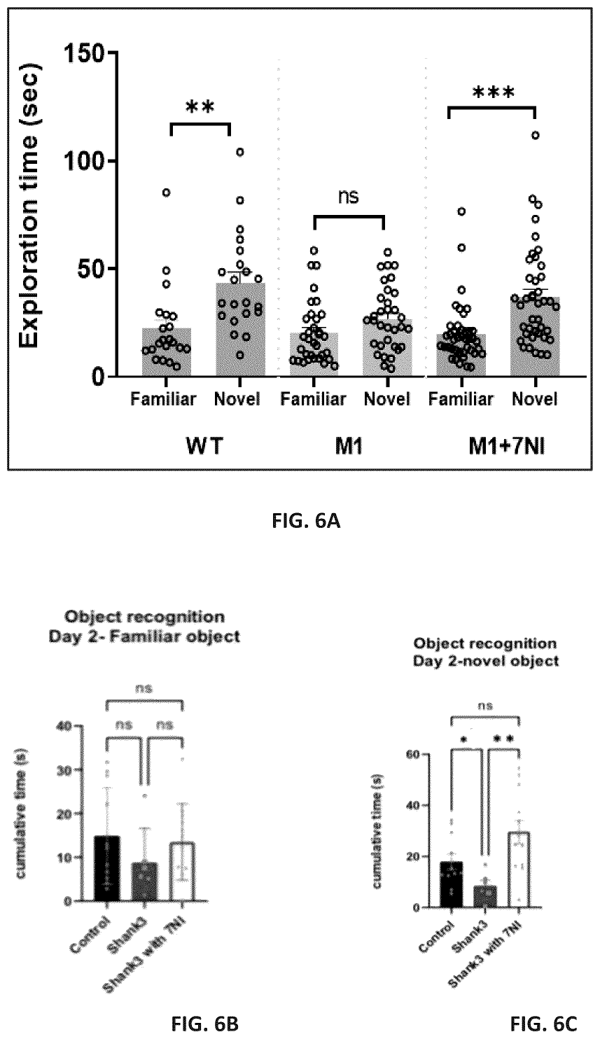

The data obtained in the novel object recognition test is presented in FIG. 6A, and show that the WT mice spent significantly more time exploring the novel object than the familiar one, whereas the M1 mice did not exhibit any significant preference for the novel object, reflecting a reduced novelty seeking and restricted interests. Following treatment with the 7-NI the M1 mice exhibited a significant preference for the novel object over the familiar one.

In another set of tests, the Novel Object Recognition Test (see, FIG. 4A) was conducted with aged (10 months) WT mice, SHANK3 knockout mice (M1) and SHANK3 knockout mice that were administered with 7-NI (M1+7NI) (chronical IP injections, 80 mg/kg/day for 10 days, 3 days before starting the behavioral test).

The obtained data is shown in FIGS. 6B and 6C and show that also the aged autism mice lost their memory, which was restored by the 7-NI treatment.

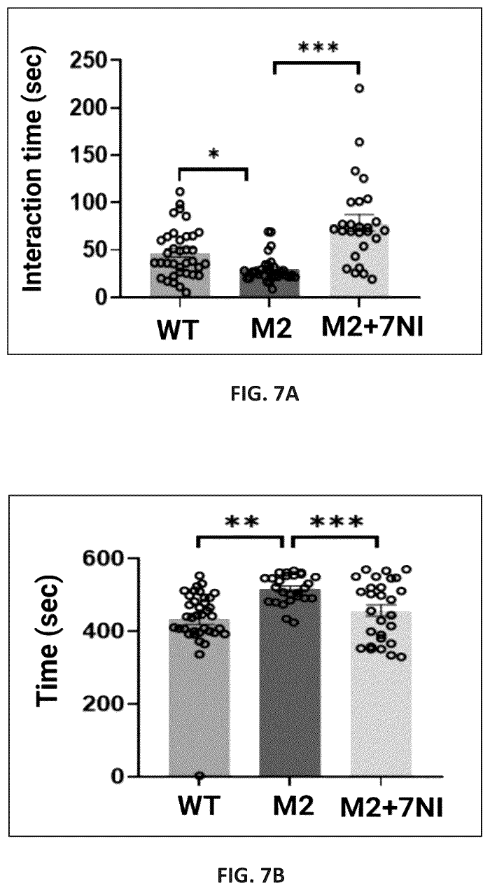

In another set of tests, the potential effects of inhibiting NO on the cognitive functions was assayed on an additional mouse model of ASD-CNTNAP2−/− mutant mice. The mice were divided into three testing groups: WT mice, CNTNAP2−/− mutant mice (M2), and mutant mice that were administered with 7-NI (M2+7NI). A three-chamber sociability test was conducted (See, FIG. 4B) and the data is presented in FIG. 7A. The analysis showed that the mutant mice (M2) spent significantly less time interacting with the stranger mouse compared to their WT littermates, indicating a lack of sociability among the mutant mice. The 7-NI treated mice (M2+7NI) spent significantly prolonged time interacting with the stranger mouse compared to their untreated counterparts-M2. An elevated plus maze test (see, FIG. 4C) was also conducted and the obtained data is presented in FIG. 7B. The analysis showed that the mutant mice (M2) spent significantly more time in the closed arms compared to the WT mice, conceivably, indicating increased anxiety among the mutant mice. Following the treatment with 7-NI, the mice exhibited less anxiety like behaviors compared to the untreated mutant mice. Collectively, these results suggest that the autistic phenotypes observed in the SHANK3 mutant mice may potentially reversed through the inhibition of NO production.

### Example 6

### Dose Dependent Effect of 7-NI Treatment of SHANK3 Mice

A dose dependent study was conducted to determine the optimal dose that may produce the greatest effect on mice behavior. The SHANK3 mutant mice (n=5 for each group) were treated with the 7-NI (i.p) at doses of 0, 20, 50, and 80 mg/kg. This was followed by a set of behavioral tests to assess the proper dose of 7-NI that can reverse the autistic phenotypes observed in this model.

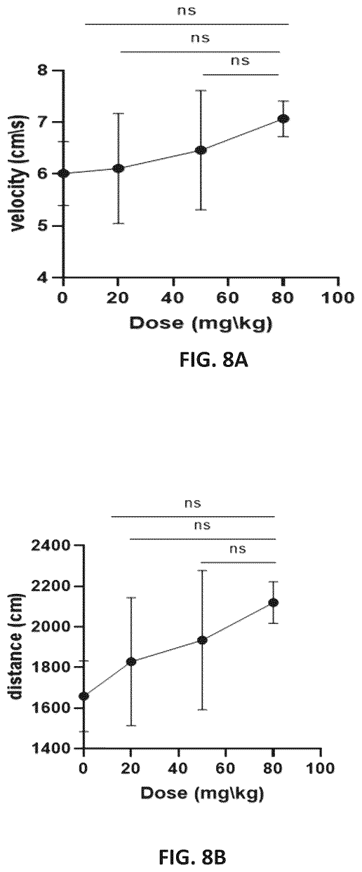

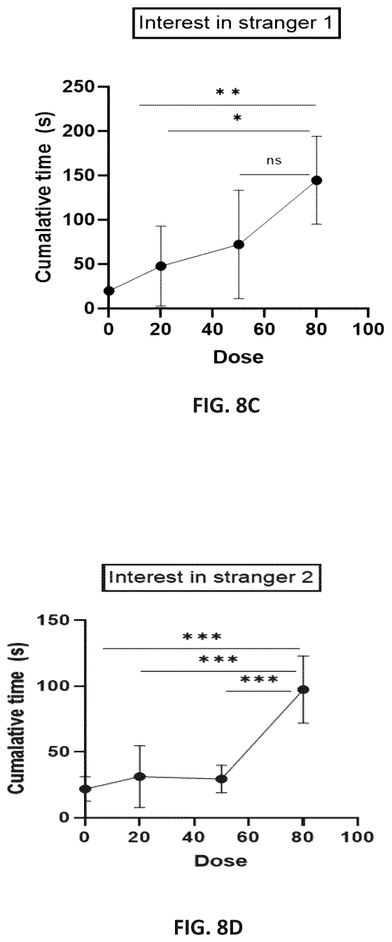

Initially, an open field test was performed to examine the general motor activity levels in mice that is measured by the distance travelled and velocity. As shown in FIGS. 8A-B, no significant difference in the motor activity was observed among the tested groups following treatment with 20, 50 and 80 mg/kg. This indicates that higher doses of 7-NI do not affect the locomotion of the mice.

A three-chamber sociability test was conducted to evaluate the interest of mice in engaging in social interaction with a stranger mouse, as described hereinabove and shown in FIG. 4B. As shown in FIG. 8C, mice that were treated with 80 mg/kg spent significantly more time interacting with the stranger mouse compared to the mice that were administered with 20 mg/kg of 7-NI. Comparing to the 50 mg/kg, although statistically the difference is not significant, the 80 mg/kg-treated group spent more time interacting with the stranger mouse.

As shown in FIG. 8D, the second session of the test showed that the mice that were treated with 80 mg/kg 7-NI spent more time interacting with the novel mouse (S2) compared to their counterparts that were treated with lower doses of 7-NI.

Overall, these results indicate that 80 mg/kg of 7-NI yielded the greatest reversal of the autistic phenotypes including impaired social behaviors.

### Example 7

### siRNA Silencing of nNOS in the Background of Silenced SHANK3 Alleviates ASD Phenotype as Determined by the Synaptophysin Marker

A cell culture model for ASD was developed by silencing SHANK3 with an siRNA (5-3 RNA)-CGA UGA UAA GCA GUU UGC AAA GCU U (SEQ ID NO: 3) in the neuronal cell line SHSY-5Y cells, followed by silencing with of nNOS using the following RNA (5-3) CCU UGU CCA ACA UGC UCC UAG AGA U (SEQ ID NO: 4).

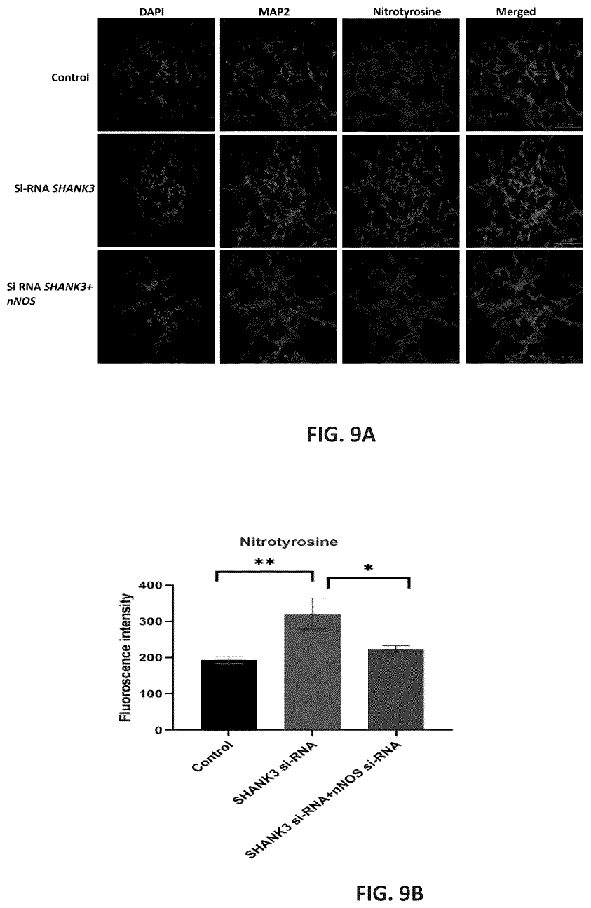

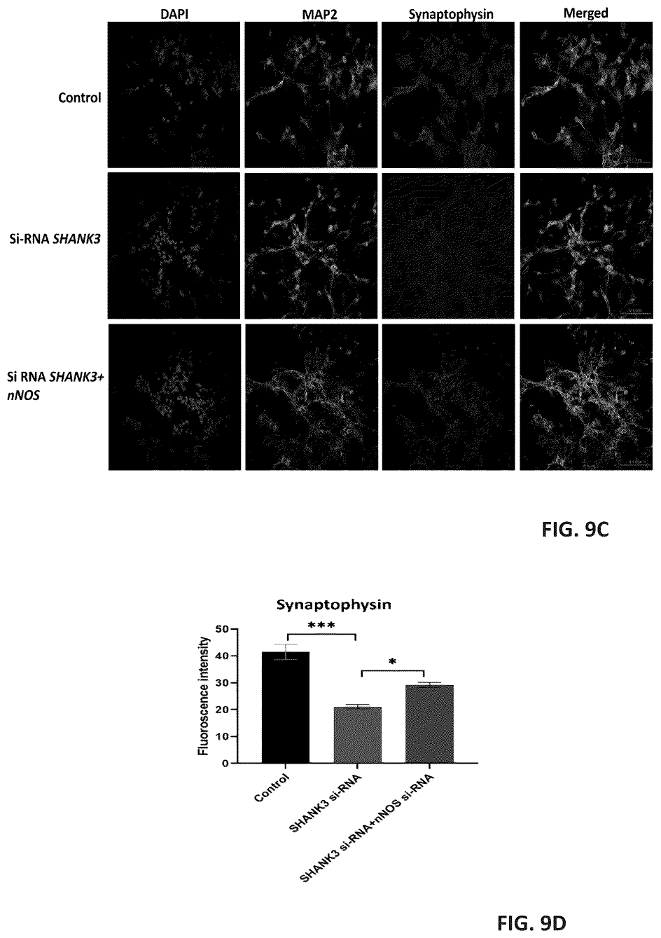

SHSY-5Y Cells (with or without treatment) were fixed post treatment in 4% paraformaldehyde, incubated with the Primary rabbit MAP2+ primary mouse monoclonal 3-nitrotyrosine (FIGS. 9A-B) and with Primary rabbit monoclonal synaptophysin+primary mouse Map2 (FIGS. 9C-D). Thereafter, cells were incubated with anti-mouse secondary Alexa fluor488 (green)/594 (red), and anti-rabbit secondary Alexa fluor 488 (green)/594 (red), conjugated secondary antibodies. After washing, cells were mounted with DAPI (Blue). The images were acquired using a confocal microscope. Each group contained nine independent sets of experiments (initial cell seed number—about 10⁶).

To support the effect of nucleic acid silencing on a SHANK3 silenced background, the present inventors applied siRNA to nNOS on primary cultures of SHSY-5Y Cells. Silencing nNOS in the SHANK3 si-RNA group (double knockdown) shows the reduction in nitrotyrosine production (FIGS. 9A-B). Synaptophysin, which is a reliable marker (Kathuria et al. Mol Psychiatry, 2018 March; 23 (3): 735-746) of synaptogenesis and ASD was found to be downregulated in SHSY-5Y cells after treatment with SHANK3 si-RNA (FIGS. 9C-D). When knocking down the nNOS in the SHANK3 si-RNA group (double knockdown), a recovery in synaptophysin protein expression was observed (FIGS. 9C-D).

### Part II

### Exemplary Formulations

7-nitroindazole (7-NI) inhibits nitric oxide synthase (NOS) and has been used extensively as a selective inhibitor of neuronal NOS (J. Neurol. Sci. 1998, 160, 9-15). Compared to the other drugs that are currently available, 7-NI evidences a 10-fold selectivity for neuronal NOS (Pharm Res., 2001, 18, 1607-1612). Despite of the high selectivity for the NOS, 7-NI has a short half-life that does not exceed 2 hours even in very high doses (Biopharm. Drug Dispos. 2000, 21, 221-228). The use of 7-NI is further limited by a very low solubility in aqueous medium, and hence it is commonly administered by IP injection upon dissolving it in oils (e.g., peanut oil or corn oil). Thus, in order to keep the optimal levels in the animals, a daily high drug dose of 80 mg/kg or more is required.

A need therefore exists to design improved 7-NI (or derivatives or analogs thereof) formulations that can allow oral, nasal, sublingual, or buccal administration of the 7-NI, or which act as extended release formulation which provide a constant release of the 7-NI in biological medium for 10 days or more.

The following Examples describe exemplary formulations, designed by the present inventors so as to facilitate the use of 7-NI in the context of the present embodiments.

### Example 8

### 7-Nitroindazole Pro-Nanodispersion Lipid Formulation (PNL) for Injection and Oral Delivery

Blank PNL formulation is generally prepared by mixing common surfactants/dispersing agents, particles-forming lipids, and an edible solvent.

Table 1 below presents an exemplary PNL formulation according to some embodiments of the present invention. The components listed in Table 1 and the amount/concentration thereof should not be regarded as limiting as each of the recited components can be replaced by an alternative component that exhibits the same functionality and is considered as safe, and the relative amount of each component can be altered or adjusted to improve solubility and/or stability.

TABLE 1

| Component | Function | % (w/w) |
|---|---|---|
| Tween ®20 | hydrophilic dispersing agent/ | 10-20 |
| | surfactant | (e.g., about 14) |
| Span 80 | hydrophobic dispersing agent/ | 10-20 |
| | surfactant | (e.g., about 14) |
| Lechitin | Natural, neutral phospholipid for | 5-10 |
| | particle formation | (e.g., about 8) |
| Tricaprin | solid lipid core material | 10-20 |
| | | (e.g., about 14) |
| PEGylated | Dispersing agent and particle | 10-20 |
| Hydrogenated | formation | (e.g., about 14) |
| castor oil | | |
| (HCO 40) | | |
| Ethyl lactate | Water miscible solvent that dissolve | 30-40 |
| | all ingredients that induce | (e.g., about 35) |
| | nanoparticle formation | |

The formulation can be prepared by mixing the components at room or elevated temperature to form a clear liquid which 7-nitroindazole is soluble in at about 20% w/v.

The formulation is a liquid or semi-solid at room temperature and can optionally be loaded in soft gelatin capsules for oral swallowing, or formulated as tablets for oral administration, or mixed with water to form a nano-dispersion for administration by nasal spray, inhalation, eye drops or injection, as described herein.

In an exemplary procedure, tablets containing 7-nitroindazole-PNL were prepared using the formulation described in Table 1. To a porcelain mortar, to which 300 mg of silica powder, Neusilin US2, was added along with 200 microliters of PNL formulation containing 40 mg 7-NI. The mixture was thoroughly blended with a mortar and a pestle until a homogenous mixture was obtained. Thereafter, 100 mg HPMC and a tableting aid Syloid 244 FT (5 mg) were added in a geometric mixing until a homogenous mixture was obtained. The powdery mixture was fed manually into the die of an instrumented single punch tableting machine to produce tablets using a 1.3 cm flat faced punch. Tablets were prepared by direct compressing. The compression force was set at 3 ton for 10 seconds.

### Example 9

### Exemplary 7-Nitroindazole Pro-Nanodispersion Lipid Formulation (PNL) for Oral, Nasal and Ophthalmic Delivery or 7-NI

An exemplary PNL formulation for oral delivery of 7-NI was prepared using MCT, Tween®80; Kollipor RH 40; Kollipor HS 15; Propylene glycol; Labrasol®; and 7-Nitroinidazole (7-NI), as follows:

MCT: Medium chain triglycerides (MCTs) are partially man-made fats. The name refers to the way the carbon atoms are arranged in their chemical structure. MCTs are generally made by processing coconut and palm kernel oils in the laboratory. Usual dietary fats, by comparison, are long-chain triglycerides. MCTs are also used as medicines. MCTs are taken by mouth or given with a needle alone or along with usual medications for involuntary weight loss in people who are very ill (cachexia or wasting syndrome). MCTs are also used for obesity, seizures, athletic performance, and other conditions, but there is no good scientific evidence to support these other uses.

Tween®80: Polysorbate 80 is a nonionic surfactant and emulsifier often used in foods and cosmetics. This synthetic compound is a viscous, water-soluble yellow liquid.

Labrasol®: Labrasol is a PEG derivative of medium chain fatty acid triglyceride of capric and caprylic acid (Caprylocaproyl Polyoxyl-8 glycerides). It consists of a small fraction of mono-, di- and triglycerides and mainly PEG-8 (MW 400) monoester and diester of caprylic (C8) and capric (C10) acids. It is widely used in PNL formulations due to its good solubilizing power and spontaneous self-emulsification ability.

Kolliphor® RH 40: Kolliphor RH 40 is a nonionic solubilizer and emulsifying agent obtained by reacting 40 moles of ethylene oxide with 1 mole of hydrogenated castor oil. The main constituent of Kolliphor®RH 40 is glycerol polyethylene glycol oxystearate, which, together with fatty acid glycerol polyglycol esters, forms the hydrophobic part of the product. The hydrophilic part consists of polyethylene glycols and glycerol ethoxylate. Kolliphor®RH 40 is a white to yellowish paste at 20° C. Kolliphor®RH 40 is widely used and approved solubilizer, is chemically stable and practically tasteless, making it a good component for oral applications, particularly suited for aqueous solutions of fat-soluble vitamins, Suitable for preparing dispersions and emulsions as it is less digestible in the gastrointestinal tract as compared to other unsaturated ethoxylated surfactants.

Kolliphor® HS 15: Kolliphor® HS 15 is a nonionic solubilizer and emulsifying agent obtained by reacting 15 moles of ethylene oxide with 1 mole of 12-hydroxy stearic acid. Kolliphor® HS 15 consists of polyglycol mono- and di-esters of 12-hydroxystearic acid (=lipophilic part) and of about 30% of free polyethylene glycol (=hydrophilic part). The free polyethylene glycol can be determined by HPLC. Kolliphor® HS 15 was designed to provide a low immunogenicity during parenteral administration.

Propylene glycol: Propylene glycol is a synthetic food additive that belongs to the same chemical group as alcohol. It is a colorless, odorless, slightly syrupy liquid that is a bit thicker than water. It has practically no taste.

Table 2 below presents the components that make-up the exemplary PNL blank formulation.

TABLE 2

| Component | Function | % (w/w) |
|---|---|---|
| Tween ®80 | Hydrophilic surfactant | 20 |
| MCT | Lipid core | 10 |
| Kollipor ®RH 40 | emulsifier | 20 |
| Kollipor ®HS 15 | emulsifier | 10 |
| Labrasol ® | Lipidic dispersing agent | 20 |
| Propylene glycol | Water-miscible solvent | 20 |
| Sum | | 100.0 |

All the materials were weighed in the glass vial and heated at 70° C. for 20 minutes until a clear transparent liquid was obtained. The mixture was stirred timely to assure complete mixing and homogeneity. Thereafter, the mixture was cooled to room temperature for 2 hours.

7-NI loaded PNL formulation was prepared by adding the drug at a selected weight % to the PNL mixture at 70° C. and stirring for 30 minutes. The drug dissolved in the PNL mixture. The mixture thereafter was cooled to room temperature for 1 hour.

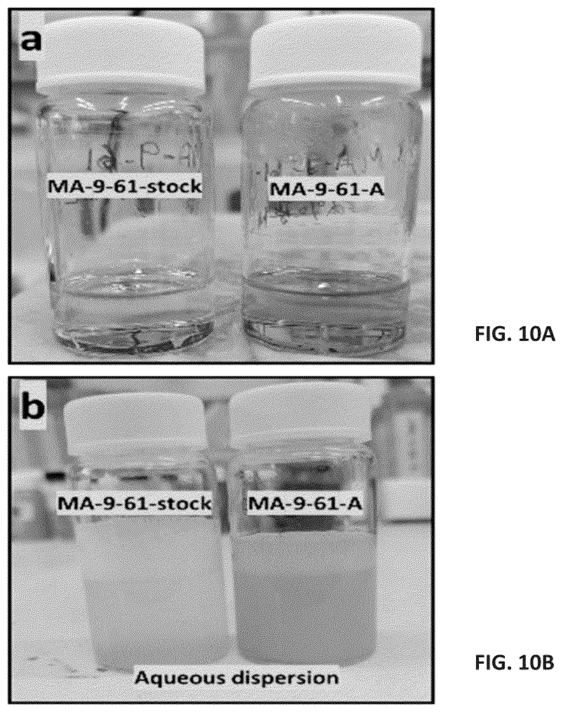

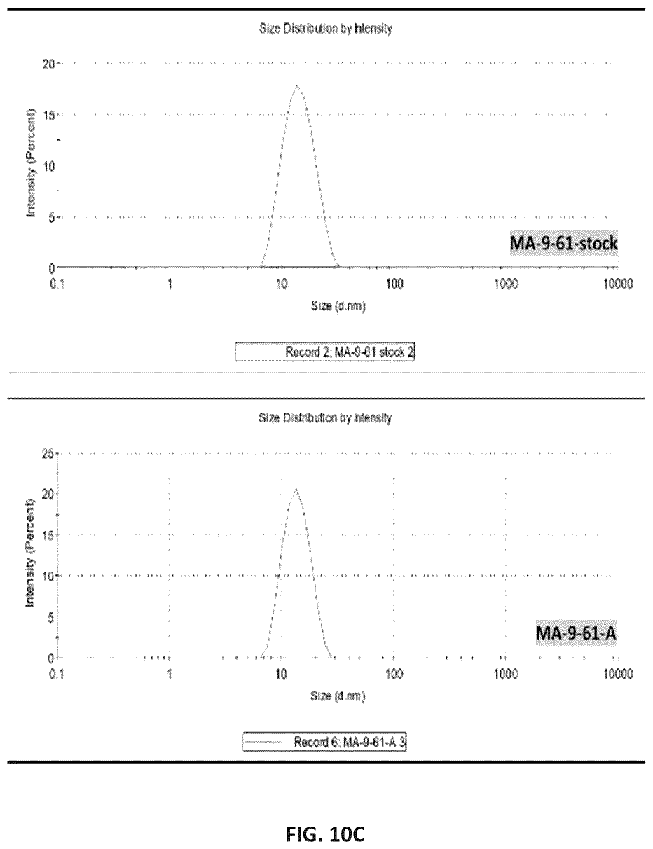

FIG. 10A presents photographs of the obtains PNL solution, without and with 7-NI, showing that both formulations are transparent at room temperatures.

The stability of the PNL was tested by keeping it at room temperature for 3 days. No phase separation was observed.

Both PNL formulations, with and without 7-NI dispersed instantly in 37° C. water, upon hand-shaking, as shown in FIG. 10B.

Dynamic light scattering (DLS) was used to check the size of the obtained nanoparticle aqueous dispersions. 100 μL of the freshly prepared PNL was dispersed in 10 mL DDW, and 100 μL of the dispersion was diluted with 1 mL DDW and analyzed.

Dynamic Light Scattering (DLS) was performed to determine the size of the nanoparticles using a Zetasizer (Nano ZS, Malvern Instruments, UK) equipped with inbuilt software. Nearly 1 mL of the nanoparticle dispersion in buffer was placed into a glass cuvette (12 mm glass cell with square aperture, cell type: PCS1115). Each NP batch was appropriately diluted with the appropriate amount of buffer. The measurement angle was selected at 173° Backscatter (NIBS) in instrument automatic selection mode.

The z-average of the NPs was calculated using the auto-correlation function of the intensity of light scattered from the particles, assumed to be in the spherical form by the instrument software.

All measurements were conducted in triplicate at 25° C. for each batch of NPs.

The obtained DLS data is presented in Table 3 below and in FIG. 10C. As can be seen, no substantial difference between the formulation without 7-NI and the formulation with 2% w/w 7-NI is observed.

The stability of the aqueous PNL dispersion was checked after 3 days storage at room temperature. The obtained data is shown in Table 3 below. No sedimentation and no significant change in the size of the nano-dispersion of the 7-NI containing PNL dispersion was observed upon storage.

TABLE 3

| | 7NI w/w | Dispersion | | | | |
|---|---|---|---|---|---|---|
| Sample | % | storage time | | Size (nm) | PDI | Zeta (mV) |
| MA-9-61-stock | — | 2 | hours | 13.21 ± 0.24 | 0.092 ± 0.014 | −5.87 ± 5.38 |
| MA-9-61-A | 2% | 2 | hours | 13.16 ± 0.05 | 0.039 ± 0.004 | −5.46 ± 2.63 |
| MA-9-61-stock | — | 3 | days | 16.06 ± 0.29 | 0.100 ± 0.012 | — |
| MA-9-61-A | 2% | 3 | days | 15.76 ± 0.23 | 0.127 ± 0.016 | — |

The dispersibility of the PNL formulations upon storage at room temperature for 3 days was also tested. After 3 days storage, 100 μL of the PNL formulation was dispersed in 10 mL DDW and checked for aggregation. The obtained data is presented in Table 4, demonstrating no aggregation in both PNL formulations.

TABLE 4

| | 7NI | | |
|---|---|---|---|
| Sample | w/w % | Size (nm) | PDI |
| MA-9-61-stock | — | 24.63 ± 0.27 | 0.185 ± 0.007 |
| MA-9-61-A | 2% | 17.43 ± 0.34 | 0.174 ± 0.010 |

The obtained data demonstrate that the designed 2% w/w 7-NI-containing PNL formulation is a stable formulation and does not show aggregation upon dispersion in DDW. The PNL nano-dispersion is easily prepared by shaking in water and the size of the particles in the dispersion is smaller than 20 nm. The designed 7-NI-loaded PNL formulation is suitable for the oral drug delivery of 7-NI as well as for eye drops or nose-to-brain nasal spray for direct delivery to the brain.

### Example 10

### Exemplary Polymeric Microparticles Loaded with 7-Nitroindazole for Extended Release Injectable Formulations

### PLGA 75/25 Microparticle Preparation:

100 mg PLGA 75/25 (100 kDa), 5 mg PLGA 75/25 (14 kDa) and 25 mg 7-nitroindazole are dissolved in 5 mL dichloromethane. The organic mixture is added dropwise to 100 mL of 0.2% PVA aqueous ammonia solution with constant stirring. The mixture is stirred for 5 hours at room temperature until all the organic solvent is evaporated and then ethanol is added to precipitate the particles. The solvent is decanted out and the microparticles are washed 2 times with water and then lyophilized with 0.1 mL amount of 0.1% (w/v) Pluronic®127. Particles loaded with about 20% w/w 7-nitroindazole are obtained with an average particle size of about 35 microns. The in vitro release is determined in physiologic phosphate buffer at 37° C. where the release media is replaced at certain time points and analyzed by UV or HPLC for 7-nitroindazole concentration. A constant release for more than 20 days is obtained.

### Example 11

### 7-Nitroindazole Salts Preparation Procedures

The indazole moiety of 7-Nitroindazole is prone to the formation of a salt by: (i) eliminating the proton from the N—H of the azole to thereby obtain a negatively-charged indazole that forms a salt with a positively charged moiety (e.g., cation), including, for example, divalent and trivalent metal ions, ammonium ions and phosphonium ions, referred to herein as cationic salts of 7-NI; or (ii) protonating the basic amine that is part of the aromatic ring using proton containing molecules, such as, for example, HCl, phosphoric acid, phosphonic acid, sulfuric acid, sulfonic acid, and organic acids, referred to herein as anionic salts of 7-NI. Such salts may confer different or improved properties compared to 7-NI, including, for example, solubility, thermal properties and stability, chemical stability, ease of formulation, controlled release, crossing biological membranes and bio-distribution.

The following describes the preparation of exemplary salts of 7-NI. Similar salts can be prepared with analogs or derivatives of 7-NI as described herein.

The salts can be formulated in, for example, aqueous or lipidic solutions for injection, oral administration, or nasal-spray administration.

For example, salts that are formed by abstraction of the N—H proton of the Indazole to form Indazole anion that form a salt with cationic counterparts (a cationic salt) can be prepared using various cation-forming materials that provide a desirable solubility. Thus, solubility can be tuned by the selecting the type of salt. For example, lipidic cation such as phosphatidyl choline will form a lipid-soluble 7-NI. When PEG terminated with triethylammonium forms a salt with indazole anions, the hydrophilic PEG chain will make the molecule hydrophilic.

### Cation Salts of 7-Nitroindazole:

Salts with cations counterpart are prepared by reacting a corresponding base with 7-nitroindazole. The salt is analyzed by FTIR, DSC/TGA thermal analysis, elemental analysis and/or NMR.

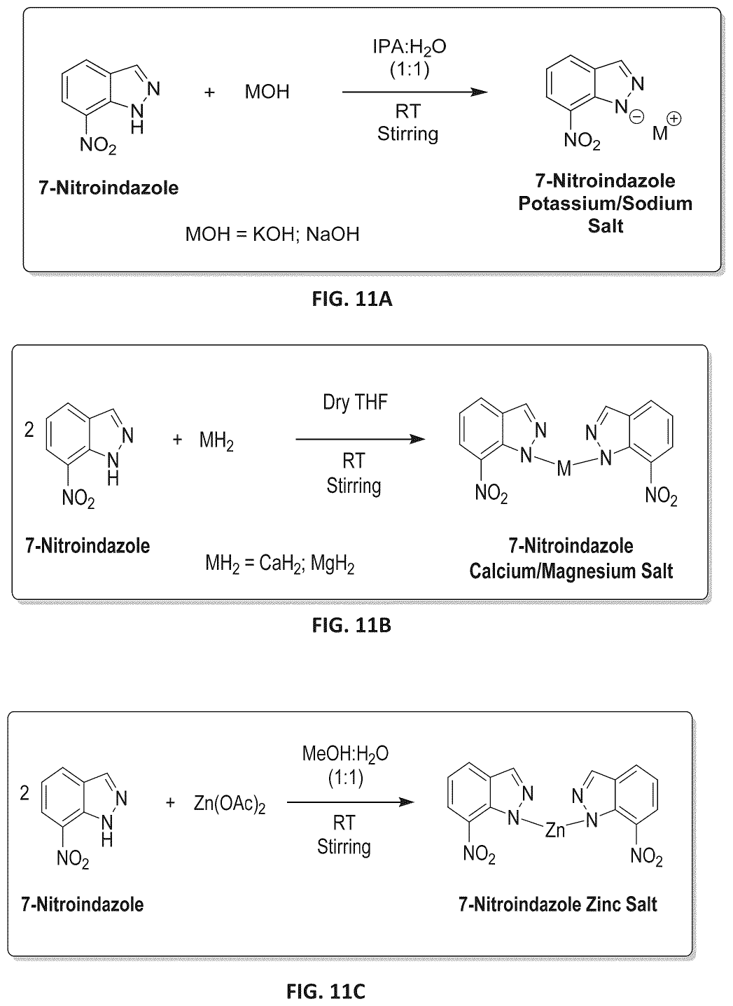

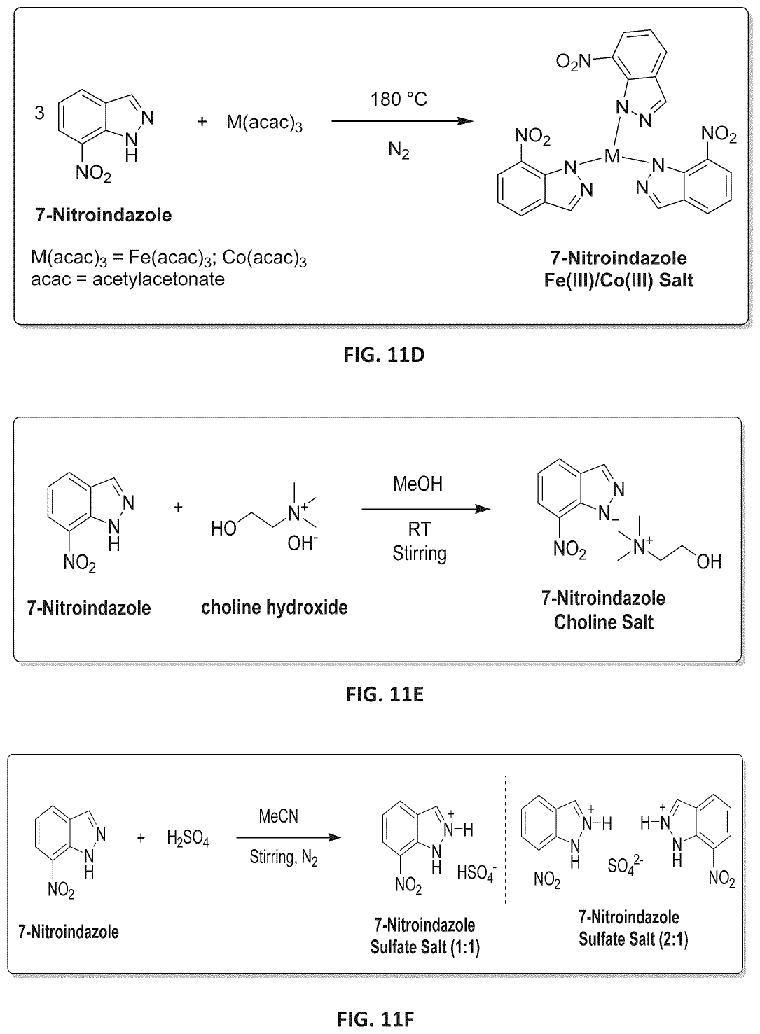

Potassium/Sodium salts of 7-Nitroindazole (FIG. 11A): Potassium/Sodium salts of 7-nitroindazole are synthesized using the synthetic procedure for potassium salt of indazole or triazole or pyrazole [7-9]. To a mole equivalent of 7-nitroindazole dissolved in water and 2-propanol (1:1) mixture, a mole equivalent of 1 M aqueous potassium/sodium hydroxide is added and stirred for 15 minutes. The solution is evaporated to dryness under vacuum at 80° C. and then further dried in a vacuum oven for 4 hours at 110° C.

Calcium/Magnesium salts of 7-Nitroindazole (FIG. 11B): Calcium/Magnesium salts of 7-nitroindazole are synthesized using 7-nitroindazole and calcium/magnesium hydride. To a solution of 7-Nitroindazole (2 mol equivalents, at least) dissolved in dry THF, CaH₂/MgH₂ is added and the obtained reaction mixture stirred overnight. The solution is thereafter evaporated to dryness under vacuum.

Zinc salt of 7-Nitroindazole (FIG. 11C): Zinc salts of 7-nitroindazole are synthesized using the synthetic procedure for zinc salt of pyrazole [10]. To a solution of Zn(OAc)₂ (1 mol equivalent) in H₂O, a solution of 7-nitroindazole (2 mol equivalents, at least) in MeOH is added under stirring. The obtained suspension (e.g., 1:1 H₂O:MeOH mixture) is stirred for 48 hours and thereafter filtered. The obtained solid salt is washed with water and dried under vacuum.

Iron/Cobalt salt of 7-Nitroindazole (FIG. 11D): Iron(III) or Cobalt(III) salt of 7-nitroindazole is synthesized using the synthetic procedure for Fe(III) or Co(III) salt of pyrazole [11]. A mixture of Fe(acetylacetonate)₃ (Fe(acac)₃) (1 mol equivalent) or Co(acac)₃ (1 mol equivalent) and excess 7-nitroindazole (e.g., 15 mol equivalents) is heated in a sealed flask under nitrogen for 6 hours at 180° C. without stirring. The mixture is allowed to cool, and the precipitated solid is washed with acetone in order to remove excess 7-nitroindazole and then filtered off, giving the Fe(III)/Co(III) salt of 7-nitroindazole. The acetone can be evaporated to recover the unreacted 7-nitroindazole.

Choline salt of 7-Nitroindazole (FIG. 11E): Choline salt of 7-nitroindazole in synthesized using 7-nitroindazole and choline hydroxide. To a mol equivalent of 7-nitroindazole dissolved in methanol, a mol equivalent of choline hydroxide is added and the obtained solution stirred overnight. The solution is thereafter evaporated to dryness in vacuum and then further dried in a lyophilizer.

### Anion Salts of 7-Nitroindazole:

Sulfate salt of 7-Nitroindazole (FIG. 11F): Sulfate salt of 7-nitroindazole is synthesized using the synthetic procedure for sulfate salt of pyrazole [12-15]. 7-Nitroindazole (1 or 2 mol equivalent(s)) is dissolved in acetonitrile or another organic non-protic solvent and then concentrated H₂SO₄ (1 mol equivalent) is added dropwise under cooling conditions. The reaction mixture is stirred for 24 hours and refluxed in the presence of a flow of nitrogen gas. The obtained solid is washed with diethyl ether three times and dried in a vacuum oven overnight.

Phosphate and mesylate salts of 7-NI are prepared similarly. The phosphate counter ion can be replaced with an organic carboxylate, including, for example, acetic, fumaric, and hexanoic acid.

### Example 12

### Polymeric Gel Formulations for Controlled Release of 7-NI

The present inventors have designed biodegradable-polymer based formulations that, once administered, provides a constant release of 7-NI in biological medium for 10 days or more.

An exemplary formulation is based on a polymeric gel carrier that comprises poly(sebacic-co-ricinoleic) acid (PSARA) gel.

### Preparation of 7-NI-Loaded Polymeric Gel:

PSA: RA pasty injectable polymer of a 9200 molecular weight was prepared as previously prepared [Journal of Controlled Release, 2017, 257, 156-162]. The formulation of PSA: RA with 7-NI was prepared with the drug at room temperature by gravimetric mixing, where 7-NI fine powder is gradually added to the polymer paste and mixed well to form a uniform fine paste. The paste is loaded into syringes for treatment. The final composition of 7-NI in the PSA: RA formulation was 5% w/w. The formulation is injectable through 19G or 21G needle.

### Determination of 7-NI in the Polymeric Gel:

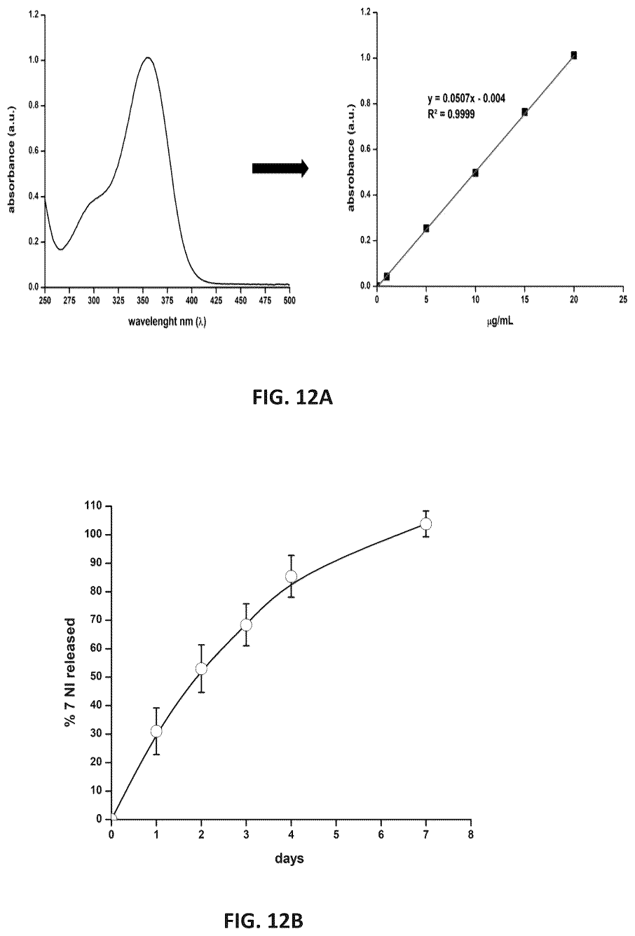

Quantification of the 7-NI was done in methanol by detecting the absorbance wavelength at 355 nm. A standard curve of 7-NI in methanol at a concentration range of 1-20 μg/mL, as shown in FIG. 12A, was generated.

The release of 7-NI was monitored in 10 mM phosphate buffer at pH 7.2 and 37° C. under constant shaking at 75 rpm. The solubility of the 7-NI in the PBS was found to be 0.14 mg/mL, which is consistent with the literature reported solubility (see, for example, Biopharm. Drug Dispos. 2000, 21, 221-228). The release medium was diluted suitably with methanol and absorbance was recorded.

A small amount of the 7-NI gel formulation was placed in a 20 mL glass vial and 15 mL of the PBS were added thereto. The vial was then heated to 37° C. and the release of the 7-NI was determined based on the absorbance spectra recorded with time. At each time point all the released media was replaced with a fresh buffer. The samples were prepared in triplicates. FIG. 12B presents the release profile of the 7-NI from the polymeric gel formulation. As can be seen, the release of 7-NI lasted for 7 days in PBS.

### Example 13

### Effects of Poly (Sebacic-Co-Ricinoleic) Acid (PSARA) Gel Formulation Loaded with 7NI on SHANK3 Mouse Module Behavior

7-NI loaded PSA: RA injectable formulations for extended release of the high concentration of 7-NI were prepared as described in Example 12 hereinabove.

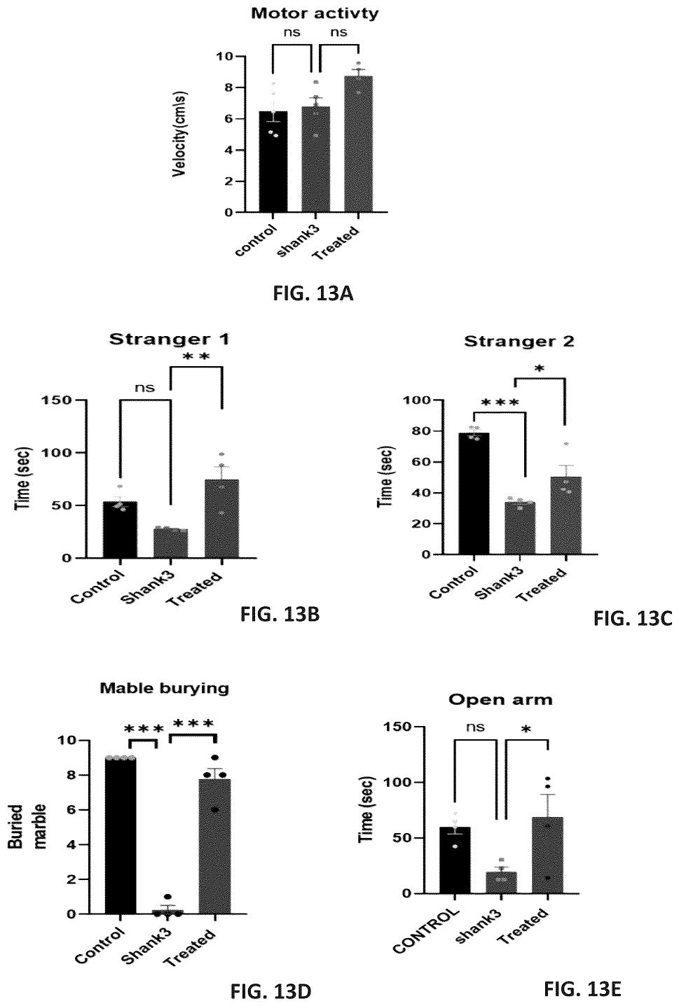

A 100 μL injection of the poly(sebacic acid-ricinoleic acid) (PSARA) gel formulation with 7NI was used to evaluate its effect in reversing ASD-like behaviors in SHANK3 mice. The motor activity, social interaction, anxiety and interest behaviors were examined using the behavior analyses as described herein. The obtained data is presented in FIGS. 13A-E.

As shown in FIG. 13A, in an open field test performed on day 4 after injection, there was no significant change in motor activity between treated mutant mice and the respective control groups. As shown in FIGS. 13B-C, in the three-chamber sociability test (day 9+10), both in the first and second days the treated mice spent more time around the cage of a novel animal/object compared to the untreated ones, indicating that 7-NI has a reversal effect on sociability behavior.

As shown in FIG. 13D, in the marble burying test (day 7), it was shown that while the mutant mice didn't bury marbles which could indicate the loss of interest, the treated ones buried in average 8 out of 10 marbles, indicating that 7-NI treatment enhances the mice interest. As shown in FIG. 13E, the anxious behavior for these mice was determined in an elevated plus maize and the obtained results show that the treated mice spent significantly more time in open arm comparing to the untreated mice.

### Part III

### Anti-Neural Cancer Effect of nNOs Inhibition

### Materials and Methods

### SH-SY5Y Cell Cultures and Drug Treatment:

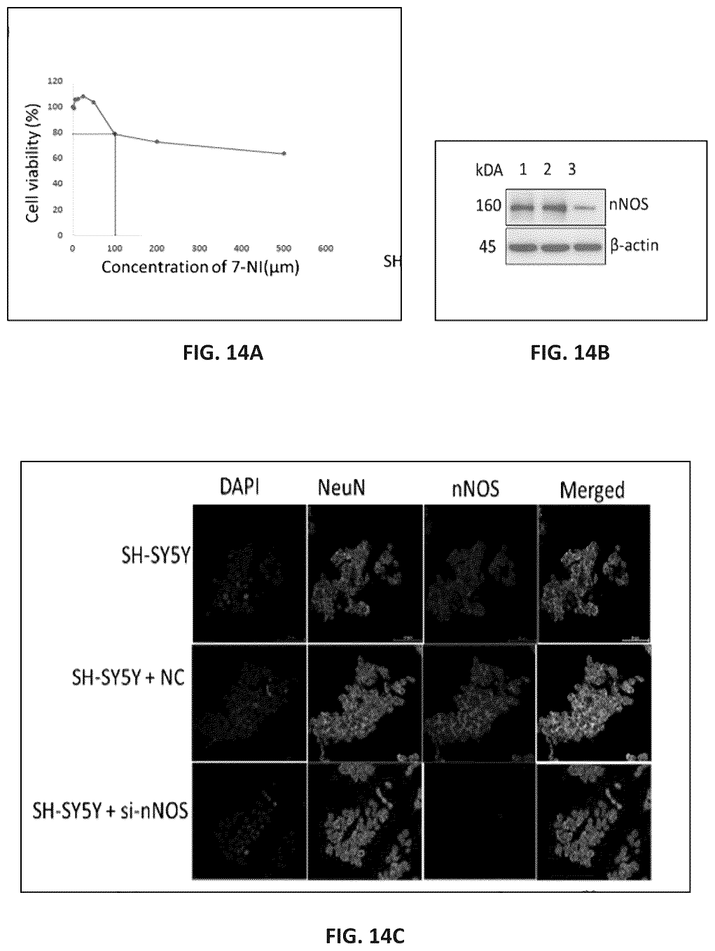

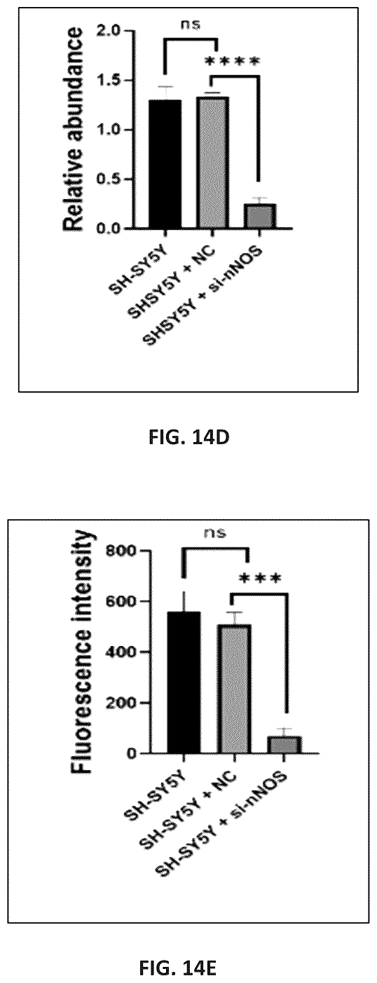

Human NB SH-SY5Y cells were obtained from the American Type Culture Collection (ATCC) and maintained in Dulbecco's modified Eagle's medium (DMEM) and Ham's F-12 (1:1) supplemented with 10% fetal bovine serum, 2 mM L-glutamine, and 100 U/ml penicillin-G/100 mg/ml streptomycin in a humidified atmosphere (37° C., 5% CO₂). Cells were plated onto 25 cm² and 75 cm² plates at a density of 7.0×10⁵ and 2.1×10⁶ cells/cm² respectively for Western blotting (WB) and 1×10⁵ for immunofluorescence. The SH-SY5Y cells were treated with 100 μM 7-nitroindazole (7-NI) for 24 hours (Fujibayashi et al., 2015). This concentration was confirmed in a dose-response experiment (FIG. 14A) using an MTT viability assay.

### MTT Cell Viability Assay:

Effects of 7-NI on neuroblastoma cell growth were determined using a colorimetric 3-(4,5-dimethylthiazol-2-yl)-2,5-diphenyl-tetrazoleum (MTT) assay (Sigma-Aldrich). Five or six parallels of each treatment were performed in each experiment.

siRNA Transfection:

SH-SY5Y cells were plated in 6 well plates, as well as on 22 mm² coverslips at a density of 6.0×10⁴ cells/cm² and transfected with 20 nM nNOS siRNA (NOSIHSS1072, Invitrogen) for 24 hours, with the use of Lipofectamine RNAi MAX (56530, Invitrogen) according to the manufacturer's protocol. Then cells were washed with phosphate-buffered saline (PBS) and harvested for WB and fixed for immunofluorescence (Fujibayashi et al., 2015).

### Clonogenic Survival Assay:

Cells were seeded into 6-well plates at concentrations of 500/well or 1000/well in triplicates. After 24 hours, 7-NI was added (100 μM for 24 hours), or cells were transiently transfected with siRNA-nNOS, as described above, before changing to serum-containing medium. Cells were incubated for 14 days to form visible colonies. Colonies were stained with crystal violet, washed with PBS, air-dried, and imaged. The colonies were visualized using ImageJ software. Cell colonies of greater than 50 cells were identified and counted (Lopez-Rivera et al., 2014).

### Measurement of NADPH-Diaphorase Activity:

The NADPH-diaphorase activity assay was used to determine the NOS activity based on the NADPH-dependent reduction of nitro-blue tetrazolium salt (NBT) to visible formazan (Amaroli and Chessa, 2012; Seckler et al., 2020). SH-SY5Y cells grown in six-well culture plates were lysed in 50 mM Tris-HCl buffer (pH 7.4), containing 0.1% Triton X-100, 2 mM EDTA, and a protease inhibitor cocktail (Sigma, St. Louis, MO). 100 μl of the lysate were added to 250 μl of 50 mM Tris-HCl, pH 7.5, containing 5 mM EDTA, 0.5 mM NBT and 0.5 mM NADPH. After a 15 min incubation period at 37° C., the production of formazan was measured spectrophotometrically at 585 nm. The data were expressed as absorbance units and normalized for the protein content of each sample. Protein concentration was measured by the bicinchoninic acid (BCA) protein assay using BSA as a standard (Ortiz-Ortiz et al., 2009). Released NO was measured using a Griess Assay Kit (Beyotime, Shanghai, China) according to the manufacturer's instructions.

### Nitrite Assay:

Released NO was measured with Griess assay Kit (Sigma-Aldrich, Cat. No. 23479) using the manufacturer's instructions.

### Western Blots (WB):

Cells were lysed in ice-cold RIPA buffer containing 50 mM Tris-HCl, 1% Nonidet P-40, 0.25% sodium deoxycholate, 150 mM NaCl, protease-, and phosphatase inhibitor cocktails, pH 7.4. After sonication, the lysates were centrifuged at 17,000 g for 30 minutes at 4° C., and the supernatant was collected. The protein concentrations were determined with the BCA protein assay. Proteins were resolved by SDS-PAGE, transferred to a PVDF membrane, and probed with the following antibodies: anti-pMTOR (rabbit, diluted 1:1000), anti-mTOR (rabbit, diluted 1:1000), anti-TSC2 (rabbit, diluted 1:1000), anti-nNOS (rabbit, diled 1:1000), anti-pAKT (rabbit, diluted 1:1000), anti-AKT (rabbit, diluted 1:1000), anti-pRPS6 (rabbit, diluted 1:1000), anti-RPS6 (mouse, diluted 1:1000), anti-synaptophysin (mouse, diluted 1:1000), anti-β-actin (rabbit, diluted 1:1000), anti-3-Nitrotyrosine (rabbit, diluted 1:5000) from Abcam. All primary antibodies, except anti-3-Nitrosine, were purchased from Cell Signaling Technology. The blots were then visualized using an enhanced chemiluminescence (ECL) system (Choi et al., 2018).

### Confocal Microscopy:

Cells cultured on coverslips coated with Poly-D-Lysine were washed three times with PBS and fixed in 4% paraformaldehyde for 15-20 min at room temperature. The fixed cells were washed three times with PBS and incubated in the presence of permeabilization buffer (PBS containing 0.1% Triton X-100) for 5 min at room temperature. After washing three times with PBS, the cells were blocked with blocking buffer (PBS containing 1% BSA, 1% goat serum, and 0.1% tritonX-100, pH 7.4) for 30 min at room temperature and then incubated overnight at 4° C. with the following antibodies: anti-nNOS (rabbit, diluted 1:200), anti-NeuN (mouse, diluted 1:200), anti-synaptophysin (rabbit, diluted 1:200), anti-3-Nitrotyrosine (mouse, diluted 1:200) from Abcam. All primary antibodies, except anti-3-Nitrosine, were purchased from Cell Signaling Technology. Then, the samples were incubated with Alexa 488- or Alexa 594-conjugated secondary antibodies for 2 hours and then mounted with DAPI-containing mounting media. The mounted slides were observed under a confocal microscope (Choi et al., 2018).

### Statistical Analysis:

Each experiment was repeated at least three times, with a satisfactory correlation achieved between the results of individual experiments. The data shown are the average of 3-4 culture dishes for each group. In each experiment, differences between groups were assessed using the one-way ANOVA followed by Fisher's LSD multiple comparison post hoc test, with values of P<0.05 considered to indicate statistical significance. Data are expressed as the mean±SEM.

### Example 14

### 7-NI Affects Neuroblastoma Cells Viability

FIGS. 14A-E show an optimal concentration of 7-NI (FIG. 14A), and successful knockdown of nNOS by siRNA (FIGS. 14B-E). FIG. 14A presents comparative plots showing the cell viability of SH-SY5Y as measured by MTT (the dose-response curve). The concentration of 7-NI for the cell treatment was 100 μM. Data were presented in percentage of 100. FIG. 14B is a representative blot of nNOS. 1, SH-SY5Y; 2, SH-SY5Y+negative control (NC); and 3, SH-SY5Y+si-nNOS. FIG. 14C presents representative fluorescence images of nNOS immunofluorescence. Blue color represents DAPI (a marker of nucleus), green color shows NeuN (a marker of neuron) and red color represents nNOS. FIG. 14D is a bar graph showing the relative abundance of nNOS. FIG. 14E is a bar graph showing the relative fluorescence intensity of nNOS.

### Example 15

### Inhibition of NO Production (Diaphorase Activity) in SH-SY5Y by 7-NI and Silencing of nNOS

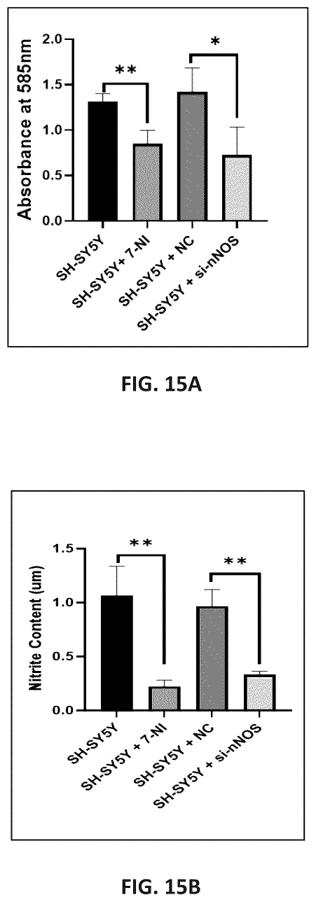

SH-SY5Y cells displayed high levels of NADPH diaphorase activity, which was significantly inhibited by the treatment with 7-NI or nNOS knockdown by siRNA (FIG. 15A). The Greiss assay also showed a reduction of nitrite content in SH-SY5Y cells treated with 7-NI or following the nNOS silencing (FIG. 15B).

### Example 16

### nNOS Inhibition/Knockdown Prevents NO-Mediated Tumor Proliferation

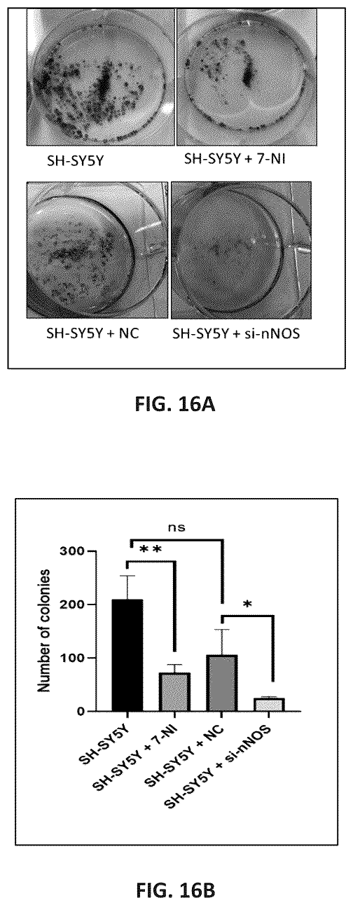

The clonogenic survival assay showed a decrease in the number of colonies following the nNOS silencing and 7-NI treatment of SH-SY5Y cells (FIGS. 16A-B). This points to the reduced proliferative capacity of the treated cells.

### Example 17

### nNOS Inhibition/Knockdown Prevents TSC-2 S-Nitrosylation and Reduces the Activity of the mTOR Signaling Pathway

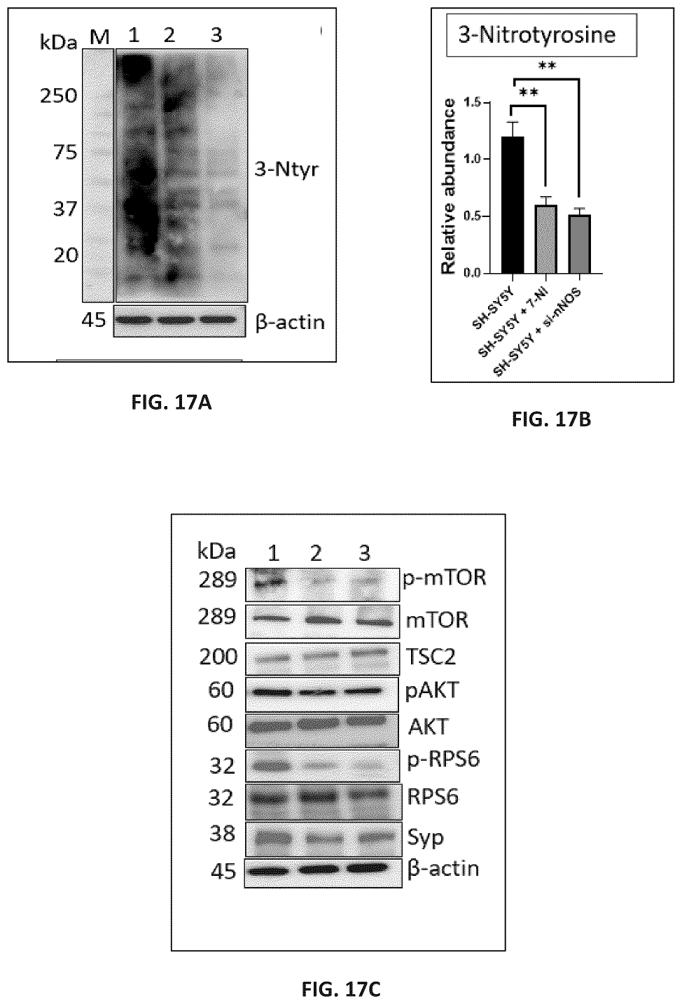

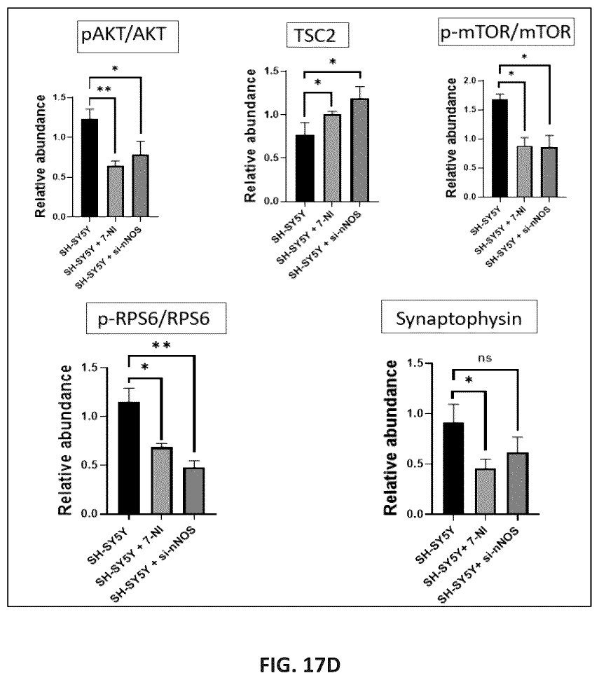

Treatment of SH-SY5Y cells with 7-NI halved 3-Ntyr levels. Similar changes in 3-Ntyr levels were observed in the cells after nNOS knockdown (FIGS. 17A-B). Treatment of SH-SY5Y cells with 7-NI or silencing of nNOS led to a significant increase in the levels of TSC-2, a key negative regulator of mTORCl signaling pathway (Way et al., 2009). These interventions also resulted in a decrease in phosphorylation of mTOR. Phosphorylation of an upstream component of this signaling pathway, protein kinase-B (AKT), and its downstream target, RPS6 (FIGS. 17C-D) was also reduced. 7-NI and the genetic ablation of nNOS significantly decreased the levels of synaptophysin (Syp, FIGS. 17C-D).

### Example 18

### Reduction in the Immunofluorescence of Synaptophysin after nNOS Knockdown or nNOS Inhibition

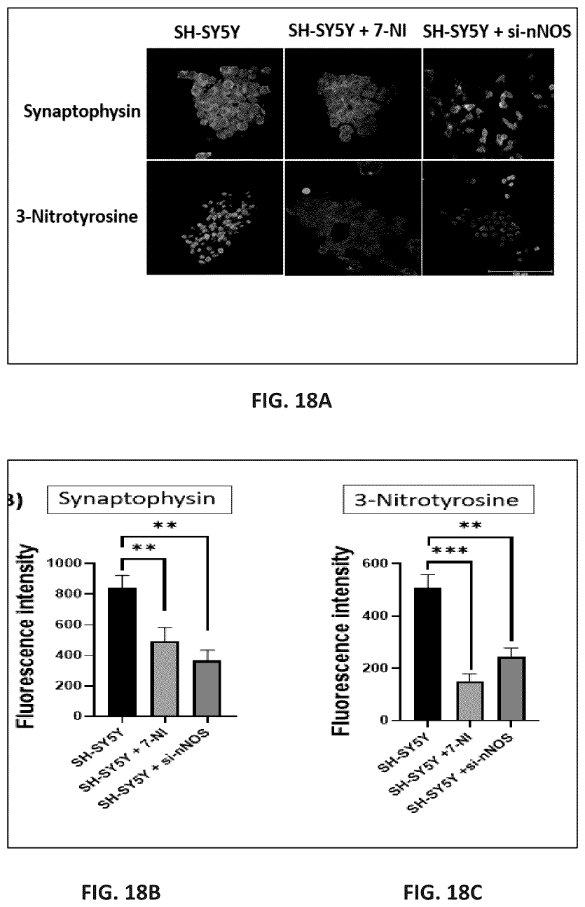

SH-SY5Y cells showed a relatively high intensity of 3-Ntyr and Syp immunofluorescence. Pharmacological inhibition or knockdown of nNOS significantly decreased the intensity of 3-Ntyr and Syp (FIGS. 18A-C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES CITED BY NUMERALS (Other References are Listed in the Document)

1) Guang S, Pang N, Deng X, Yang L, He F, Wu L, Chen C, Yin F and Peng J. Synaptopathology involved in autism spectrum disorder. Front Cell Neurosci. 2018; 12:470.
2) Stessman H A, Xiong B, Coe B P, Wang T, Hoekzema K, Fenckova M, Kvarnung M, Gerdts J, Trinh S and Cosemans N. Targeted sequencing identifies 91 neurodevelopmental-disorder risk genes with autism and developmental-disability biases. Nat Genet. 2017; 49:515-526.
3) Peça, J., et al., SHANK3 mutant mice display autistic-like behaviours and striatal dysfunction. Nature, 2011. 472 (7344): p. 437-442.
4) Feliciano P. Cntnap2−/− autism model. Nat Genet. 2011; 43:1053-1053.
5) Amal. H., et al., SHANK3 mutation in a mouse model of autism leads to changes in the S-nitroso-proteome and affects key proteins involved in vesicle release and synaptic function. Mol Psychiatry, 2020. 25 (8): p. 1835-1848.
6) Front. Neurosci., 31 Aug. 2020 |www(dot)doi(dot)org/10.3389/fnins.2020.00886 Neuronal Nitric Oxide Synthase Knockdown Within Basolateral Amygdala Induces Autistic-Related Phenotypes and Decreases Excitatory Synaptic Transmission in Mice, Xiaona Wang1 et al
7) I. A. Shkrob, T. W. Marin, The AHA Moment: Assessment of the Redox Stability of Ionic Liquids Based on Aromatic Heterocyclic Anions (AHAs) for Nuclear Separations and Electric Energy Storage, J Phys Chem B, 119 (2015) 14766-14779.
8) M. Kazhemekaite, A. Yuodvirshis, A. Vektarene, Preparation of the pure sodium salt of 1H-1,2,4-triazole, Chemistry of Heterocyclic Compounds, 34 (1998) 252-253.
9) Z. Wu, D. Li, G. Yan, H. Wang, S. Liu, J. Yang, G. Zhang, Heat-resistant and shape-memory metallo-supramolecules with simultaneously switchable fluorescence behavior supported by tridentate N3 group, Polymer, 208 (2020).
10) A. Cingolani, S. Galli, N. Masciocchi, L. Pandolfo, C. Pettinari, A. Sironi, The competition between acetate and pyrazolate in the formation of polynuclear Zn(II) coordination complexes, Dalton Trans, (2006) 2479-2486.
11) N. Masciocchi, G. A. Ardizzoia, S. Brenna, G. LaMonica, A. Maspero, S. Galli, A. Sironi, One-dimensional polymers containing strictly collinear metal ions: synthesis and XRPD characterization of homoleptic binary metal pyrazolates, Inorg Chem, 41 (2002) 6080-6089.
12) Z. Ullah, M. A. Bustam, Z. Man, N. Muhammad, A. S. Khan, Synthesis, characterization and the effect of temperature on different physicochemical properties of protic ionic liquids, RSC Advances, 5 (2015) 71449-71461.
13) G. Chen, S. Kang, Q. Ma, W. Chen, Y. Tang, Chemical and thermal stability of N-heterocyclic ionic liquids in catalytic C—H activation reactions, Magn Reson Chem, 52 (2014) 673-679.
14) C. Chopard, C. Lenoir, S. Rizzato, M. Vidal, J. Arpalahti, L. Gabison, A. Albinati, C. Garbay, J. Kozelka, Synthetic route to dinuclear platinum(II) complexes [{trans-PtCl(NH3)2}2(mu-L)]2+ (L=aliphatic or heterocyclic diamine) as potential antitumor agents, exploiting the mutual activation of hydroxido ligands and ammonium groups, Inorg Chem, 47 (2008) 9701-9705.
15) C. J. Adams, M. A. Kurawa, A. G. Orpen, Coordination chemistry in the solid state: synthesis and interconversion of pyrazolium salts, pyrazole complexes, and pyrazolate MOFs, Dalton Trans, 39 (2010) 6974-6984.

What is claimed is:

1. A method for the treatment of neuroblastoma in a subject in need, comprising administering to the subject an effective amount of a pharmaceutical composition comprising an active agent which reduces or inhibits nNOS enzymatic activity.

2. The method of claim 1, wherein the active agent is selected from 7-nitro-indazole (7-NI), L-thiocitrulline, S-methyl-L-thiocitrulline, S-methyl-L-thiocitrulline, N⁵-(1-iminobutyl)-L-ornithine (ethyl-L-NIO), N⁵-(1-imino-3-butenyl)-L-ornithine (vinyl-L-NIO), 3-bromo-7-nitroindazole (7-NI-Br), and methylene blue.

3. The method of claim 1, wherein said agent is 7-nitro-indazole (7-NI)).


SEQUENCE LISTING (14 sequences)

SEQ ID NO: 1-2 - skipped (2 entries)

SEQ ID NO: 3 - RNA, 25 nt, synthetic construct
  source 1..25: mol_type = other RNA
  misc_feature 1..25: note = SHANK3 siRNA
cgatgataagcagtttgcaaagctt

SEQ ID NO: 4 - RNA, 25 nt, synthetic construct
  source 1..25: mol_type = other RNA
  misc_RNA 1..25: note = nNOS SiRNA
ccttgtccaacatgctcctagagat

SEQ ID NO: 5 - RNA, 25 nt, synthetic construct
  source 1..25: mol_type = other RNA
  misc_feature 1..25: note = nNOS SiRNA
atctctaggagcatgttggacacgg

SEQ ID NO: 6 - RNA, 25 nt, synthetic construct
  source 1..25: mol_type = other RNA
  misc_feature 1..25: note = nNOS SiRNA
ccaatgtcatttctgttcgtctctt

SEQ ID NO: 7 - RNA, 25 nt, synthetic construct
  source 1..25: mol_type = other RNA
  misc_feature 1..25: note = nNOS SiRNA
aagagacgaacagaaatgacattgg

SEQ ID NO: 8 - RNA, 25 nt, synthetic construct
  source 1..25: mol_type = other RNA
  misc_feature 1..25: note = nNOS SiRNA
ccggagacatcattcttgcggtcaa

SEQ ID NO: 9 - RNA, 70 nt, synthetic construct
  source 1..70: mol_type = other RNA
  misc_feature 1..70: note = shRNA against nNOS
gtacctccccacatttatggcttgtttaagtcaagagcttaaacaagccataaatgtgggtttttggaaa

SEQ ID NO: 10 - RNA, 70 nt, synthetic construct
  source 1..70: mol_type = other RNA
  misc_feature 1..70: note = shRNA against nNOS
agcttttccaaaaacccacatttatggcttgtttaagctcttgacttaaacaagccataaatgtggggag

SEQ ID NO: 11 - RNA, 60 nt, synthetic construct
  source 1..60: mol_type = other RNA
  misc_feature 1..60: note = shRNA against nNOS
acctctcccaaagtgctgagattacaggtcaagagcctgtaatctcagcactttgggatt

SEQ ID NO: 12 - RNA, 60 nt, synthetic construct
  source 1..60: mol_type = other RNA
  misc_feature 1..60: note = shRNA against nNOS
caaaaatcccaaagtgctgagattacaggctcttgacctgtaatctcagcactttgggag

SEQ ID NO: 13 - RNA, 70 nt, synthetic construct
  source 1..70: mol_type = other RNA
  misc_feature 1..70: note = shRNA against nNOS
gtacctctccagttgtaacccacatttatgtcaagagcataaatgtgggttacaactggatttttggaaa

SEQ ID NO: 14 - RNA, 70 nt, synthetic construct
  source 1..70: mol_type = other RNA
  misc_feature 1..70: note = shRNA against nNOS
agcttttccaaaaatccagttgtaacccacatttatgctcttgacataaatgtgggttacaactggagag